(19) United States
(12) Patent Application Publication
(10) Pub. No.: US 2020/0138804 A1
(43) Pub. Date: May 7, 2020

(54) METHODS FOR TREATING CANCER

(71) Applicant: X4 Pharmaceuticals, Inc., Cambridge, MA (US)

(72) Inventors: Sudha Parasuraman, Cambridge, MA (US); Yan Wang, Cambridge, MA (US)

(21) Appl. No.: 16/625,006

(22) PCT Filed: Jun. 21, 2018
(86) PCT No.: PCT/US2018/038776

§ 371 Date: Dec. 20, 2019

Related U.S. Application Data
(60) Provisional application No. 62/523,091, filed on Jun. 21, 2017.

(51) Int. Cl. A61K 31/4709; A61K 38/05; A61K 39/395; A61K 45/06; A61K 33/243; A61K 31/337; A61K 31/4245; A61K 31/405; A61K 31/4188; A61K 31/53; A61K 31/4025; A61K 31/47; A61K 31/502; A61K 31/55; A61K 31/4462; A61K 31/167; A61K 38/15; A61K 31/404; A61K 31/18; A61K 31/4406; A61K 31/5025; A61K 31/4439; A61K 31/553; A61K 31/519; A61K 31/52; A61K 31/506; A61P 35/00

(52) U.S. Cl. CPC A61K 31/4709; A61P 35/00; A61K 39/3955; A61K 45/06; A61K 33/243; A61K 31/337; A61K 31/4245; A61K 31/405; A61K 31/4188; A61K 31/53; A61K 31/4025; A61K 31/47; A61K 31/502; A61K 31/55; A61K 31/4462; A61K 31/167; A61K 38/15; A61K 31/404; A61K 31/18; A61K 31/4406; A61K 31/5025; A61K 31/4439; A61K 31/553; A61K 31/519; A61K 31/52; A61K 31/506; A61K 38/05

(57) ABSTRACT

The present invention relates to methods of treating cancer, in which a CXCR4 inhibitor such as X4P-001 or a pharmaceutically acceptable salt thereof or pharmaceutical composition thereof is administered in combination with an additional therapeutic agent, such as an immune checkpoint inhibitor. The methods demonstrate surprising results, including regression of disease, with comparatively little toxicity.

### CROSS-REFERENCE TO RELATED APPLICATIONS

This application claims the benefit of priority to United States Provisional Patent Application serial number U.S. 62/523,091, filed Jun. 21, 2017, the entirety of which is hereby incorporated by reference.

### FIELD OF THE INVENTION

The present invention relates to methods for treating cancer, for example, methods for treatment of patients with a cancer such as a solid tumor.

### BACKGROUND OF THE INVENTION

Cancer represents a continuing and significant threat to global human health. It is increasingly clear that cancer is a collection of heterogeneous and multifaceted diseases with less in common than previously thought. The genetic and phenotypical heterogeneity of cancer represents both a challenge and an opportunity. The challenge is that no single approach to treating all cancers appears imminent. However, the opportunity is that multiple means of treating specific types of cancers continue to present themselves with each discovery of a new mechanism for tumorigenesis, angiogenesis, metastasis, and other processes on which cancers depend. Harnessing new mechanisms for treating cancers that depend on these mechanisms represents a promising means of delivering therapeutics that meet the ongoing and urgent need for effective cancer therapeutics.

Chemokines influence a number of physiological and pathological processes, especially a group of such processes relating to cell homing and migration. The chemokine CXCL12 (also known as stromal cell-derived factor-1) binds CXCR4 (C-X-C receptor type 4), a G-protein-coupled receptor that increases intracellular calcium and influences processes such as cell adhesion, chemotaxis, survival, proliferation, and gene transcription by various divergent pathways. CXCR4 was initially discovered for its involvement in HIV entry and leukocyte trafficking. It is also overexpressed in more than 23 human cancers. For example, CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

These data underscore the significant, unmet need for study of CXCR4 inhibitors to treat cellular proliferative disorders that result from overexpression or aberrant expression of CXCR4.

### SUMMARY OF THE INVENTION

It has now been found that CXCR4 inhibitors such as X4P-001 are useful in treating a variety of cellular proliferative disorders, such as those described herein.

CXCR4 inhibitors such as the compound X4P-001, or a pharmaceutically acceptable salt thereof or pharmaceutical composition thereof, as described in greater detail below, are useful both as a monotherapy and as a combination therapy with one or more other therapeutic agents described herein. Accordingly, in one aspect, the present invention provides a method of treating a cancer, such as those described herein, by administering to a patient in need thereof an effective amount of a CXCR4 inhibitor such as X4P-001, or a pharmaceutically acceptable salt thereof or pharmaceutical composition thereof. In some embodiments, the method further includes co-administering simultaneously or sequentially an effective amount of one or more additional therapeutic agents, such as those described herein.

### BRIEF DESCRIPTION OF THE DRAWINGS

FIG. 1 shows the results of combination therapy of X4P-001 with anti-murine PD-1 (RMP1-14) in a syngeneic mouse tumor model (MC38). The results of this experiment for Groups 1 through 4 demonstrate enhanced activity for the combination therapy due to increased mouse survival.

FIG. 2 shows tumor volume in mice treated with control; X4P-001 alone; anti-PD-1 (nivolumab) alone; or X4P-001 in combination with anti-PD-1.

FIG. 3 shows the immunohistochemical staining of biopsies from a melanoma patient prior to treatment (Day 1) and after three (3) weeks (i.e., at Week 4) of treatment with the CXCR4 small molecule inhibitor X4P-001. CD8+ T-cells are stained red, and are indicated by green arrows. At Week 4, marked increases in CD8+ T-cells are observed especially in the central tumor margin.

### DETAILED DESCRIPTION OF CERTAIN EMBODIMENTS OF THE INVENTION

Cancer immunotherapy and targeted therapies, such as with ipilimumab or a PD-1 antagonist or antibody, can produce long-lasting responses against metastatic cancer having a wide range of histologies. However, an improved understanding of how some tumors avoid the immune response is required in order to broaden their applicability. It is difficult to study such mechanisms because the interactions between the immune system and cancer cells are continuous and dynamic, meaning that they evolve over time from the initial establishment of the cancer through development of metastasis, which allows the tumor to avoid the immune system. It is now understood that the use of immunotherapy alone may be hindered or rendered ineffective by primary, adaptive, or acquired resistance mechanisms (“immune escape”). See, e.g., Sharma, P. et al., Cell 2017, 168, 707-723.

There is also some evidence suggesting that the CXCL12/CXCR4 axis may be a cause of angiogenic escape, which is the loss or lack of tumor responsiveness to angiogenesis inhibitors. In animal cancer models, interference with CXCR4 function has been demonstrated to alter the TME and sensitize the tumor to immune attack by multiple mechanisms such as elimination of tumor re-vascularization and increasing the ratio of CD8+ T cells to Treg cells. These effects result in significantly decreased tumor burden and increased overall survival in xenograft, syngeneic, and transgenic cancer models. See, e.g., Vanharanta et al. (2013) Nat Med 19: 50-56; Gale and McColl (1999) BioEssays 21: 17-28; Highfill et al. (2014) Sci Transl Med 6: ra67; Facciabene et al. (2011) Nature 475: 226-230.

Recent studies demonstrate that CXCR4/CXCL12 is a primary receptor-ligand pair that cancer cells and surrounding stromal cells use to block normal immune function and promote angiogenesis through the trafficking of T-effector and T-regulatory cells, as well as myeloid derived suppressor cells (MDSCs), in the tumor microenvironment. Cancer cell CXCR4 overexpression contributes to tumor growth, invasion, angiogenesis, metastasis, relapse, and therapeutic resistance. Accordingly, CXCR4 antagonism represents a means to disrupt tumor-stromal interactions, sensitize cancer cells to cytotoxic drugs, and/or reduce tumor growth and metastatic burden.

X4P-001 is an orally bioavailable, small molecule inhibitor of CXCR4. It has now been found that CXCR4 inhibitors such as X4P-001, or a pharmaceutically acceptable salt thereof or pharmaceutical composition thereof, as described in greater detail below, is useful both as a monotherapy and as a combination therapy with one or more other therapeutic agents described herein. Accordingly, in one aspect, the present invention provides a method of treating a cancer, such as those described herein, by administering to a patient in need thereof an effective amount of X4P-001, or a pharmaceutically acceptable salt thereof or pharmaceutical composition thereof. In some embodiments, the method includes co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of X4P-001 and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory.

The benefit of neoadjuvant chemo- and immunotherapy has been demonstrated in several operable cancers. Compared to adjuvant therapy, neoadjuvant therapy in patients with locally and regionally advanced cancer has several potential benefits, such as (1) reducing the size of the primary and metastatic tumor increases the probability of achieving negative margin resection; (2) tumor exposure to potentially effective systemic therapy is increased while blood and lymphatic vessels remain intact; and (3) collection of pre- and intra-operative samples of tumor tissue following neoadjuvant therapy offers real-time, in vivo assessment of the effects of the therapy on the tumor cells, the tumor microenvironment (TME), and the immune system.

In some embodiments, by attacking multiple aspects of the TME, the effectiveness of conventional anticancer or antitumor therapies is augmented. Accordingly, in some embodiments the present invention provides combinations of therapeutics, for example targeted therapeutics, such as kinase inhibitors, with immunomodulatory therapies, such as immune checkpoint inhibitors. In some embodiments, by adding the use of a CXCR4 inhibitor such as X4P-001, the problem of acquired resistance to targeted therapeutics and/or immunomodulatory therapies is overcome at least in part, or emergence of resistance is delayed, such that an improved clinical outcome may be obtained, for example in resistant, refractory, or previously-treated cancers. Additionally, in some embodiments, the inclusion of a CXCR4 inhibitor such as X4P-001 may sensitize the TME, such that lower doses of cytotoxic compounds or an additional cancer therapeutic may exhibit increased efficacy.

In some embodiments, a combination therapy of a CXCR4 inhibitor, such as X4P-001 or a pharmaceutically acceptable salt thereof, in combination with a chemotherapeutic, targeted therapeutic, or immunomodulatory therapy, increases the effectiveness of such therapies, and/or may increase the period of time that such therapies are effective before a patient's cancer becomes resistant or refractory to such treatment. By doing so, such therapies effect a full or partial response or remission and/or delay the time of progression of disease.

X4P-001, formerly designated AMD11070, is a potent, orally bioavailable CXCR4 antagonist (see Montane et al. (2011) J Clin Invest 121: 3024-8), that has demonstrated activity in solid and liquid tumor models (see Acharyya et al. (2012) Cell 150: 165-78, and unpublished data) and has previously (under the designations AMD070 and AMD11070) been in Phase 1 and 2a trials involving a total of 71 healthy volunteers (see Montane et al. (2011) J Clin Invest 121: 3024-8; Zhao et al. (2012) J Clin Invest 122: 4094-4104; Silva et al. (2008) Science 319: 617-20) and HIV-infected subjects (see Schlabach et al. (2008) Science 319: 620-24; Shen et al. (2013) Tumour Biol 34: 1839-45). These studies demonstrated that oral administration of up to 400 mg BID for 3.5 days (healthy volunteers) and 200 mg BID for 8-10 days (healthy volunteers and HIV patients) was well-tolerated with no pattern of adverse events or clinically significant laboratory changes. These studies also demonstrated pharmacodynamic activity, with dose- and concentration-related changes in circulating white blood cells (WBCs); and a high volume of distribution (VL), suggesting high tissue penetration.

Plerixafor (formerly designated AMD3100, now marketed as Mozobil) is the only CXCR4 antagonist currently FDA approved. Plerixafor is administered by subcutaneous injection and has a very short half-life; the only FDA-approved indication is for courses of 3 to 5 days to release HSC from the bone marrow into the peripheral blood for harvesting. Both X4P-001 and plerixafor have been studied in murine models of melanoma, renal cell carcinoma, and ovarian cancer and have demonstrated significant anti-tumor activity, including decreased metastasis and increased overall survival. The treatment effect has been associated with decreased presence of myeloid-derived suppressor cells (MDSCs) in the TME and increased presence of tumor-specific CD-8+ effector cells. See D'Alterio, et al. (2012) Cancer Immunol Immunother 61:1713-1720; Feig, et al. (2013) PNAS 110:20212-20217; and Zhang et al. (2006) Cancer Biol Ther. 5:1034-1312.

Without wishing to be bound by any particular theory, it is believed that administration of X4P-001 to a patient with cancer will increase the density of CD8+ T cells among the patient's tumor or cancer cells and that this effect will be sustained or increased when X4P-001 is given in combination with one or more additional anticancer agents or therapies such as a chemotherapeutic, targeted therapeutic or immunomodulatory therapy. Because X4P-001 is well-tolerated in the body and may increase the ability of the body to mount a robust anti-tumor immune response, in some embodiments administering X4P-001 in such a combination substantially increases the objective response rate in multiple tumor types, the frequency of durable long-term responses, and/or overall survival, without significantly increasing the adverse effects on patients receiving such therapies.

It is further anticipated that such results will be achieved with comparatively little toxicity because CXCR4-targeted drugs are not be expected to induce cell cycle arrest in bone marrow and other normal proliferating cell populations. Accordingly, the present invention provides significant advantages in treatment outcomes utilizing the low toxicity and robust effects of the CXCR4 inhibitor X4P-001 on MIDSC trafficking, differentiation and tumor cell gene expression in a cancer.

In some embodiments, administration of X4P-001 or a pharmaceutically acceptable salt thereof or pharmaceutical composition thereof increases the density of CD8+ T cells, thereby resulting in increased anti-tumor immune attack. In some embodiments, administration of X4P-001 or a pharmaceutically acceptable salt thereof or pharmaceutical composition thereof additionally decreases neoangiogenesis and tumor vascular supply. In some embodiments, administration of X4P-001 or a pharmaceutically acceptable salt thereof or pharmaceutical composition thereof interferes with the autocrine effect of increased expression by tumors of both CXCR4 and its only ligand, CXCL12, thereby reducing cancer cell metastasis.

It is further believed that CXCR4 inhibitors, such as X4P-001 or a pharmaceutically acceptable salt thereof or pharmaceutical composition thereof may be used in a synergistic combination with anti-angiogenic agents. In some embodiments, such combinations delay the emergence of resistance, sensitize tumors to immunomodulation, and hence synergize with immune modulating agents such as checkpoint inhibitors, and/or sensitize tumors to chemotherapeutic agents and radiation. Hence, X4P-001 or a pharmaceutically acceptable salt thereof or pharmaceutical composition thereof may be combined with standard and state-of-the-art treatments including chemotherapy and radiation treatments.

In some embodiments, X4P-001 or a pharmaceutically acceptable salt thereof or pharmaceutical composition thereof is used in combination with an approved cancer therapy such as radiation, a chemotherapeutic, or an immunotherapy or targeted therapeutic such as a tyrosine kinase inhibitor or checkpoint inhibitor.

In one aspect, the present invention provides a method of treating cancer in a patient in need thereof, wherein said method comprises administering to said patient X4P-001 or a pharmaceutically acceptable salt thereof in combination with one or more additional therapeutic agents, such as one or more immunostimulatory therapeutic compounds.

In some embodiments, the one or more immunostimulatory therapeutic compounds are selected from elotuzumab, mifamurtide, an agonist or activator of a toll-like receptor, or an activator of RORγt.

In some embodiments, the method further comprises administering to said patient a third therapeutic agent, such as an immune checkpoint inhibitor. In some embodiments, the method comprises administering to the patient in need thereof three therapeutic agents selected from X4P-001 or a pharmaceutically acceptable salt thereof, an immunostimulatory therapeutic compound, and an immune checkpoint inhibitor.

In some embodiments, the immune checkpoint inhibitor is selected from nivolumab, pembrolizumab, ipilimumab, avelumab, durvalumab, atezolizumab, or pidilizumab.

In another aspect, the present invention provides a method of treating cancer in a patient in need thereof, wherein said method comprises administering to said patient X4P-001 or a pharmaceutically acceptable salt thereof in combination with one or more additional therapeutic agents selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor.

In some embodiments, the IDO inhibitor is selected from epacadostat, indoximod, capmanitib, GDC-0919, PF-06840003, BMS:F001287, Phy906/KD108, or an enzyme that breaks down kynurenine.

In some embodiments, the PARP inhibitor is selected from olaparib, rucaparib, or niraparib.

In some embodiments, the HDAC inhibitor is selected from vorinostat, romidepsin, panobinostat, belinostat, entinostat, or chidamide.

In some embodiments, the CDK 4/6 inhibitor is selected from palbociclib, ribociclib, abemaciclib or trilaciclib.

In some embodiments, the method further comprises administering to said patient a third therapeutic agent, such as an immune checkpoint inhibitor. In some embodiments, the method comprises administering to the patient in need thereof three therapeutic agents selected from X4P-001 or a pharmaceutically acceptable salt thereof, a second therapeutic agent selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor, and a third therapeutic agent selected from an immune checkpoint inhibitor. In some embodiments, the immune checkpoint inhibitor is selected from nivolumab, pembrolizumab, ipilimumab, avelumab, durvalumab, atezolizumab, or pidilizumab.

In some embodiments, the PI3K inhibitor is selected from idelalisib, alpelisib, taselisib, pictilisib, copanlisib, duvelisib, PQR309, or TGR1202.

In another aspect, the present invention provides a method of treating cancer in a patient in need thereof, wherein said method comprises administering to said patient X4P-001 or a pharmaceutically acceptable salt thereof in combination with one or more additional therapeutic agents selected from a platinum-based therapeutic, a taxane, a nucleoside inhibitor, or a therapeutic agent that interferes with normal DNA synthesis, protein synthesis, cell replication, or will otherwise inhibit rapidly proliferating cells.

In some embodiments, the platinum-based therapeutic is selected from cisplatin, carboplatin, oxaliplatin, nedaplatin, picoplatin, or satraplatin.

In some embodiments, the taxane is selected from paclitaxel, docetaxel, albumin-bound paclitaxel, cabazitaxel, or SID530.

In some embodiments, the therapeutic agent that interferes with normal DNA synthesis, protein synthesis, cell replication, or will otherwise interfere with the replication of rapidly proliferating cells is selected from trabectedin, mechlorethamine, vincristine, temozolomide, cytarabine, lomustine, azacitidine, omacetaxine mepesuccinate, asparaginase Envinia chrysanthemi, eribulin mesylate, capacetrine, bendamustine, ixabepilone, nelarabine, clorafabine, trifluridine, or tipiracil.

In some embodiments, the method further comprises administering to said patient a third therapeutic agent, such as an immune checkpoint inhibitor. In some embodiments, the method comprises administering to the patient in need thereof three therapeutic agents selected from X4P-001 or a pharmaceutically acceptable salt thereof, a second therapeutic agent selected from a platinum-based therapeutic, a taxane, a nucleoside inhibitor, or a therapeutic agent that interferes with normal DNA synthesis, protein synthesis, cell replication, or will otherwise inhibit rapidly proliferating cells, and a third therapeutic agent selected from an immune checkpoint inhibitor.

In some embodiments, the immune checkpoint inhibitor is selected from nivolumab, pembrolizumab, ipilimumab, avelumab, durvalumab, atezolizumab, or pidilizumab.

In some embodiments, any one of the foregoing methods further comprises the step of obtaining a biological sample from the patient and measuring the amount of a disease-related biomarker.

In some embodiments, the biological sample is a blood sample.

In some embodiments, the disease-related biomarker is selected from circulating CD8+ T cells or the ratio of CD8+ T cells:Treg cells.

In some embodiments, the cancer is selected from hepatocellular carcinoma, ovarian cancer, ovarian epithelial cancer, fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering X4P-001, or a pharmaceutically acceptable salt thereof, to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the present invention provides a method for treating a cancer selected from leukemia or a cancer of the blood, comprising administering to a patient in need thereof an effective amount of X4P-001 or a pharmaceutically acceptable salt thereof or pharmaceutical composition thereof, optionally in combination with an additional therapeutic agent such as those described herein. In some embodiments, the cancer is selected from acute myeloid leukemia (AML), chronic myeloid leukemia (CML), acute lymphocytic leukemia (ALL), chronic lymphocytic leukemia (CLL), or a virally induced leukemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the cancer is an advanced cancer, such as an advanced kidney cancer or advanced renal cell carcinoma.

In some embodiments, the present invention provides a method for treating refractory cancer in a patient in need thereof comprising administering to a patient in need thereof an effective amount of X4P-001 or a pharmaceutically acceptable salt thereof or pharmaceutical composition thereof, optionally in combination with an additional therapeutic agent such as those described herein.

In certain embodiments, the patient was previously administered a protein kinase inhibitor. In some embodiments, the patient was previously administered a VEGF-R antagonist. In certain embodiments, the patient was previously administered an immune checkpoint inhibitor. In some embodiments, the patient was previously administered an immune checkpoint inhibitor selected from nivolumab (Opdivo®, Bristol-Myers Squibb), pembrolizumab (Keytruda®, Merck), or ipilumumab (Yervoy®, Bristol-Myers Squibb).

In some embodiments, X4P-001, or a pharmaceutically acceptable salt thereof or pharmaceutical composition thereof, is administered to a patient in a fasted state.

### Co-Administered Therapeutic Agents

In certain embodiments, X4P-001 or a pharmaceutically acceptable salt thereof, or another CXCR4 antagonist, is administered in combination with an additional therapeutic agent. In some embodiments, X4P-001 or a pharmaceutically acceptable salt thereof, or another CXCR4 antagonist, is administered in combination with one additional therapeutic agent. In some embodiments, X4P-001 or a pharmaceutically acceptable salt thereof, or another CXCR4 antagonist, is administered in combination with two additional therapeutic agents. In some embodiments, X4P-001 or a pharmaceutically acceptable salt thereof, or another CXCR4 antagonist, is administered in combination with three or more additional therapeutic agents. In some embodiments, one of the additional therapeutic agents is an immune checkpoint inhibitor.

Research into the mechanisms of acquired resistance to VEGF-targeted therapies has demonstrated that treatment with sunitinib treatment resulted in a marked increase in the infiltration of renal cell carcinoma (RCC) xenografts with CD11b+/Gr-1+ myeloid-derived suppressor cells (MDSC) (1). These cells have been repeatedly implicated in the development of resistance to a diverse array of anticancer therapies, including VEGF-targeted agents (2-5). Coadministration of a CXCR4 inhibitor such as X4P-001 or a pharmaceutically acceptable salt thereof would decrease tumor resistance to VEGF-targeted agents. Accordingly, in some embodiments, the present invention provides a method of treating a cancer, such as those described herein, by administering to a patient in need thereof an effective amount of X4P-001, or a pharmaceutically acceptable salt thereof or pharmaceutical composition thereof, in combination with an additional therapeutic agent selected from a VEGF inhibitor. In some embodiments, the VEGF inhibitor is one of those described herein, such as sunitinib or axitinib.

In one aspect, the present invention provides a method of treating an advanced cancer, comprising administering a CXCR4 inhibitor, such as X4P-001 or a pharmaceutically acceptable salt thereof or pharmaceutical composition thereof, either as a single agent (monotherapy), or in combination with a chemotherapeutic, a targeted therapeutic, such as a kinase inhibitor, and/or an immunomodulatory therapy, such as an immune checkpoint inhibitor. In some embodiments, the immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the additional therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, the additional therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In some embodiments, the additional therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In some embodiments, the additional therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, the additional therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In some embodiments, the additional therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In some embodiments, the additional therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In some embodiments, the additional therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In some embodiments, the additional therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In some embodiments, the additional therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In some embodiments, the additional therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, the additional therapeutic agent is a folic acid inhibitor. Approved folic acid inhibitors useful in the present invention include pemetrexed (Alimta®, Eli Lilly).

In some embodiments, the additional therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, the additional therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, the additional therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, the additional therapeutic agent is a glutaminase inhibitor. Glutaminase inhibitors being studied which may be used in the present invention include CB-839 (Calithera Biosciences).

In some embodiments, the additional therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, the additional therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, the additional therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Envinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cab azitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, the additional therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In some embodiments, the additional therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In some embodiments, the additional therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, the present invention provides a method of treating prostate cancer comprising administering to a patient in need thereof an effective amount of a CXCR4 antagonist such as X4P-001 or a pharmaceutically acceptable salt thereof or pharmaceutical composition thereof in combination with an additional therapeutic agent that interferes with the synthesis or activity of androgens. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, the additional therapeutic agent is a selective estrogen receptor modulator (SERM), which interferes with the synthesis or activity of estrogens. Approved SERMs useful in the present invention include raloxifene (Evista®, Eli Lilly).

In some embodiments, the additional therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, the additional therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, the additional therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFβ “trap.”

### Co-Administered Therapeutic Agents—Targeted Therapeutics and Immunomodulatory Drugs

In some embodiments, the additional therapeutic agent co-administered with X4P-001 or a pharmaceutically acceptable salt thereof or pharmaceutical composition thereof is selected from a targeted therapeutic or immunomodulatory drug. Adjuvant therapies with targeted therapeutics or immunomodulatory drugs have shown promising effectiveness when administered alone but are limited by the development of tumor immunity over time or evasion of the immune response.

In some embodiments, the present invention provides a method of treating cancer, such as a cancer described herein, comprising administering to a patient in need thereof an effective amount of a CXCR4 antagonist such as X4P-001 or a pharmaceutically acceptable salt thereof or pharmaceutical composition thereof in combination with an additional therapeutic agent such as a targeted therapeutic or an immunomodulatory drug. In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In other embodiments, the immunomodulatory therapeutic is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase-(TK−) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, the additional therapeutic agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, the present invention comprises administering to said patient a CXCR4 antagonist such as X4P-001 or a pharmaceutically acceptable salt thereof in combination with a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cellsengineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

### Co-Administered Therapeutic Agents—Immunostimulatory Drugs

In some embodiments, the additional therapeutic agent co-administered with X4P-001 or a pharmaceutically acceptable salt thereof or pharmaceutical composition thereof is an immunostimulatory drug. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the present invention provides a method of treating cancer, such as a cancer described herein, comprising administering to a patient in need thereof an effective amount of a CXCR4 antagonist such as X4P-001 or a pharmaceutically acceptable salt thereof or pharmaceutical composition thereof in combination with an additional therapeutic agent such as a immunostimulatory drug, such as an immune checkpoint inhibitor. In some embodiments, the X4P-001 and the checkpoint inhibitor are administered simultaneously or sequentially. In some embodiments, X4P-001 or a pharmaceutically acceptable salt thereof is administered prior to the initial dosing with the immune checkpoint inhibitor. In certain embodiments, the immune checkpoint inhibitor is administered prior to the initial dosing with X4P-001 or a pharmaceutically acceptable salt thereof.

In certain embodiments, the immune checkpoint inhibitor is selected from a PD-1 antagonist, a PD-L1 antagonist, or a CTLA-4 antagonist. In some embodiments, a CXCR4 antagonist such as X4P-001 or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech).

Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Nivolumab (Opdivo®, BMS-93568/MDX1106; Bristol-Myers Squibb), is a fully human IgG4 monoclonal antibody that acts as an immunomodulator by binding to the programmed cell death 1 (PD-1) receptor and selectively blocking interaction with its ligands PD-L1 and PD-L2. The structure and other properties of nivolumab are specified at http://www.drugbank.ca/drugs/DB09035, accessed on Mar. 14, 2016, the disclosure of which is hereby incorporated herein. Nivolumab is approved for use in treatment of patients with advanced renal cell carcinoma who have received prior anti-angiogenic therapy; as a single agent in certain types of unresectable or metastatic melanoma; in treating unresectable or metastatic melanoma or in combination with ipilimumab in treating unresectable or metastatic melanoma; and for treatment of metastatic non-small cell lung cancer and progression on or after platinum-based chemotherapy. Additionally, nivolumab has been tested or mentioned as a possible treatment in other oncologic indications, including solid tumors; skin melanoma; glioblastoma; glioma; gliosarcoma; astrocytoma; brain cancer; leukemia; acute myeloid leukemia; chronic myeloid leukemia; chronic lymphocytic leukemia; advanced liver cancer or hepatocellular carcinoma; uveal melanoma; prostate cancer; pancreatic neoplasm and pancreatic cancer; bladder cancer; colorectal cancer; myelodysplastic syndrome; Hodgkin Lymphoma; Non-Hodgkin Lymphoma; multiple myeloma; cervical cancer; endometrial cancer; uterine cancer; ovarian cancer and ovarian carcinoma; peritoneal carcinoma; head and neck squamous cell cancer; gastric cancer; esophageal cancer; Kaposi sarcoma; breast neoplasm, breast adenocarcinoma and breast cancer; bone sarcoma; soft tissue sarcoma; meningiomas; and mesothelioma.

In a phase 3 trial of over 800 patients with advanced clear-cell renal cell carcinoma, for which they had received previous treatment with one or two regimens of antiangiogenic therapy, were randomly assigned to receive 3 mg/kg body weight of nivolumab, intravenously every two weeks, or a 10 mg everolimus tablet orally daily. Patients treated with nivolumab exhibited longer median overall survival, decreased hazard ratio for death, and higher objective response rate than those patients treated with nivolumab (25%) compared to everolimus (5%) (P<0.001), with lower incidence of Grade 3 or 4 treatment-related adverse events (Motzer et al. (2015), New England Journal of Medicine, 373:1803-1813). Accordingly, in some embodiments, the present invention provides a method of treating advanced clear-cell renal cell carcinoma, comprising administering to a patient in need thereof an effective amount of a CXCR4 antagonist such as X4P-001 or a pharmaceutically acceptable salt thereof or pharmaceutical composition thereof in combination with nivolumab or everolimus, optionally wherein that patient has received previous treatment with a regimen of antiangiogenic therapy.

Generally, the amount of nivolumab or other immune checkpoint inhibitor useful in the present invention will be dependent upon the size, weight, age and condition of the patient being treated, the severity of the disorder or condition, and the discretion of the prescribing physician. For example, in its current prescribed labeling for unresectable or metastatic renal cell carcinoma, the recommended course of administration for nivolumab is 3 mg/kg as an intravenous infusion over 60 minutes every two weeks, until disease progression or unacceptable toxicity. In the discretion of the clinician, depending upon individual tolerance, the prescribed dose of nivolumab may be increased, for example, increased in dosage and/or frequency. In the discretion of the clinician, together with the warnings provided with prescribing information, administration of nivolumab may be discontinued, or the dose reduced in the case of significant adverse effects. In some embodiments, nivolumab is administered in the methods of the present invention according to the labeling guidelines above.

In some embodiments, the present invention provides a method for treating a patient by administering a CXCR4 antagonist such as X4P-001 or a pharmaceutically acceptable salt thereof in combination with an immunostimulatory therapeutics. Approved immunostimulatory therapeutics which may be used in the present invention include elotuzumab (anti-SLAMF7-antibody, Empliciti®, Bristol-Myers Squibb). Immunostimulatory compounds being studied that may be used in the present invention include mifamurtide (Mepact®, Takeda Oncology).

Another immunostimulatory therapeutic that may be used in the present invention is recombinant human interleukin 15 (rhIL-15). rhIL-15 has been tested in the clinic as a therapy for melanoma and renal cell carcinoma (NCT01021059 and NCT01369888) and leukemias (NCT02689453). Another immunostimulatory therapeutic that may be used in the present invention is recombinant human interleukin 12 (rhIL-12). Another suitable IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). Recombinant human interleukin 12 (rhIL-12) has been tested in the clinic for many oncological indications, for example, as a therapy for lymphoma (NM-IL-12, Neumedicines, Inc.), (NCT02544724 and NCT02542124).

Another paradigm for immune-stimulation is the use of oncolytic viruses. In some embodiments, the present invention provides a method for treating a patient by administering a CXCR4 antagonist such as X4P-001 or a pharmaceutically acceptable salt thereof or pharmaceutical composition thereof in combination with an immunostimulatory therapy such as oncolytic viruses. Approved immunostimulatory oncolytic viruses which may be used in the present invention include talimogene laherparepvec (live, attenuated herpes simplex virus, Imlygic®, Amgen).

In some embodiments, the additional therapeutic agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. An activator of RORγt, that is being studied which may be used in the present invention is LYC-55716 (Lycera), which is currently being evaluated in clinical trials for the treatment of solid tumors (NCT02929862).

In some embodiments, the additional therapeutic agent is an agonist or activator of toll-like receptors (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

In some embodiments, the additional therapeutic agent is an immune checkpoint inhibitor. In some embodiments, the carcinoma is resectable and metastatic. In other embodiments, the carcinoma is unresectable and metastatic. In some embodiments, the immune checkpoint inhibitor is nivolumab.

In some embodiments, the present invention provides a method for treating a refractory cancer in a patient, wherein said method comprises administering to said patient an effective amount of a CXCR4 antagonist such as X4P-001 or a pharmaceutically acceptable salt thereof or pharmaceutical composition thereof in combination with an immune checkpoint inhibitor. In some embodiments, the refractory cancer is metastatic renal cell carcinoma whose tumors express PD-L1, and who have disease progression after treatment with anti-angiogenic therapy or platinum-containing chemotherapy. In some embodiments, the refractory cancer is metastatic renal cell carcinoma and the immune checkpoint inhibitor is nivolumab.

In some embodiments of the disclosed methods, X4P-001, or a pharmaceutically acceptable salt thereof, is administered to a patient in need thereof in a fasted state and the immune checkpoint inhibitor is administered to the patient in either a fasted or fed state.

In certain embodiments, the present invention provides a method for treating cancer in a patient, wherein said method comprises administering to said patient an effective amount of a CXCR4 antagonist such as X4P-001 or a pharmaceutically acceptable salt thereof or pharmaceutically composition thereof in combination with an immune checkpoint inhibitor, further comprising the step of obtaining a biological sample from the patient and measuring the amount of a disease-related biomarker. In some embodiments, the biological sample is a blood sample. In certain embodiments, the disease-related biomarker is circulating CD8+ cells, plasma levels of PD-1, and/or plasma levels of PDL-1.

In certain embodiments, the present invention provides a method for treating advanced cancer, such as metastatic renal cell carcinoma, in a patient in need thereof, wherein said method comprises administering to said patient an effective amount of X4P-001 or a pharmaceutically acceptable salt thereof or pharmaceutical composition thereof in combination with nivolumab, further comprising the step of obtaining a biological sample from the patient and measuring the amount of a disease-related biomarker. In some embodiments, the biological sample is a blood sample. In certain embodiments, the disease-related biomarker is circulating CD8+ cells, plasma levels of PD-1, and/or plasma levels of PDL-1.

In other embodiments of the invention, the immune checkpoint inhibitor is an antibody to PD-1, PDL-1, or CTLA-4. In certain embodiments, the immune checkpoint inhibitor is selected from nivolumab, pembrolizumab, or ipilimumab.

In some embodiments, the CXCR4 inhibitor and immune checkpoint inhibitor act synergistically. One of ordinary skill in the art will appreciate that active agents (such as X4P-001 and an immune checkpoint inhibitor) act synergistically when the combination of active agents results in an effect that is greater than the additive effect of each agent taken separately. In some embodiments, the immune checkpoint inhibitor is nivolumab.

Other checkpoint inhibitors that may be used in the present invention include inhibitors of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

Other checkpoint inhibitors that may be used in the present invention include inhibitors of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

Checkpoint inhibitors that may be used in the present invention also include inhibitors of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Other checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Other checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Other checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Other checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Other checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Other checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Other checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Other checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Other checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Other checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-054 (LY3022855, Lilly), an anti-CSF-1R antibody, in pancreatic cancer (NCT03153410), melanoma (NCT03101254), and solid tumors (NCT02718911); and BLZ945 (4-[2((R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Other checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

Other immune-oncology agents that may be used in the present invention in combination with CXCR4 inhibitors such as X4P-001 include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

Other additional therapeutic agents that may be used in the present invention in combination with CXCR4 inhibitors such as X4P-001 include glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

### Exemplary Standard of Care Therapies

### Ovarian Cancer

In some embodiments, the present invention provides a method of treating ovarian cancer in a patient in need thereof, comprising administering to the patient an effective amount of X4P-001 optionally in combination with one or more standard of care treatments, or a combination thereof, for ovarian cancer.

Standard of care treatments for ovarian cancer are well known to one of ordinary skill in the art and include surgery, radiotherapy, or chemotherapy, or a combination thereof. In some embodiments, the standard of care chemotherapy is selected from bevacizumab, carboplatin, cisplatin, cyclophosphamide, docetaxel, doxorubicin HCl, gemcitabine HCl, megestrol acetate, melphalan, niraparib tosylate monohydrate, olaparib, paclitaxel, pemetrexed (Alimta®; Lilly), rucaparib camsylate, thiotepa, topotecan HCl, erlotinib, irinotecan, oxaliplatin, or farletuzumab (MORAb-003) (Morphotek). In some embodiments, the additional therapeutic agent is selected from niraparib (Zejula®; Tesaro), olaparib (Lynparza®; AstraZeneca), and rucaparib (Rubraca®; Clovis Onco).

In some embodiments, X4P-001 is administered to the patient as a monotherapy and as the first-line treatment for the ovarian cancer. In other embodiments, X4P-001 is administered to the patient as a first-line treatment in combination with a standard of care treatment for ovarian cancer (e.g., surgery, radiotherapy, or chemotherapy, or a combination thereof). In some embodiments, X4P-001 is administered in combination with bevacizumab and another chemotherapy.

In some embodiments, when a standard of care treatment fails, such as when surgery fails to remove all cancerous tissue or the ovarian cancer is partially resistant to a chemotherapy, a second-line treatment is used that can include a well-known second-line treatment to treat ovarian cancer. Accordingly, in some embodiments, the present invention provides a method of treating ovarian cancer in a patient wherein the cancer is resistant to a first-line therapy, said method comprising administering X4P-001 optionally in combination with a second-line treatment.

In some embodiments, the present invention provides a method of treating a resistant ovarian cancer comprising administering X4P-001 as the second-line treatment. In some embodiments, the present invention provides a method of treating a resistant ovarian cancer comprising administering X4P-001 in combination with another second-line treatment or standard of care second-line treatment for ovarian cancer (e.g., radiotherapy, chemotherapy, hormone blocking therapy, targeted immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat ovarian cancer. In some embodiments, the present invention provides a method of treating an ovarian cancer resistant to both first-line therapy and second-line therapy comprising administering X4P-001 as the third-line treatment. In some embodiments, the present invention provides a method of treating an ovarian cancer resistant to both first-line therapy and second-line therapy comprising administering X4P-001 in combination with another third-line treatment or standard of care third-line treatment for ovarian cancer (e.g., radiotherapy, chemotherapy, hormone blocking therapy, targeted immunotherapy, etc.).

In some embodiments, X4P-001 is administered as a sensitizer for the treatment of ovarian cancer. Without wishing to be bound by any particular theory, it is believed that X4P-001 increases the efficacy of the standard of care, first-line, second-line, or third-line treatments for ovarian cancer. In some embodiments, the present invention provides a method of treating an ovarian cancer in a patient in need thereof, comprising administering X4P-001 to the patient prior to administration of one or more of a standard of care, first-line, second-line, or third-line treatment. In some embodiments, administration of X4P-001 results in a more effective treatment of the ovarian cancer compared to treatment of ovarian cancer in the absence of administration of X4P-001. In some embodiments, the present invention provides a method of treating an ovarian cancer in a patient in need thereof, comprising administering X4P-001 to the patient after administration of one or more of a standard of care, first-line, second-line, or third-line treatment.

In some embodiments, the present invention provides a method of treating an ovarian cancer in a patient in need thereof, comprising administering X4P-001 to the patient in combination with an additional therapeutic agent suitable for treating the ovarian cancer. In some embodiments, the additional therapeutic agent is selected from bevacizumab, carboplatin, cisplatin, cyclophosphamide, docetaxel, doxorubicin HCl, gemcitabine HCl, megestrol acetate, melphalan, niraparib tosylate monohydrate, olaparib, paclitaxel, pemetrexed (Alimta® Lilly), rucaparib camsylate, thiotepa, topotecan HCl, erlotinib, irinotecan, oxaliplatin, or farletuzumab (MORAb-003) (Morphotek). In some embodiments, the additional therapeutic agent is selected from niraparib (Zejula®; Tesaro), olaparib (Lynparza®: AstraZeneca), and rucaparib (Rubraca®; Clovis Onco).

One of ordinary skill in the art will understand the amount and dosing regimen to administer such additional therapeutic agents for the treatment of ovarian cancer. By way of example, the administration of exemplary therapeutic agents suitable for treating ovarian cancer is summarized in Table 1, below.

TABLE 1
Exemplary Therapies for Ovarian Cancer

| Therapeutic | |
|---|---|
| Agent | Dosing regimen |
| niraparib | 300 mg once daily with or without food |
| (Zejulac ®); | until disease progression or unacceptable |
| Tesaro) | adverse reaction. For adverse |
| | reactions, consider interruption of |
| | treatment, dose reduction, |
| | or dose discontinuation. |
| olaparib | 300 mg b.i.d. with or without food until disease |
| (Lynparza ®; | progression or unacceptable toxicity. |
| AstraZeneca) | For adverse reaction, consider dose |
| | interruption or dose reduction. |
| | Reduce to 200 mg b.i.d. for moderate |
| | renal impairment (CLer 31-50 mL/min) |
| | or when co-administration with |
| | a moderate CYP3A inhibitor |
| | (e.g., aprepitant, verapamil, dilitiazem, etc.). |
| | Reduce to 150 mg b.i.d. when co-administration |
| | with strong CYP3A inhibitor (e.g., |
| | ritonavir, indinavir, nelfinavir, saquinavir, etc.) |
| rucaparib | 600 mg b.i.d. with or without food until |
| (Rubraca ®; | disease progression or unacceptable toxicity. |
| Clovis Onco) | For adverse reaction, consider interruption |
| | of treatment or dose reduction. |

### Breast Cancer

In some embodiments, the present invention provides a method of treating breast cancer in a patient in need thereof, comprising administering to the patient an effective amount of X4P-001 optionally in combination with one or more standard of care treatments, or a combination thereof, for breast cancer.

Standard of care treatments for breast cancer are well known to one of ordinary skill in the art and include surgery, radiotherapy, or chemotherapy, or a combination thereof. In some embodiments, the standard of care chemotherapy is selected from abemaciclib, anastrozole, capecitabine, cyclophosphamide, docetaxel, doxorubicin HCl, epirubicin HCl, eribulin, everolimus, exemestane, 5-fluorouracil injection, fulvestrant, gemcitabine HCl, goserelin, ixabepilone, lapatinib ditosylate, ixabepilone (BMS), letrozole, megestrol acetate, methotrexate, mitoxantrone, olaparib, paclitaxel, palbociclib, pamidronate disodium, pertuzumab, ribociclib, tamoxifen citrate, thiotepa, toremifene, trastuzumab, vinblastine, raloxifene or tamoxifen for prevention, vinorelbiine (Navelbine®; Pierre Fabre), vincristine, neratinib, and paclitaxel. In some embodiments, the additional therapeutic agent is selected from neratinib (Nerlynx®; Puma), olaparib and (Lynparza®; AstraZeneca).

In some embodiments, X4P-001 is administered to the patient as a monotherapy and as the first-line treatment for the breast cancer. In other embodiments, X4P-001 is administered to the patient as a first-line treatment in combination with a standard of care treatment for breast cancer (e.g., surgery, radiotherapy, or chemotherapy, or a combination thereof). For example, X4P-001 is administered to a patient as a first-line treatment in triple negative breast cancer in combination with a standard of care chemotherapy or CPI-613 (6,8-bis[benzylthio]octanoic acid).

In some embodiments, when a standard of care treatment fails, such as when surgery fails to remove all cancerous tissue or the breast cancer is partially resistant to a chemotherapy, a second-line treatment is used that can include a well-known second-line treatment to treat breast cancer. Accordingly, in some embodiments, the present invention provides a method of treating breast cancer in a patient wherein the cancer is resistant to a first-line therapy, said method comprising administering X4P-001 optionally in combination with a second-line treatment.

In some embodiments, the present invention provides a method of treating a resistant breast cancer comprising administering X4P-001 as the second-line treatment. In some embodiments, the present invention provides a method of treating a resistant breast cancer comprising administering X4P-001 in combination with another second-line treatment or standard of care second-line treatment for breast cancer (e.g., radiotherapy, chemotherapy, hormone blocking therapy, targeted immunotherapy, etc.). In some embodiments, the second-line treatment is selected from radiotherapy and chemotherapy.

In some instances when the first-line or second-line standard of care treatment fails, such as when chemotherapy continues to fail and remission occurs, a third-line treatment is administered to the patient that can include a well-known third-line treatment to treat breast cancer. In some embodiments, the present invention provides a method of treating a breast cancer resistant to both first-line therapy and second-line therapy comprising administering X4P-001 as the third-line treatment. In some embodiments, the present invention provides a method of treating a breast cancer resistant to both first-line therapy and second-line therapy comprising administering X4P-001 in combination with another third-line treatment or standard of care third-line treatment for breast cancer (e.g., radiotherapy, chemotherapy, hormone blocking therapy, targeted immunotherapy, etc.). For example, X4P-001 is administered as a third-line treatment or even higher in estrogen receptor positive (ER+) breast cancer alone or in combination with a chemotherapy.

In some embodiments, X4P-001 is administered as a sensitizer for the treatment of breast cancer. Without wishing to be bound by any particular theory, it is believed that X4P-001 increases the efficacy of the standard of care, first-line, second-line, or third-line treatments for breast cancer. In some embodiments, the present invention provides a method of treating a breast cancer in a patient in need thereof, comprising administering X4P-001 to the patient prior to administration of one or more of a standard of care, first-line, second-line, or third-line treatment. In some embodiments, administration of X4P-001 results in a more effective treatment of the breast cancer compared to treatment of breast cancer in the absence of administration of X4P-001. In some embodiments, the present invention provides a method of treating a breast cancer in a patient in need thereof, comprising administering X4P-001 to the patient after administration of one or more of a standard of care, first-line, second-line, or third-line treatment.

In some embodiments, the present invention provides a method of treating a breast cancer in a patient in need thereof, comprising administering X4P-001 to the patient in combination with an additional therapeutic agent suitable for treating the breast cancer. In some embodiments, the additional therapeutic agent is selected from abemaciclib, anastrozole, capecitabine, cyclophosphamide, docetaxel, doxorubicin HCl, epirubicin HCl, eribulin, everolimus, exemestane, 5-fluorouracil injection, fulvestrant, gemcitabine HCl, goserelin, ixabepilone, lapatinib ditosylate, ixabepilone (BMS), letrozole, megestrol acetate, methotrexate, mitoxantrone, olaparib, paclitaxel, palbociclib, pamidronate disodium, pertuzumab, ribociclib, tamoxifen citrate, thiotepa, toremifene, trastuzumab, vinblastine, raloxifene or tamoxifen for prevention, vinorelbiine (Navelbine®; Pierre Fabre), vincristine, neratinib, and paclitaxel. In some embodiments, the additional therapeutic agent is selected from neratinib (Nerlynx®; Puma) and olaparib (Lynparza®; AstraZeneca).

One of ordinary skill in the art will understand the amount and dosing regimen to administer such additional therapeutic agents for the treatment of breast cancer. By way of example, the administration of exemplary therapeutic agents suitable for treating breast cancer is summarized in Table 2, below.

TABLE 2
Exemplary Therapies for Breast Cancer

| Therapeutic | |
|---|---|
| Agent | Dosing regimen |
| neratinib | 240 mg once daily with food, continuously |
| (Nerlynx ®; | for one year. Dose interruptions and/or dose |
| Puma) | reductions are recommended based on |
| | individual safety and tolerability. Reduce starting |
| | dose to 80 mg in patients with severe hepatic |
| | impairment. Use loperamide with the |
| | first dose and continue during first 2 |
| | cycles (56 days) of treatment. Instruct patients |
| | to maintain 1-2 bowel movements per day and |
| | on how to use antidiarrheal treatment regimens. |
| olaparib | 300 mg b.i.d. with or without food until disease |
| (Lynparza ®; | progression or unacceptable toxicity. |
| AstraZeneca) | For adverse reaction, consider dose interruption |
| | or dose reduction. Reduce to 200 mg b.i.d. |
| | for moderate renal impairment (CLer 31-50 |
| | mL/min) or when co-administration with |
| | a moderate CYP3A inhibitor (e.g., aprepitant, |
| | verapamil, dilitiazem, etc.). Reduce to 150 mg b.i.d. |
| | when co-administration with strong CYP3A |
| | inhibitor (e.g., ritonavir, indinavir, nelfinavir, |
| | saquinavir, etc.). |

### Pancreatic Cancer

In some embodiments, the present invention provides a method of treating pancreatic cancer in a patient in need thereof, comprising administering to the patient an effective amount of X4P-001 optionally in combination with one or more standard of care treatments, or a combination thereof, for pancreatic cancer.

Standard of care treatments for pancreatic cancer are well known to one of ordinary skill in the art and include surgery, radiotherapy, or chemotherapy, or a combination thereof. In some embodiments, the standard of care chemotherapy is selected from erlotinib, everolimus, 5-fluorouracil, gemcitabine, capecitine, irinotecan HCl liposome, mitomycin C, paclitaxel albumin-stabilized, sunitinib malate, lanreotide acetate, leucovorin calcium, irinotecan hydrochloride, and oxaliplatin (FOLFIRINOX), OFF regimen (5-fluorouracil, leucovorin, and oxaliplatin), and doxorubicin. In some embodiments, the additional therapeutic agent is selected from sunitinib (Sutent®; Pfizer) and erlotinib (Tarceeva®; Genentech).

In some embodiments, X4P-001 is administered to the patient as a monotherapy and as the first-line treatment for the pancreatic cancer. In other embodiments, X4P-001 is administered to the patient as a first-line treatment in combination with a standard of care treatment for pancreatic cancer (e.g., surgery, radiotherapy, or chemotherapy, or a combination thereof). For example, X4P-001 is administered to a patient as a first-line treatment in combination with a standard of care chemotherapy.

In some embodiments, when a standard of care treatment fails, such as when surgery fails to remove all cancerous tissue or the pancreatic cancer is partially resistant to a chemotherapy, a second-line treatment is used that can include a well-known second-line treatment to treat pancreatic cancer. Accordingly, in some embodiments, the present invention provides a method of treating pancreatic cancer in a patient wherein the cancer is resistant to a first-line therapy, said method comprising administering X4P-001 optionally in combination with a second-line treatment.

In some embodiments, the present invention provides a method of treating a resistant pancreatic cancer comprising administering X4P-001 as the second-line treatment. In some embodiments, the present invention provides a method of treating a resistant pancreatic cancer comprising administering X4P-001 in combination with another second-line treatment or standard of care second-line treatment for pancreatic cancer (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat pancreatic cancer. In some embodiments, the present invention provides a method of treating a pancreatic cancer resistant to both first-line therapy and second-line therapy comprising administering X4P-001 as the third-line treatment. In some embodiments, the present invention provides a method of treating a pancreatic cancer resistant to both first-line therapy and second-line therapy comprising administering X4P-001 in combination with another third-line treatment or standard of care third-line treatment for pancreatic cancer (e.g., radiotherapy, chemotherapy, etc.).

In some embodiments, X4P-001 is administered as a sensitizer for the treatment of pancreatic cancer. Without wishing to be bound by any particular theory, it is believed that X4P-001 increases the efficacy of the standard of care, first-line, second-line, or third-line treatments for pancreatic cancer. In some embodiments, the present invention provides a method of treating a pancreatic cancer in a patient in need thereof, comprising administering X4P-001 to the patient prior to administration of one or more of a standard of care, first-line, second-line, or third-line treatment. In some embodiments, administration of X4P-001 results in a more effective treatment of the pancreatic cancer compared to treatment of pancreatic cancer in the absence of administration of X4P-001. In some embodiments, the present invention provides a method of treating a pancreatic cancer in a patient in need thereof, comprising administering X4P-001 to the patient after administration of one or more of a standard of care, first-line, second-line, or third-line treatment.

In some embodiments, the present invention provides a method of treating a pancreatic cancer in a patient in need thereof, comprising administering X4P-001 to the patient in combination with an additional therapeutic agent suitable for treating the pancreatic cancer. In some embodiments, the additional therapeutic agent is selected from erlotinib, everolimus, 5-fluorouracil, gemcitabine, capecitine, irinotecan HCl liposome, mitomycin C, paclitaxel albumin-stabilized, sunitinib malate, lanreotide acetate, leucovorin calcium, irinotecan hydrochloride, and oxaliplatin (FOLFIRINOX), OFF regimen (5-fluorouracil, leucovorin, and oxaliplatin), and doxorubicin. In some embodiments, the additional therapeutic agent is selected from sunitinib (Sutent®; Pfizer) and erlotinib (Tarceeva®; Genentech).

One of ordinary skill in the art will understand the amount and dosing regimen to administer such additional therapeutic agents for the treatment of pancreatic cancer. By way of example, the administration of exemplary therapeutic agents suitable for treating pancreatic cancer is summarized in Table 3, below.

TABLE 3
Exemplary Therapies for Pancreatic Cancer

| Therapeutic | |
|---|---|
| Agent | Dosing regimen |
| sunitinib | 50 mg orally once daily, with or |
| (Sutene ®; | without food, 4 weeks on treatment |
| Pfizer) | followed by 2 weeks off. Dose |
| | interruptions and/or dose adjustments |
| | of 12.5 mg recommended based on |
| | individual safety and tolerability. |
| erlotinib | 100 mg daily. |
| (Tarceeva ®; | Doses should be taken on an empty |
| Genentech) | stomach at least one hour before |
| | or two hours after food. |
| | Reduce in 50 mg decrements, |
| | when necessary. |

### Liver Cancer

In some embodiments, the present invention provides a method of treating liver cancer, such as but not limited to heptocarcinoma, in a patient in need thereof, comprising administering to the patient an effective amount of X4P-001 optionally in combination with one or more standard of care treatments, or a combination thereof, for liver cancer.

Standard of care treatments for liver cancer are well known to one of ordinary skill in the art and include surgery, percutaneous ablation, local chemotherapy, targeted radiotherapy, transarterial chemoembolization, or a combination thereof. In some embodiments, the standard of care chemotherapy is selected from regorafenib, sunitinib, and brivanib (BMS-582664). In some embodiments, the additional therapeutic agent is sorafenib (Nexavar®; Bayer AG and Onyx).

In some embodiments, X4P-001 is administered to the patient as a monotherapy and as the first-line treatment for the liver cancer. In other embodiments, X4P-001 is administered to the patient as a first-line treatment in combination with a standard of care treatment for liver cancer (e.g., surgery, percutaneous ablation, local chemotherapy, targeted radiotherapy, transarterial chemoembolization, or a combination thereof).

In some embodiments, when a standard of care treatment fails, such as when surgery fails to remove all cancerous tissue or the liver cancer is partially resistant to a chemotherapy, a second-line treatment is used that can include a well-known second-line treatment to treat liver cancer. Accordingly, in some embodiments, the present invention provides a method of treating liver cancer in a patient wherein the cancer is resistant to a first-line therapy, said method comprising administering X4P-001 optionally in combination with a second-line treatment.

In some embodiments, the present invention provides a method of treating a resistant liver cancer comprising administering X4P-001 as the second-line treatment. In some embodiments, the present invention provides a method of treating a resistant liver cancer comprising administering X4P-001 in combination with another second-line treatment or standard of care second-line treatment for liver cancer (e.g., radiotherapy, chemotherapy, etc.). In some embodiments, the second-line treatment is a vascular endothelial growth factor tyrosine kinase inhibitor (VEGF TKI).

In some instances when the first-line or second-line standard of care treatment fails, such as when chemotherapy continues to fail and remission occurs, a third-line treatment is administered to the patient that can include a well-known third-line treatment to treat liver cancer. In some embodiments, the present invention provides a method of treating a liver cancer resistant to both first-line therapy and second-line therapy comprising administering X4P-001 as the third-line treatment. In some embodiments, the present invention provides a method of treating a liver cancer resistant to both first-line therapy and second-line therapy comprising administering X4P-001 in combination with another third-line treatment or standard of care third-line treatment for liver cancer (e.g., radiotherapy, chemotherapy, etc.).

In some embodiments, X4P-001 is administered as a sensitizer for the treatment of liver cancer. Without wishing to be bound by any particular theory, it is believed that X4P-001 increases the efficacy of the standard of care, first-line, second-line, or third-line treatments for liver cancer. In some embodiments, the present invention provides a method of treating a liver cancer in a patient in need thereof, comprising administering X4P-001 to the patient prior to administration of one or more of a standard of care, first-line, second-line, or third-line treatment. In some embodiments, administration of X4P-001 results in a more effective treatment of the liver cancer compared to treatment of liver cancer in the absence of administration of X4P-001. In some embodiments, the present invention provides a method of treating a liver cancer in a patient in need thereof, comprising administering X4P-001 to the patient after administration of one or more of a standard of care, first-line, second-line, or third-line treatment.

In some embodiments, the present invention provides a method of treating a liver cancer in a patient in need thereof, comprising administering X4P-001 to the patient in combination with an additional therapeutic agent suitable for treating the liver cancer. In some embodiments, the additional therapeutic agent is selected from regorafenib, sun nib, and brivanib (BMS 582664). In some embodiments, the additional therapeutic agent is sorafenib (Nexavar®; Bayer AG and Onyx).

One of ordinary skill in the art will understand the amount and dosing regimen to administer such additional therapeutic agents for the treatment of liver cancer. By way of example, the administration of exemplary therapeutic agents suitable for treating liver cancer is summarized in Table 4, below.

TABLE 4
Exemplary Therapies for Liver Cancer

| Therapeutic | |
|---|---|
| Agent | Dosing regimen |
| sorafenib | 400 mg orally twice |
| (Nexavar ®; | daily without food. |
| Bayer AG | Treatment interruption and/ |
| and Onyx) | or dose reduction may be needed. |

### Waldenström's Macroglobulinemia

In some embodiments, the present invention provides a method of treating Waldenstrom's macroglobulinemia in a patient in need thereof, comprising administering to the patient an effective amount of X4P-001 optionally in combination with one or more standard of care treatments, or a combination thereof, for Waldenstrom's macroglobulinemia.

Standard of care treatments for Waldenstrom's macroglobulinemia are well known to one of ordinary skill in the art and include chemotherapy, or immunotherapy, or a combination thereof. In some embodiments, the standard of care chemotherapy is selected from chlorambucil, cladribine, cyclophosphamide, fludarabine, bendamustine, and ibrutinib. In some embodiments, the additional therapeutic agent is ibrutinib (Imbruvica®; Pharmacyclics/Janssen/AbbVie).

In some embodiments, X4P-001 is administered to the patient as a monotherapy and as the first-line treatment for the Waldenstrom's macroglobulinemia. In other embodiments, X4P-001 is administered to the patient as a first-line treatment in combination with a standard of care treatment for Waldenstrom's macroglobulinemia (e.g., immunotherapy, or chemotherapy, or a combination thereof).

In some embodiments, when a standard of care treatment fails, such as when the Waldenstrom's macroglobulinemia is partially resistant to a chemotherapy, a second-line treatment is used that can include a well-known second-line treatment to treat Waldenstrom's macroglobulinemia. Accordingly, in some embodiments, the present invention provides a method of treating Waldenstrom's macroglobulinemia in a patient wherein the cancer is resistant to a first-line therapy, said method comprising administering X4P-001 optionally in combination with a second-line treatment.

In some embodiments, the present invention provides a method of treating a resistant Waldenstrom's macroglobulinemia comprising administering X4P-001 as the second-line treatment. In some embodiments, the present invention provides a method of treating a resistant Waldenstrom's macroglobulinemia comprising administering X4P-001 in combination with another second-line treatment or standard of care second-line treatment for Waldenstrom's macroglobulinemia (e.g., immunotherapy, chemotherapy, etc.). In some embodiments, the second-line treatment is selected from a chemotherapy. For example, X4P-001 is administered as a second-line therapy in combination with a chemotherapy for the treatment of relapsed and refractory Waldenstrom's macroglobulinemia.

In some instances when the first-line or second-line standard of care treatment fails, such as when chemotherapy continues to fail and remission occurs, a third-line treatment is administered to the patient that can include a well-known third-line treatment to treat Waldenstrom's macroglobulinemia. In some embodiments, the present invention provides a method of treating a Waldenstrom's macroglobulinemia resistant to both first-line therapy and second-line therapy comprising administering X4P-001 as the third-line treatment. In some embodiments, the present invention provides a method of treating a Waldenstrom's macroglobulinemia resistant to both first-line therapy and second-line therapy comprising administering X4P-001 in combination with another third-line treatment or standard of care third-line treatment for Waldenstrom's macroglobulinemia (e.g., immunotherapy, chemotherapy, etc.).

In some embodiments, X4P-001 is administered as a sensitizer for the treatment of Waldenstrom's macroglobulinemia. Without wishing to be bound by any particular theory, it is believed that X4P-001 increases the efficacy of the standard of care, first-line, second-line, or third-line treatments for Waldenstrom's macroglobulinemia. In some embodiments, the present invention provides a method of treating a Waldenstrom's macroglobulinemia in a patient in need thereof, comprising administering X4P-001 to the patient prior to administration of one or more of a standard of care, first-line, second-line, or third-line treatment. In some embodiments, administration of X4P-001 results in a more effective treatment of the Waldenstrom's macroglobulinemia compared to treatment of Waldenstrom's macroglobulinemia in the absence of administration of X4P-001. In some embodiments, the present invention provides a method of treating a Waldenstrom's macroglobulinemia in a patient in need thereof, comprising administering X4P-001 to the patient after administration of one or more of a standard of care, first-line, second-line, or third-line treatment.

In some embodiments, the present invention provides a method of treating a Waldenstrom's macroglobulinemia in a patient in need thereof, comprising administering X4P-001 to the patient in combination with an additional therapeutic agent suitable for treating the Waldenstrom's macroglobulinemia. In some embodiments, the additional therapeutic agent is selected from chlorambucil, cladribine, cyclophosphamide, fludarabine, bendamustine, and ibrutinib. In some embodiments, the additional therapeutic agent is ibrutinib (Imbruvica®; Pharmacyclics/Janssen/AbbVie).

One of ordinary skill in the art will understand the amount and dosing regimen to administer such additional therapeutic agents for the treatment of Waldenstrom's macroglobulinemia. By way of example, the administration of exemplary therapeutic agents suitable for treating Waldenstrom's macroglobulinemia is summarized in Table 5, below.

TABLE 5
Exemplary Therapies for
Waldenström's Macroglobulinemia

| Therapeutic | |
|---|---|
| Agent | Dosing regimen |
| ibrutinib | 420 mg taken orally once daily |
| (Imbruvica ®; | for WM until disease progression |
| Pharmacyclics/ | or unacceptable toxicity. Doses |
| Janssen/AbbVie) | taken with a glass of water. |

### Head and Neck Cancer

In some embodiments, the present invention provides a method of treating head and neck cancer, such as but not limited to a squamous cell carcinoma, in a patient in need thereof, comprising administering to the patient an effective amount of X4P-001 optionally in combination with one or more standard of care treatments, or a combination thereof, for head and neck cancer.

Standard of care treatments for head and neck cancer are well known to one of ordinary skill in the art and include surgery, radiotherapy, chemotherapy, photodynamic therapy, or targeted immunotherapy, or a combination thereof. In some embodiments, the standard of care chemotherapy is selected from bleomycin, cetuximab, docetaxel, hydroxyurea, methotrexate, nivolumab, pembrolizumab, cisplatin, and 5-fluorouracil. In some embodiments, the additional therapeutic agent is selected from nivolumab (Opdivo®, BMS), pembrolizumab (Keytruda®; Merck), and cisplatin (Plantinol®; BMS) plus a radiotherapy.

In some embodiments, X4P-001 is administered to the patient as a monotherapy and as the first-line treatment for the head and neck cancer. In other embodiments, X4P-001 is administered to the patient as a first-line treatment in combination with a standard of care treatment for head and neck cancer (e.g., surgery, radiotherapy, chemotherapy, photodynamic therapy, or targeted immunotherapy, or a combination thereof). For example, X4P-001 is administered to a patient with head and neck cancer as a first-line treatment in combination with radiotherapy and cisplatin or CPI-613 (6,8-bis[benzylthio]octanoic acid).

In some embodiments, when a standard of care treatment fails, such as when surgery fails to remove all cancerous tissue or the head and neck cancer is partially resistant to a chemotherapy, a second-line treatment is used that can include a well-known second-line treatment to treat head and neck cancer. Accordingly, in some embodiments, the present invention provides a method of treating head and neck cancer in a patient wherein the cancer is resistant to a first-line therapy, said method comprising administering X4P-001 optionally in combination with a second-line treatment.

In some embodiments, the present invention provides a method of treating a resistant head and neck cancer comprising administering X4P-001 as the second-line treatment. In some embodiments, the present invention provides a method of treating a resistant head and neck cancer comprising administering X4P-001 in combination with another second-line treatment or standard of care second-line treatment for head and neck cancer (e.g., radiotherapy, chemotherapy, photodynamic therapy, or targeted immunotherapy, etc.). In some embodiments, the second-line treatment is selected from a chemotherapy. For example, X4P-001 is administered to a patient with head and neck cancer as a second-line treatment in combination with a standard of care chemotherapy or CPI-613 (6,8-bis[benzylthio]octanoic acid).

In some instances when the first-line or second-line standard of care treatment fails, such as when chemotherapy continues to fail and remission occurs, a third-line treatment is administered to the patient that can include a well-known third-line treatment to treat head and neck cancer. In some embodiments, the present invention provides a method of treating a head and neck cancer resistant to both first-line therapy and second-line therapy comprising administering X4P-001 as the third-line treatment. In some embodiments, the present invention provides a method of treating a head and neck cancer resistant to both first-line therapy and second-line therapy comprising administering X4P-001 in combination with another third-line treatment or standard of care third-line treatment for head and neck cancer (e.g., radiotherapy, chemotherapy, photodynamic therapy, or targeted immunotherapy, etc.).

In some embodiments, X4P-001 is administered as a sensitizer for the treatment of head and neck cancer. Without wishing to be bound by any particular theory, it is believed that X4P-001 increases the efficacy of the standard of care, first-line, second-line, or third-line treatments for head and neck cancer. In some embodiments, the present invention provides a method of treating a head and neck cancer in a patient in need thereof, comprising administering X4P-001 to the patient prior to administration of one or more of a standard of care, first-line, second-line, or third-line treatment. In some embodiments, administration of X4P-001 results in a more effective treatment of the head and neck cancer compared to treatment of head and neck cancer in the absence of administration of X4P-001. In some embodiments, the present invention provides a method of treating a head and neck cancer in a patient in need thereof, comprising administering X4P-001 to the patient after administration of one or more of a standard of care, first-line, second-line, or third-line treatment.

In some embodiments, the present invention provides a method of treating a head and neck cancer in a patient in need thereof, comprising administering X4P-001 to the patient in combination with an additional therapeutic agent suitable for treating the head and neck cancer. In some embodiments, the additional therapeutic agent is selected from bleomycin, cetuximab, docetaxel, hydroxyurea, methotrexate, nivolumab, pembrolizumab, cisplatin, and 5-fluorouracil. In some embodiments, the additional therapeutic agent is selected from nivolumab (Opdivo®; BMS), pembrolizumab (Keytruda®; Merck), and cisplatin (Plantinol®; BMS) plus a radiotherapy.

One of ordinary skill in the art will understand the amount and dosing regimen to administer such additional therapeutic agents, or radiotherapy, or a combination thereof, for the treatment of head and neck cancer. By way of example, the administration of exemplary therapeutic agents and radiotherapy suitable for treating head and neck cancer is summarized in Table 6, below.

TABLE 6
Exemplary Therapies for Head and Neck Cancer

| Therapeutic | |
|---|---|
| Agent | Dosing regimen |
| nivolumab | 3 mg/kg administered as an |
| (Opdivo ®; BMS) | intravenous infusion over 60 |
| | minutes every 2 weeks. |
| pembrolizurnab | 200 mg administered as an |
| (Keytruda ®; | intravenous infusion over 30 |
| Merck) | minutes every 3 weeks. |
| cisplatin | 20 to 100 mg/m² IV daily for 5 days |
| (Plantinol ®; BMS) | per cycle. 50 to 100 mg/m² IV per |
| | cycle once every 3 to 4 weeks |
| | depending on the extent of prior exposure |
| | to radiation therapy and/or prior |
| | chemotherapy. A repeat course should not |
| | be given until the serum creatinine is |
| | below 1.5 mg/100 mL, and/or the BUN is |
| | below 25 mg/100 mL. A repeat course |
| | should not be given until circulating blood |
| | elements are at an acceptable level |
| | (platelets ≥ 100,000/mm³, WBC ≥ 4000/mm³). |
| | Subsequent doses should not be given until |
| | an audiometric analysis indicates that |
| | auditory acuity is within normal limits. |
| radiotherapy | External beam radiation (EBRT or XRT) |
| | for several minutes 5 days a week |
| | for 6 to 7 weeks. Low-dose rate (LDR) |
| | brachytherapy over several days or |
| | high-dose rate (HDR) brachytherapy |
| | over several treatments at least a week apart. |

### Kidney Cancer and Renal Cancer

In some embodiments, the present invention provides a method of treating kidney cancer or renal cancer in a patient in need thereof, comprising administering to the patient an effective amount of X4P-001 optionally in combination with one or more standard of care treatments, or a combination thereof, for kidney cancer or renal cancer.

Standard of care treatments for kidney cancer or renal cancer are well known to one of ordinary skill in the art and include surgery, radiotherapy, chemotherapy, or targeted immunotherapy, or a combination thereof. In some embodiments, the standard of care chemotherapy is selected from aldesleukin, axitinib, bevacizumab, cabozantinib-S-malate, everolimus, ipilimumb, lenvatinib mesylate, nivolumab, pazopanib HCl, sorafenib tosylate, sunitinib malate, and temsirolimus. In some embodiments, the additional therapeutic agent is axitinib (Inlyta®; Pfizer).

In some embodiments, X4P-001 is administered to the patient as a monotherapy and as the first-line treatment for the kidney cancer or renal cancer. In other embodiments, X4P-001 is administered to the patient as a first-line treatment in combination with a standard of care treatment for kidney cancer or renal cancer (e.g., surgery, radiotherapy, chemotherapy, or targeted immunotherapy, or a combination thereof). For example, X4P-001 is administered as a first-line treatment in combination with CPI-613 (6,8-bis[benzylthio]octanoic acid) and a vascular endothelial growth factor tyrosine kinase inhibitor (VEGF TKI).

In some embodiments, when a standard of care treatment fails, such as when surgery fails to remove all cancerous tissue or the kidney cancer or renal cancer is partially resistant to a chemotherapy, a second-line treatment is used that can include a well-known second-line treatment to treat kidney cancer or renal cancer. Accordingly, in some embodiments, the present invention provides a method of treating kidney cancer or renal cancer in a patient wherein the cancer is resistant to a first-line therapy, said method comprising administering X4P-001 optionally in combination with a second-line treatment.

In some embodiments, the present invention provides a method of treating a resistant kidney cancer or renal cancer comprising administering X4P-001 as the second-line treatment. In some embodiments, the present invention provides a method of treating a resistant kidney cancer or renal cancer comprising administering X4P-001 in combination with another second-line treatment or standard of care second-line treatment for kidney cancer or renal cancer (e.g., radiotherapy, chemotherapy, hormone blocking therapy, targeted immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat kidney cancer or renal cancer. In some embodiments, the present invention provides a method of treating a kidney cancer or renal cancer resistant to both first-line therapy and second-line therapy comprising administering X4P-001 as the third-line treatment. In some embodiments, the present invention provides a method of treating a kidney cancer or renal cancer resistant to both first-line therapy and second-line therapy comprising administering X4P-001 in combination with another third-line treatment or standard of care third-line treatment for kidney cancer or renal cancer (e.g., radiotherapy, chemotherapy, hormone blocking therapy, targeted immunotherapy, etc.). For example, X4P-001 is administered as a third-line treatment in combination with a vascular endothelial growth factor tyrosine kinase inhibitor (VEGF TKI).

In some embodiments, X4P-001 is administered as a sensitizer for the treatment of kidney cancer or renal cancer. Without wishing to be bound by any particular theory, it is believed that X4P-001 increases the efficacy of the standard of care, first-line, second-line, or third-line treatments for kidney cancer or renal cancer. In some embodiments, the present invention provides a method of treating a kidney cancer or renal cancer in a patient in need thereof, comprising administering X4P-001 to the patient prior to administration of one or more of a standard of care, first-line, second-line, or third-line treatment. In some embodiments, administration of X4P-001 results in a more effective treatment of the kidney cancer or renal cancer compared to treatment of kidney cancer or renal cancer in the absence of administration of X4P-001. In some embodiments, the present invention provides a method of treating a kidney cancer or renal cancer in a patient in need thereof, comprising administering X4P-001 to the patient after administration of one or more of a standard of care, first-line, second-line, or third-line treatment.

In some embodiments, the present invention provides a method of treating a kidney cancer or renal cancer in a patient in need thereof, comprising administering X4P-001 to the patient in combination with an additional therapeutic agent suitable for treating the kidney cancer or renal cancer. In some embodiments, the additional therapeutic agent is selected from aldesleukin, axitinib, bevacizumab, cabozantinib-S-malate, everolimus, ipilimumb, lenvatinib mesylate, nivolumab, pazopanib HCl, sorafenib tosylate, sunitinib malate, and temsirolimus. In some embodiments, the additional therapeutic agent is axitinib (Inlyta®; Pfizer).

One of ordinary skill in the art will understand the amount and dosing regimen to administer such additional therapeutic agents for the treatment of kidney cancer or renal cancer. By way of example, the administration of exemplary therapeutic agents suitable for treating kidney cancer or renal cancer is summarized in Table 7, below.

TABLE 7
Exemplary Therapies for
Kidney Cancer or Renal Cancer

| Therapeutic | |
|---|---|
| Agent | Dosing regimen |
| axitinib | Starting dose is 5 mg orally twice |
| (Inlyta ®; | daily approximately 12 hours |
| Pfizer) | apart with or without food. |
| | Dose adjustment can be made based |
| | on individual safety and tolerability. |
| | Dose should be swallowed whole |
| | with a glass of water. Decrease |
| | starting dose by half for moderate |
| | hepatic impairment or co-admin |
| | with strong CYP3A4/5 inhibitor |
| | (e.g., ritonavir, indinavir, |
| | nelfinavir, saquinavir, etc.). |

### Adrenocortical Adenocarcinoma and Anaplastic Thyroid Cancer

In some embodiments, the present invention provides a method of treating adrenocortical adenocarcinoma or anaplastic thyroid cancer in a patient in need thereof, comprising administering to the patient an effective amount of X4P-001 optionally in combination with one or more standard of care treatments, or a combination thereof, for adrenocortical adenocarcinoma and anaplastic thyroid cancer.

Standard of care treatments for adrenocortical adenocarcinoma or anaplastic thyroid cancer are well known to one of ordinary skill in the art and include surgery, radiotherapy, or chemotherapy, or a combination thereof. In some embodiments, the standard of care chemotherapy is selected from cabozantinib-S-malate, doxorubicin HCl, lenvatinib mesylate, sorafenib tosylate, vandetanib, mitotane, cisplatin, etoposide, and eptozotocin.

In some embodiments, X4P-001 is administered to the patient as a monotherapy and as the first-line treatment for the adrenocortical adenocarcinoma or anaplastic thyroid cancer. In other embodiments, X4P-001 is administered to the patient as a first-line treatment in combination with a standard of care treatment for adrenocortical adenocarcinoma or anaplastic thyroid cancer (e.g., surgery, radiotherapy, or chemotherapy, or a combination thereof). For example, X4P-001 is administered as a first-line treatment in combination with standard of care chemotherapy.

In some embodiments, when a standard of care treatment fails, such as when surgery fails to remove all cancerous tissue or the adrenocortical adenocarcinoma or anaplastic thyroid cancer is partially resistant to a chemotherapy, a second-line treatment is used that can include a well-known second-line treatment to treat adrenocortical adenocarcinoma or anaplastic thyroid cancer. Accordingly, in some embodiments, the present invention provides a method of treating adrenocortical adenocarcinoma or anaplastic thyroid cancer in a patient wherein the cancer is resistant to a first-line therapy, said method comprising administering X4P-001 optionally in combination with a second-line treatment.

In some embodiments, the present invention provides a method of treating a resistant adrenocortical adenocarcinoma or anaplastic thyroid cancer comprising administering X4P-001 as the second-line treatment. In some embodiments, the present invention provides a method of treating a resistant adrenocortical adenocarcinoma or anaplastic thyroid cancer comprising administering X4P-001 in combination with another second-line treatment or standard of care second-line treatment for adrenocortical adenocarcinoma or anaplastic thyroid cancer (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat adrenocortical adenocarcinoma or anaplastic thyroid cancer. In some embodiments, the present invention provides a method of treating an adrenocortical adenocarcinoma or anaplastic thyroid cancer resistant to both first-line therapy and second-line therapy comprising administering X4P-001 as the third-line treatment. In some embodiments, the present invention provides a method of treating an adrenocortical adenocarcinoma or anaplastic thyroid cancer resistant to both first-line therapy and second-line therapy comprising administering X4P-001 in combination with another third-line treatment or standard of care third-line treatment for adrenocortical adenocarcinoma or anaplastic thyroid cancer (e.g., radiotherapy, chemotherapy, etc.).

In some embodiments, X4P-001 is administered as a sensitizer for the treatment of adrenocortical adenocarcinoma or anaplastic thyroid cancer. Without wishing to be bound by any particular theory, it is believed that X4P-001 increases the efficacy of the standard of care, first-line, second-line, or third-line treatments for adrenocortical adenocarcinoma or anaplastic thyroid cancer. In some embodiments, the present invention provides a method of treating an adrenocortical adenocarcinoma or anaplastic thyroid cancer in a patient in need thereof, comprising administering X4P-001 to the patient prior to administration of one or more of a standard of care, first-line, second-line, or third-line treatment. In some embodiments, administration of X4P-001 results in a more effective treatment of the adrenocortical adenocarcinoma or anaplastic thyroid cancer compared to treatment of adrenocortical adenocarcinoma or anaplastic thyroid cancer in the absence of administration of X4P-001. In some embodiments, the present invention provides a method of treating an adrenocortical adenocarcinoma or anaplastic thyroid cancer in a patient in need thereof, comprising administering X4P-001 to the patient after administration of one or more of a standard of care, first-line, second-line, or third-line treatment.

In some embodiments, the present invention provides a method of treating an adrenocortical adenocarcinoma or anaplastic thyroid cancer in a patient in need thereof, comprising administering X4P-001 to the patient in combination with an additional therapeutic agent suitable for treating the adrenocortical adenocarcinoma or anaplastic thyroid cancer. In some embodiments, the additional therapeutic agent is selected from cabozantinib-S-malate, doxorubicin HCl, lenvatinib mesylate, sorafenib tosylate, vandetanib, mitotane, cisplatin, etoposide, and eptozotocin. One of ordinary skill in the art will understand the amount and dosing regimen to administer such additional therapeutic agents for the treatment of adrenocortical adenocarcinoma or anaplastic thyroid cancer.

### Cholangiocarcinoma (Bile Duct Cancer)

In some embodiments, the present invention provides a method of treating cholangiocarcinoma cancer in a patient in need thereof, comprising administering to the patient an effective amount of X4P-001 optionally in combination with one or more standard of care treatments, or a combination thereof, for cholangiocarcinoma.

Standard of care treatments for cholangiocarcinoma are well known to one of ordinary skill in the art and include surgery, radiotherapy, or chemotherapy, or a combination thereof. In some embodiments, the standard of care chemotherapy is selected from gemcitabine HCl (off-label), fluoropyrimidines, platinum agents, docetaxel+radiation, mitomycin-C, and 5-fluorouracil. In some embodiments, the additional therapeutic agent is gemcitabine HCl (Gemzar®; Lilly).

In some embodiments, X4P-001 is administered to the patient as a monotherapy and as the first-line treatment for the cholangiocarcinoma. In other embodiments, X4P-001 is administered to the patient as a first-line treatment in combination with a standard of care treatment for cholangiocarcinoma (e.g., surgery, radiotherapy, or chemotherapy, or a combination thereof). For example, X4P-001 is administered as a first-line treatment in combination with CPI-613 (6,8-bis[benzylthio]octanoic acid).

In some embodiments, when a standard of care treatment fails, such as when surgery fails to remove all cancerous tissue or the cholangiocarcinoma is partially resistant to a chemotherapy, a second-line treatment is used that can include a well-known second-line treatment to treat cholangiocarcinoma. Accordingly, in some embodiments, the present invention provides a method of treating cholangiocarcinoma in a patient wherein the cancer is resistant to a first-line therapy, said method comprising administering X4P-001 optionally in combination with a second-line treatment.

In some embodiments, the present invention provides a method of treating a resistant cholangiocarcinoma comprising administering X4P-001 as the second-line treatment. In some embodiments, the present invention provides a method of treating a resistant cholangiocarcinoma comprising administering X4P-001 in combination with another second-line treatment or standard of care second-line treatment for cholangiocarcinoma (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat cholangiocarcinoma. In some embodiments, the present invention provides a method of treating a cholangiocarcinoma resistant to both first-line therapy and second-line therapy comprising administering X4P-001 as the third-line treatment. In some embodiments, the present invention provides a method of treating a cholangiocarcinoma resistant to both first-line therapy and second-line therapy comprising administering X4P-001 in combination with another third-line treatment or standard of care third-line treatment for cholangiocarcinoma (e.g., radiotherapy, chemotherapy, etc.).

In some embodiments, X4P-001 is administered as a sensitizer for the treatment of cholangiocarcinoma. Without wishing to be bound by any particular theory, it is believed that X4P-001 increases the efficacy of the standard of care, first-line, second-line, or third-line treatments for cholangiocarcinoma. In some embodiments, the present invention provides a method of treating a cholangiocarcinoma in a patient in need thereof, comprising administering X4P-001 to the patient prior to administration of one or more of a standard of care, first-line, second-line, or third-line treatment. In some embodiments, administration of X4P-001 results in a more effective treatment of the cholangiocarcinoma compared to treatment of cholangiocarcinoma in the absence of administration of X4P-001. In some embodiments, the present invention provides a method of treating a cholangiocarcinoma in a patient in need thereof, comprising administering X4P-001 to the patient after administration of one or more of a standard of care, first-line, second-line, or third-line treatment.

In some embodiments, the present invention provides a method of treating a cholangiocarcinoma in a patient in need thereof, comprising administering X4P-001 to the patient in combination with an additional therapeutic agent suitable for treating the cholangiocarcinoma. In some embodiments, the additional therapeutic agent is selected from gemcitabine HCl (off-label), fluoropyrimidines, platinum agents, docetaxel+radiation, mitomycin-C, and 5-fluorouracil. In some embodiments, the additional therapeutic agent is gemcitabine HCl (Gemzar®; Lilly).

One of ordinary skill in the art will understand the amount and dosing regimen to administer such additional therapeutic agents for the treatment of cholangiocarcinoma. By way of example, the administration of exemplary therapeutic agents suitable for treating cholangiocarcinoma is summarized in Table 8, below.

TABLE 8
Exemplary Therapies for Cholangiocarcinoma

| Therapeutic | |
|---|---|
| Agent | Dosing regimen |
| gemcitabine HCl | 1000-1250 mg/m² over 30 minutes on |
| (Gemzar ®; | Days 1 and 8 of each 21-day cycle. |
| Lilly) | 4-Week schedule: 1000 mg/m² over |
| | 30 minutes on Days 1, 8, and |
| | 15 of each 28-day cycle. |
| | 3-Week schedule: 1250 mg/m² over |
| | 30 minutes on Days 1 and 8 of |
| | each 21-day cycle. |
| | 1000 mg/m² over 30 minutes once |
| | weekly for up to 7 weeks (or |
| | until toxicity necessitates reducing or |
| | holding a dose), followed by a |
| | week of rest from treatment. |
| | Subsequent cycles should consist of |
| | infusions once weekly for 3 consecutive |
| | weeks out of every 4 weeks. |
| | Dose reductions or discontinuation |
| | may be needed based on |
| | toxicities. |

### Cervical Cancer, Endometrial Cancer, and Uterine Sarcoma

In some embodiments, the present invention provides a method of treating cervical cancer, endometrial cancer, or uterine sarcoma in a patient in need thereof, comprising administering to the patient an effective amount of X4P-001 optionally in combination with one or more standard of care treatments, or a combination thereof, for cervical cancer, endometrial cancer, or uterine sarcoma.

Standard of care treatments for cervical cancer, endometrial cancer, or uterine sarcoma are well known to one of ordinary skill in the art and include surgery, radiotherapy, or chemotherapy, or a combination thereof. In some embodiments, the standard of care chemotherapy is selected from bevacizumab (Avastin®), bisplatin, 5-fluorouracil, barboplatin, paclitaxel (Taxol®), tamoxifen, topotecan, gemcitabine (Gemzar®), PI3K/mTOR inhibitor, everolimus (Afinitor®; Novartis), temsirolimus (Torisel®; Pfizer), and sirolimus (Rapamune®; Pfizer), letrozole (Femara®, Novartis), progestin hormone therapy (hydroxyprogesterone, medroxyprogesterone, and megestrol), and metformin. In some embodiments, the additional therapeutic agent is cisplatin (Plantinol®; BMS) and radiotherapy.

In some embodiments, X4P-001 is administered to the patient as a monotherapy and as the first-line treatment for the cervical cancer, endometrial cancer, or uterine sarcoma. In other embodiments, X4P-001 is administered to the patient as a first-line treatment in combination with a standard of care treatment for cervical cancer, endometrial cancer, or uterine sarcoma (e.g., surgery, radiotherapy, or chemotherapy, or a combination thereof).

In some embodiments, when a standard of care treatment fails, such as when surgery fails to remove all cancerous tissue or the cervical cancer, endometrial cancer, or uterine sarcoma is partially resistant to a chemotherapy, a second-line treatment is used that can include a well-known second-line treatment to treat cervical cancer, endometrial cancer, or uterine sarcoma. Accordingly, in some embodiments, the present invention provides a method of treating cervical cancer, endometrial cancer, or uterine sarcoma in a patient wherein the cancer is resistant to a first-line therapy, said method comprising administering X4P-001 optionally in combination with a second-line treatment.

In some embodiments, the present invention provides a method of treating a resistant cervical cancer, endometrial cancer, or uterine sarcoma comprising administering X4P-001 as the second-line treatment. In some embodiments, the present invention provides a method of treating a resistant cervical cancer, endometrial cancer, or uterine sarcoma comprising administering X4P-001 in combination with another second-line treatment or standard of care second-line treatment for cervical cancer, endometrial cancer, or uterine sarcoma (e.g., radiotherapy, chemotherapy, etc.). In some embodiments, the second-line treatment is concurrent chemoradiation. For example, X4P-001 is administered as a second-line treatment in combination with cisplatin and radiotherapy.

In some instances when the first-line or second-line standard of care treatment fails, such as when chemotherapy continues to fail and remission occurs, a third-line treatment is administered to the patient that can include a well-known third-line treatment to treat cervical cancer, endometrial cancer, or uterine sarcoma. In some embodiments, the present invention provides a method of treating a cervical cancer, endometrial cancer, or uterine sarcoma resistant to both first-line therapy and second-line therapy comprising administering X4P-001 as the third-line treatment. In some embodiments, the present invention provides a method of treating a cervical cancer, endometrial cancer, or uterine sarcoma resistant to both first-line therapy and second-line therapy comprising administering X4P-001 in combination with another third-line treatment or standard of care third-line treatment for cervical cancer, endometrial cancer, or uterine sarcoma (e.g., radiotherapy, chemotherapy, etc.).

In some embodiments, X4P-001 is administered as a sensitizer for the treatment of cervical cancer, endometrial cancer, or uterine sarcoma. Without wishing to be bound by any particular theory, it is believed that X4P-001 increases the efficacy of the standard of care, first-line, second-line, or third-line treatments for cervical cancer, endometrial cancer, or uterine sarcoma. In some embodiments, the present invention provides a method of treating a cervical cancer, endometrial cancer, or uterine sarcoma in a patient in need thereof, comprising administering X4P-001 to the patient prior to administration of one or more of a standard of care, first-line, second-line, or third-line treatment. In some embodiments, administration of X4P-001 results in a more effective treatment of the cervical cancer, endometrial cancer, or uterine sarcoma compared to treatment of cervical cancer, endometrial cancer, or uterine sarcoma in the absence of administration of X4P-001. In some embodiments, the present invention provides a method of treating a cervical cancer, endometrial cancer, or uterine sarcoma in a patient in need thereof, comprising administering X4P-001 to the patient after administration of one or more of a standard of care, first-line, second-line, or third-line treatment.

In some embodiments, the present invention provides a method of treating a cervical cancer, endometrial cancer, or uterine sarcoma in a patient in need thereof, comprising administering X4P-001 to the patient in combination with an additional therapeutic agent suitable for treating the cervical cancer, endometrial cancer, or uterine sarcoma. In some embodiments, the additional therapeutic agent is selected from bevacizumab (Avastin®), bisplatin, 5-fluorouracil, barboplatin, paclitaxel (Taxol®), tamoxifen, topotecan, gemcitabine (Gemzar®), PI3K/mTOR inhibitor, everolimus (Afinitor®; Novartis), temsirolimus (Torisel®; Pfizer), and sirolimus (Rapamune®; Pfizer), letrozole (Femara®, Novartis), progestin hormone therapy (hydroxyprogesterone, medroxyprogesterone, and megestrol), and metformin. In some embodiments, the additional therapeutic agent is cisplatin (Plantinol®; BMS) and radiotherapy.

One of ordinary skill in the art will understand the amount and dosing regimen to administer such additional therapeutic agents for the treatment of cervical cancer. By way of example, the administration of exemplary therapeutic agents and radiotherapy suitable for treating cervical cancer is summarized in Table 9, below.

TABLE 9
Exemplary Therapies for Cervical Cancer

| Therapeutic | |
|---|---|
| Agent | Dosing regimen |
| cisplatin | 20 to 100 mg/m² IV daily for 5 days |
| (Plantinol ®; | per cycle. 50 to 100 mg/m² IV per cycle |
| BMS) | once every 3 to 4 weeks depending |
| | on the extent of prior exposure |
| | to radiation therapy and/or prior |
| | chemotherapy. A repeat course should |
| | not be given until the serum creatinine |
| | is below 1.5 mg/100 mL, and/or the |
| | BUN is below 25 mg/100 mL. |
| | A repeat course should not be given |
| | until circulating blood elements are at an |
| | acceptable level (platelets ≥ 100,000/mm³, |
| | WBC ≥ 4000/mm³). Subsequent doses |
| | should not be given until an audiometric |
| | analysis indicates that auditory acuity |
| | is within normal limits. |
| radiotherapy | External beam radiation for several |
| | minutes 5 days a week for 6 to 7 weeks. |
| | Low-dose rate (LDR) brachytherapy |
| | over several days or high-dose rate (HDR) |
| | brachytherapy over several treatments |
| | at least a week apart. |

### Soft Tissue Sarcoma and Bone Sarcoma

In some embodiments, the present invention provides a method of treating soft tissue sarcoma or bone sarcoma in a patient in need thereof, comprising administering to the patient an effective amount of X4P-001 optionally in combination with one or more standard of care treatments, or a combination thereof, for soft tissue sarcoma or bone sarcoma.

Standard of care treatments for soft tissue sarcoma or bone sarcoma are well known to one of ordinary skill in the art and include surgery, radiotherapy, or chemotherapy, or a combination thereof. In some embodiments, the standard of care chemotherapy for soft tissue sarcoma or bone sarcoma is selected from ifosfamide (Ifex® Baxter Healthcare alkylating agent), high-dose methotrexate, doxorubicin, docetaxel, cisplatin, high-dose ifosfamide, etoposide, carboplatin, cyclophosphamide, sorafenib, and everolimus.

In some embodiments, X4P-001 is administered to the patient as a monotherapy and as the first-line treatment for the soft tissue sarcoma or bone sarcoma. In other embodiments, X4P-001 is administered to the patient as a first-line treatment in combination with a standard of care treatment for soft tissue sarcoma or bone sarcoma (e.g., surgery, radiotherapy, or chemotherapy, or a combination thereof).

In some embodiments, when a standard of care treatment fails, such as when surgery fails to remove all cancerous tissue or the soft tissue sarcoma or bone sarcoma is partially resistant to a chemotherapy, a second-line treatment is used that can include a well-known second-line treatment to treat soft tissue sarcoma or bone sarcoma. Accordingly, in some embodiments, the present invention provides a method of treating soft tissue sarcoma or bone sarcoma in a patient wherein the cancer is resistant to a first-line therapy, said method comprising administering X4P-001 optionally in combination with a second-line treatment.

In some embodiments, the present invention provides a method of treating a resistant soft tissue sarcoma or bone sarcoma comprising administering X4P-001 as the second-line treatment. In some embodiments, the present invention provides a method of treating a resistant soft tissue sarcoma or bone sarcoma comprising administering X4P-001 in combination with another second-line treatment or standard of care second-line treatment for soft tissue sarcoma or bone sarcoma (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat soft tissue sarcoma or bone sarcoma. In some embodiments, the present invention provides a method of treating a soft tissue sarcoma or bone sarcoma resistant to both first-line therapy and second-line therapy comprising administering X4P-001 as the third-line treatment. In some embodiments, the present invention provides a method of treating a soft tissue sarcoma or bone sarcoma resistant to both first-line therapy and second-line therapy comprising administering X4P-001 in combination with another third-line treatment or standard of care third-line treatment for soft tissue sarcoma or bone sarcoma (e.g., radiotherapy, chemotherapy, etc.).

In some embodiments, X4P-001 is administered as a sensitizer for the treatment of soft tissue sarcoma or bone sarcoma. Without wishing to be bound by any particular theory, it is believed that X4P-001 increases the efficacy of the standard of care, first-line, second-line, or third-line treatments for soft tissue sarcoma or bone sarcoma. In some embodiments, the present invention provides a method of treating a soft tissue sarcoma or bone sarcoma in a patient in need thereof, comprising administering X4P-001 to the patient prior to administration of one or more of a standard of care, first-line, second-line, or third-line treatment. In some embodiments, administration of X4P-001 results in a more effective treatment of the soft tissue sarcoma or bone sarcoma compared to treatment of soft tissue sarcoma or bone sarcoma in the absence of administration of X4P-001. In some embodiments, the present invention provides a method of treating a soft tissue sarcoma or bone sarcoma in a patient in need thereof, comprising administering X4P-001 to the patient after administration of one or more of a standard of care, first-line, second-line, or third-line treatment.

In some embodiments, the present invention provides a method of treating a soft tissue sarcoma or bone sarcoma in a patient in need thereof, comprising administering X4P-001 to the patient in combination with an additional therapeutic agent suitable for treating the soft tissue sarcoma or bone sarcoma. In some embodiments, the additional therapeutic agent is selected from ifosfamide (Ifex® Baxter Healthcare alkylating agent), high-dose methotrexate, doxorubicin, docetaxel, cisplatin, high-dose ifosfamide, etoposide, carboplatin, cyclophosphamide, sorafenib, and everolimus. One of ordinary skill in the art will understand the amount and dosing regimen to administer such additional therapeutic agents for the treatment of soft tissue sarcoma or bone sarcoma.

### Glioblastoma and Other CNS Tumors

In some embodiments, the present invention provides a method of treating glioblastoma or other CNS tumors in a patient in need thereof, comprising administering to the patient an effective amount of X4P-001 optionally in combination with one or more standard of care treatments, or a combination thereof, for glioblastoma or other CNS tumors.

Standard of care treatments for glioblastoma or other CNS tumors are well known to one of ordinary skill in the art and include surgery, radiotherapy, or chemotherapy, or a combination thereof. In some embodiments, the standard of care chemotherapy is selected from bevacizumab, irinotecan, implanted carmustine wafers, procarbazine, and BRAF kinase inhibitors.

In some embodiments, X4P-001 is administered to the patient as a monotherapy and as the first-line treatment for the glioblastoma or other CNS tumors. In other embodiments, X4P-001 is administered to the patient as a first-line treatment in combination with a standard of care treatment for glioblastoma or other CNS tumors (e.g., surgery, radiotherapy, or chemotherapy, or a combination thereof). In some embodiments, X4P-001 is administered as a first-line treatment in combination with a radiotherapy.

In some embodiments, when a standard of care treatment fails, such as when surgery fails to remove all cancerous tissue or the glioblastoma or other CNS tumors are partially resistant to a chemotherapy, a second-line treatment is used that can include a well-known second-line treatment to treat glioblastoma or other CNS tumors. Accordingly, in some embodiments, the present invention provides a method of treating glioblastoma or other CNS tumors in a patient wherein the cancer is resistant to a first-line therapy, said method comprising administering X4P-001 optionally in combination with a second-line treatment.

In some embodiments, the present invention provides a method of treating a resistant glioblastoma or other CNS tumors comprising administering X4P-001 as the second-line treatment. In some embodiments, the present invention provides a method of treating a resistant glioblastoma or other CNS tumors comprising administering X4P-001 in combination with another second-line treatment or standard of care second-line treatment for glioblastoma or other CNS tumors (e.g., radiotherapy, chemotherapy, etc.). In some embodiments, the second-line treatment for relapsed glioblastoma or other CNS tumors is selected from a radiotherapy.

In some instances when the first-line or second-line standard of care treatment fails, such as when chemotherapy continues to fail and remission occurs, a third-line treatment is administered to the patient that can include a well-known third-line treatment to treat glioblastoma or other CNS tumors. In some embodiments, the present invention provides a method of treating a glioblastoma or other CNS tumors resistant to both first-line therapy and second-line therapy comprising administering X4P-001 as the third-line treatment. In some embodiments, the present invention provides a method of treating a glioblastoma or other CNS tumors resistant to both first-line therapy and second-line therapy comprising administering X4P-001 in combination with another third-line treatment or standard of care third-line treatment for glioblastoma or other CNS tumors (e.g., radiotherapy, chemotherapy, etc.).

In some embodiments, X4P-001 is administered as a sensitizer for the treatment of glioblastoma or other CNS tumors. Without wishing to be bound by any particular theory, it is believed that X4P-001 increases the efficacy of the standard of care, first-line, second-line, or third-line treatments for glioblastoma or other CNS tumors. In some embodiments, the present invention provides a method of treating a glioblastoma or other CNS tumors in a patient in need thereof, comprising administering X4P-001 to the patient prior to administration of one or more of a standard of care, first-line, second-line, or third-line treatment. In some embodiments, administration of X4P-001 results in a more effective treatment of the glioblastoma or other CNS tumors compared to treatment of glioblastoma or other CNS tumors in the absence of administration of X4P-001. In some embodiments, the present invention provides a method of treating a glioblastoma or other CNS tumors in a patient in need thereof, comprising administering X4P-001 to the patient after administration of one or more of a standard of care, first-line, second-line, or third-line treatment.

In some embodiments, the present invention provides a method of treating a glioblastoma or other CNS tumors in a patient in need thereof, comprising administering X4P-001 to the patient in combination with an additional therapeutic agent suitable for treating the glioblastoma or other CNS tumors. In some embodiments, the additional therapeutic agent is selected from bevacizumab, irinotecan, implanted carmustine wafers, procarbazine, and BRAF kinase inhibitors. One of ordinary skill in the art will understand the amount and dosing regimen to administer such additional therapeutic agents for the treatment of glioblastoma or other CNS tumors.

### Lung Cancer

In some embodiments, the present invention provides a method of treating lung cancer, such as but not limited to non-small-cell lung carcinoma (NSCLC), in a patient in need thereof, comprising administering to the patient an effective amount of X4P-001 optionally in combination with one or more standard of care treatments, or a combination thereof, for lung cancer.

Standard of care treatments for lung cancer are well known to one of ordinary skill in the art and include surgery, radiotherapy, chemotherapy, or targeted immunotherapy, or a combination thereof. In some embodiments, the standard of care chemotherapy is selected from afatinib dimaleate, alectinib, atezolizumab, bevacizumab, brigantinib, carboplatin, ceritinib, crizotinib, dabrafenib, docetaxel, erlotinib HCl, everolimus, gefitinib, gemcitabine HCl, mechlorethamine HCl, methotrexate, necitumumab, nivolumab, osimertinib, paclitaxel, pembrolizumab, pemetrexed disodium, ramucirumab, sunitinib, trametinib, vinorelbine tartrate (Navelbine®; Pierre Fabre), doxorubicin HCl, etoposide, and topotecan HCl. In some embodiments, the additional therapeutic agent is selected from alectinib (Alecenza®; Genentech), crizotinib (Xalkori®; Pfizer), ceritinib (Zykadia®; Novartis).

In some embodiments, X4P-001 is administered to the patient as a monotherapy and as the first-line treatment for the lung cancer. In other embodiments, X4P-001 is administered to the patient as a first-line treatment in combination with a standard of care treatment for lung cancer (e.g., surgery, radiotherapy, chemotherapy, or targeted immunotherapy, or a combination thereof).

In some embodiments, when a standard of care treatment fails, such as when surgery fails to remove all cancerous tissue or the lung cancer is partially resistant to a chemotherapy, a second-line treatment is used that can include a well-known second-line treatment to treat lung cancer. Accordingly, in some embodiments, the present invention provides a method of treating lung cancer in a patient wherein the cancer is resistant to a first-line therapy, said method comprising administering X4P-001 optionally in combination with a second-line treatment.

In some embodiments, the present invention provides a method of treating a resistant lung cancer comprising administering X4P-001 as the second-line treatment. In some embodiments, the present invention provides a method of treating a resistant lung cancer comprising administering X4P-001 in combination with another second-line treatment or standard of care second-line treatment for lung cancer (e.g., radiotherapy, chemotherapy, targeted immunotherapy, etc.). In some embodiments, the second-line treatment is selected from radiotherapy and chemotherapy.

In some instances when the first-line or second-line standard of care treatment fails, such as when chemotherapy continues to fail and remission occurs, a third-line treatment is administered to the patient that can include a well-known third-line treatment to treat lung cancer. In some embodiments, the present invention provides a method of treating a lung cancer resistant to both first-line therapy and second-line therapy comprising administering X4P-001 as the third-line treatment. In some embodiments, the present invention provides a method of treating a lung cancer resistant to both first-line therapy and second-line therapy comprising administering X4P-001 in combination with another third-line treatment or standard of care third-line treatment for lung cancer (e.g., radiotherapy, chemotherapy, targeted immunotherapy, etc.).

In some embodiments, X4P-001 is administered as a sensitizer for the treatment of lung cancer. Without wishing to be bound by any particular theory, it is believed that X4P-001 increases the efficacy of the standard of care, first-line, second-line, or third-line treatments for lung cancer. In some embodiments, the present invention provides a method of treating a lung cancer in a patient in need thereof, comprising administering X4P-001 to the patient prior to administration of one or more of a standard of care, first-line, second-line, or third-line treatment. In some embodiments, administration of X4P-001 results in a more effective treatment of the lung cancer compared to treatment of lung cancer in the absence of administration of X4P-001. In some embodiments, the present invention provides a method of treating a lung cancer in a patient in need thereof, comprising administering X4P-001 to the patient after administration of one or more of a standard of care, first-line, second-line, or third-line treatment.

In some embodiments, the present invention provides a method of treating a lung cancer in a patient in need thereof, comprising administering X4P-001 to the patient in combination with an additional therapeutic agent suitable for treating the lung cancer. In some embodiments, the additional therapeutic agent is selected from afatinib dimaleate, alectinib, atezolizumab, bevacizumab, brigantinib, carboplatin, ceritinib, crizotinib, dabrafenib, docetaxel, erlotinib HCl, everolimus, gefitinib, gemcitabine HCl, mechlorethamine HCl, methotrexate, necitumumab, nivolumab, osimertinib, paclitaxel, pembrolizumab, pemetrexed disodium, ramucirumab, sunitinib, trametinib, vinorelbine tartrate (Navelbine®; Pierre Fabre), doxorubicin HCl, etoposide, and topotecan HCl. In some embodiments, the additional therapeutic agent is selected from alectinib (Alecenza®; Genentech), crizotinib (Xalkori®; Pfizer), ceritinib (Zykadia®; Novartis).

One of ordinary skill in the art will understand the amount and dosing regimen to administer such additional therapeutic agents for the treatment of lung cancer. By way of example, the administration of exemplary therapeutic agents suitable for treating lung cancer is summarized in Table 10, below.

TABLE 10
Exemplary Therapies for Lung Cancer

| Therapeutic | |
|---|---|
| Agent | Dosing regimen |
| alectinib | 600 mg orally b.i.d. with food until |
| (Alecenza ®; | disease progression or unacceptable toxicity. |
| Genentech) | Dose reduction by 150 mg or 300 mg may |
| | be required on individual tolerability. |
| crizotinib | 250 mg taken orally b.i.d. with or without |
| (Xalkori ®; | food. Dose interruption and/or dose |
| Pfizer) | reduction to 200 mg taken orally |
| | b.i.d. may be required based on individual |
| | safety and tolerability, then to 250 mg |
| | taken orally once daily if further |
| | reduction is necessary |
| ceritinib | 450 mg orally once daily with food. |
| (Zykadia ®; | |
| Novartis) | |

### Melanoma

In some embodiments, the present invention provides a method of treating melanoma in a patient in need thereof, comprising administering to the patient an effective amount of X4P-001 optionally in combination with one or more standard of care treatments, or a combination thereof, for melanoma.

Standard of care treatments for melanoma are well known to one of ordinary skill in the art and include surgery, radiotherapy, or chemotherapy, or a combination thereof. In some embodiments, the standard of care chemotherapy is selected from aldesleukin, cobimetinib, dabrafenib, dacarbazine, ipilimumab, nivolumab, peg-interferon-alfa-2b, pembrolizumab, rec-interferon-alfa-2b, talimogene laherparepvec, trametinib, and vemurafenib. In some embodiments, the additional therapeutic agent is selected from dabrafenib (Tafinlar®; Novartis), pembrolizumab (Keytruda®; Merck).

In some embodiments, X4P-001 is administered to the patient as a monotherapy and as the first-line treatment for the melanoma. In other embodiments, X4P-001 is administered to the patient as a first-line treatment in combination with a standard of care treatment for melanoma (e.g., surgery, radiotherapy, or chemotherapy, such as pemetrexed, or a combination thereof).

In some embodiments, when a standard of care treatment fails, such as when surgery fails to remove all cancerous tissue or the melanoma is partially resistant to a chemotherapy, a second-line treatment is used that can include a well-known second-line treatment to treat melanoma. Accordingly, in some embodiments, the present invention provides a method of treating melanoma in a patient wherein the cancer is resistant to a first-line therapy, said method comprising administering X4P-001 optionally in combination with a second-line treatment.

In some embodiments, the present invention provides a method of treating a resistant melanoma comprising administering X4P-001 as the second-line treatment. In some embodiments, the present invention provides a method of treating a resistant melanoma comprising administering X4P-001 in combination with another second-line treatment or standard of care second-line treatment for melanoma (e.g., radiotherapy, chemotherapy, targeted immunotherapy, etc.). In some embodiments, the second-line treatment is selected from radiotherapy and chemotherapy.

In some instances when the first-line or second-line standard of care treatment fails, such as when chemotherapy continues to fail and remission occurs, a third-line treatment is administered to the patient that can include a well-known third-line treatment to treat melanoma. In some embodiments, the present invention provides a method of treating a melanoma resistant to both first-line therapy and second-line therapy comprising administering X4P-001 as the third-line treatment. In some embodiments, the present invention provides a method of treating a melanoma resistant to both first-line therapy and second-line therapy comprising administering X4P-001 in combination with another third-line treatment or standard of care third-line treatment for melanoma (e.g., radiotherapy, chemotherapy, targeted immunotherapy, etc.).

In some embodiments, X4P-001 is administered as a sensitizer for the treatment of melanoma. Without wishing to be bound by any particular theory, it is believed that X4P-001 increases the efficacy of the standard of care, first-line, second-line, or third-line treatments for melanoma. In some embodiments, the present invention provides a method of treating a melanoma in a patient in need thereof, comprising administering X4P-001 to the patient prior to administration of one or more of a standard of care, first-line, second-line, or third-line treatment. In some embodiments, administration of X4P-001 results in a more effective treatment of the melanoma compared to treatment of melanoma in the absence of administration of X4P-001. In some embodiments, the present invention provides a method of treating a melanoma in a patient in need thereof, comprising administering X4P-001 to the patient after administration of one or more of a standard of care, first-line, second-line, or third-line treatment.

In some embodiments, the present invention provides a method of treating a melanoma in a patient in need thereof, comprising administering X4P-001 to the patient in combination with an additional therapeutic agent suitable for treating the melanoma. In some embodiments, the additional therapeutic agent is selected from aldesleukin, cobimetinib, dabrafenib, dacarbazine, ipilimumab, nivolumab, peg-interferon-alfa-2b, pembrolizumab, rec-interferon-alfa-2b, talimogene laherparepvec, trametinib, and vemurafenib. In some embodiments, the additional therapeutic agent is selected from dabrafenib (Tafinlar®; Novartis), pembrolizumab (Keytruda®; Merck).

One of ordinary skill in the art will understand the amount and dosing regimen to administer such additional therapeutic agents for the treatment of melanoma. By way of example, the administration of exemplary therapeutic agents suitable for treating melanoma is summarized in Table 11, below.

TABLE 11
Exemplary Therapies for Melanoma

| Therapeutic | |
|---|---|
| Agent | Dosing regimen |
| dabrafenib | 150 mg orally b.i.d. at least 1 hour |
| (Tafinlar ®; | before or at least 2 hours after a meal |
| Novartis) | |
| pembrolizumab | 2 mg/kg every 3 weeks. |
| (Keytruda ®; | Administer as an intravenous |
| Merck) | infusion over 30 minutes. |

### Acute Lymphoblastic Leukemia (ALL)

In some embodiments, the present invention provides a method of treating acute lymphoblastic leukemia in a patient in need thereof, comprising administering to the patient an effective amount of X4P-001 optionally in combination with one or more standard of care treatments, or a combination thereof, for acute lymphoblastic leukemia.

Standard of care treatments for acute lymphoblastic leukemia are well known to one of ordinary skill in the art and include chemotherapy, steroids, bone marrow transplant, or stem cell transplant, or a combination thereof. In some embodiments, the standard of care chemotherapy is selected from methotrexate, nelarabine, asparaginase, inotuzumab ozogamicin, blinatumomab, daunorubicin HCl, cyclophosphamide, clorafabine, cytarabine, dasatinib, imatinib mesylate, ponatinib, ti sagenlecleucel, vincristine, mercaptopurine, pegaspargase, and prednisone. In some embodiments, the additional therapeutic agent is selected from inotuzumab ozogamicin (Besponsa®; Pfizer), imatinib mesylate, (Gleevec®; Novartis), blinatumomab (Blincyto®; Amgen), and Dasatinib (Sprycel®; BMS).

In some embodiments, X4P-001 is administered to the patient as a monotherapy and as the first-line treatment for the acute lymphoblastic leukemia. In other embodiments, X4P-001 is administered to the patient as a first-line treatment in combination with a standard of care treatment for acute lymphoblastic leukemia (e.g., chemotherapy, steroids, bone marrow transplant, or stem cell transplant, or a combination thereof).

In some embodiments, when a standard of care treatment fails, such as when the acute lymphoblastic leukemia is partially resistant to a chemotherapy, a second-line treatment is used that can include a well-known second-line treatment to treat acute lymphoblastic leukemia. Accordingly, in some embodiments, the present invention provides a method of treating acute lymphoblastic leukemia in a patient wherein the cancer is resistant to a first-line therapy, said method comprising administering X4P-001 optionally in combination with a second-line treatment.

In some embodiments, the present invention provides a method of treating a resistant acute lymphoblastic leukemia comprising administering X4P-001 as the second-line treatment. In some embodiments, the present invention provides a method of treating a resistant acute lymphoblastic leukemia comprising administering X4P-001 in combination with another second-line treatment or standard of care second-line treatment for acute lymphoblastic leukemia (e.g., radiotherapy, chemotherapy, immunotherapy, etc.). In some embodiments, the second-line treatment is a chemotherapy.

In some instances when the first-line or second-line standard of care treatment fails, such as when chemotherapy continues to fail and remission occurs, a third-line treatment is administered to the patient that can include a well-known third-line treatment to treat acute lymphoblastic leukemia. In some embodiments, the present invention provides a method of treating an acute lymphoblastic leukemia resistant to both first-line therapy and second-line therapy comprising administering X4P-001 as the third-line treatment. In some embodiments, the present invention provides a method of treating an acute lymphoblastic leukemia resistant to both first-line therapy and second-line therapy comprising administering X4P-001 in combination with another third-line treatment or standard of care third-line treatment for acute lymphoblastic leukaemia (e.g., radiotherapy, chemotherapy, immunotherapy, etc.).

In some embodiments, X4P-001 is administered as a sensitizer for the treatment of acute lymphoblastic leukemia. Without wishing to be bound by any particular theory, it is believed that X4P-001 increases the efficacy of the standard of care, first-line, second-line, or third-line treatments for acute lymphoblastic leukemia. In some embodiments, the present invention provides a method of treating an acute lymphoblastic leukemia in a patient in need thereof, comprising administering X4P-001 to the patient prior to administration of one or more of a standard of care, first-line, second-line, or third-line treatment. In some embodiments, administration of X4P-001 results in a more effective treatment of the acute lymphoblastic leukemia compared to treatment of acute lymphoblastic leukemia in the absence of administration of X4P-001. In some embodiments, the present invention provides a method of treating an acute lymphoblastic leukemia in a patient in need thereof, comprising administering X4P-001 to the patient after administration of one or more of a standard of care, first-line, second-line, or third-line treatment.

In some embodiments, the present invention provides a method of treating an acute lymphoblastic leukemia in a patient in need thereof, comprising administering X4P-001 to the patient in combination with an additional therapeutic agent suitable for treating the acute lymphoblastic leukemia. In some embodiments, the additional therapeutic agent is selected from methotrexate, nelarabine, asparaginase, inotuzumab ozogamicin, blinatumomab, daunorubicin HCl, cyclophosphamide, clorafabine, cytarabine, dasatinib, imatinib mesylate, ponatinib, ti sagenlecleucel, vincristine, mercaptopurine, pegaspargase, and prednisone. In some embodiments, the additional therapeutic agent is selected from inotuzumab ozogamicin (Besponsa®; Pfizer), imatinib mesylate, (Gleevec®; Novartis), blinatumomab (Blincyto®; Amgen), and Dasatinib (Sprycel®, BMS).

One of ordinary skill in the art will understand the amount and dosing regimen to administer such additional therapeutic agents for the treatment of acute lymphoblastic leukemia. By way of example, the administration of exemplary therapeutic agents suitable for treating acute lymphoblastic leukemia is summarized in Table 12, below.

TABLE 12
Exemplary Therapies for Acute lymphoblastic leukemia

| Therapeutic | |
|---|---|
| Agent | Dosing regimen |
| inotuzumab | 0.8 mg/m² on day 1, 0.5 mg/m² |
| ozogamicin | on day 8, and 0.5 mg/m² |
| (Besponsa ®; | on day 15 for a 21 day cycle. |
| Pfizer) | Pre-medicate with a corticosteroid, |
| | antipyretic, and antihistamine |
| | prior to all infusions. |
| imatinib mesylate | 600 mg once daily. |
| (Gleevec ®; | 400 mg once daily for mild to moderate |
| Novartis) | hepatic impairment and 300 mg once |
| | daily for severe hepatic impairment. |
| | Doses should be taken with a meal |
| | and a large glass of water. |
| | Dose can be dissolved in water or apple |
| | juice for patients having difficulty |
| | swallowing |
| blinatumomab | Hospitalization is recommended for the |
| (Blincyto ®; | first 9 days of the first cycle and the |
| Amgen) | first 2 days of the second cycle. |
| | A single cycle of treatment consists of |
| | 4 weeks ofc ontinuous intravenous infusion |
| | followed by a 2-week treatment-free interval. |
| | For patients at least 45 kg in weight, in Cycle 1, |
| | administer at 9 mcg/day on Days 1-7 and |
| | at 28 mcg/day on Days 8-28 or subsequent |
| | cycles, administer at 28 mcg/day on Days 1-28. |
| | Pre-medicate with dexamethasone 20 mg |
| | intravenously 1 hour prior to the first dose |
| | of each cycle, prior to a step dose (such as |
| | Cycle 1 day 8), or when restarting an infusion |
| | after an interruption of 4 or more hours. |
| | Administer as a continuous intravenous |
| | infusion at a constant flow rate using |
| | an infusion pump. The IV bag should |
| | be infused over 24 hours or 48 hours through |
| | a dedicated lumen |
| Dasatinib | 140 mg once daily administered |
| (Sprycel ®: | orally with or without a meal. |
| BMS) | |

### Chronic Lymphocytic Leukemia (CLL)

In some embodiments, the present invention provides a method of treating chronic lymphocytic leukemia in a patient in need thereof, comprising administering to the patient an effective amount of X4P-001 optionally in combination with one or more standard of care treatments, or a combination thereof, for chronic lymphocytic leukemia.

Standard of care treatments for chronic lymphocytic leukemia are well known to one of ordinary skill in the art and include radiotherapy, or chemotherapy, or a combination thereof. In some embodiments, the standard of care chemotherapy is selected from alemtuzumab, chlorambucil, ofatumumab, bendamustine, cyclophosphamide, fludarabine, obinutuzumab, ibrutinib, idelalisib, prednisone, rituximab, venetoclax, alkylating agents, and a combination of rituximab, cyclophosphamide, and dexamethasone. In some embodiments, the additional therapeutic agent is selected from venetoclax (Venclexta®; AbbVie), ibrutinib (Imbruvica®; Pharmacyclics/Janssen/AbbVie), obinutuzumab (Gazyva®; Genetech), and rituximab (Rituxan®; Biogen/Genetech).

In some embodiments, X4P-001 is administered to the patient as a monotherapy and as the first-line treatment for the chronic lymphocytic leukemia. In other embodiments, X4P-001 is administered to the patient as a first-line treatment in combination with a standard of care treatment for chronic lymphocytic leukemia (e.g., radiotherapy, or chemotherapy, or a combination thereof).

In some embodiments, when a standard of care treatment fails, such as when the chronic lymphocytic leukemia is partially resistant to a chemotherapy, a second-line treatment is used that can include a well-known second-line treatment to treat chronic lymphocytic leukemia. Accordingly, in some embodiments, the present invention provides a method of treating chronic lymphocytic leukemia in a patient wherein the cancer is resistant to a first-line therapy, said method comprising administering X4P-001 optionally in combination with a second-line treatment.

In some embodiments, the present invention provides a method of treating a resistant chronic lymphocytic leukemia comprising administering X4P-001 as the second-line treatment. In some embodiments, the present invention provides a method of treating a resistant chronic lymphocytic leukemia comprising administering X4P-001 in combination with another second-line treatment or standard of care second-line treatment for chronic lymphocytic leukemia (e.g., radiotherapy, chemotherapy, etc.). In some embodiments, the second-line treatment is selected from radiotherapy and chemotherapy.

In some instances when the first-line or second-line standard of care treatment fails, such as when chemotherapy continues to fail and remission occurs, a third-line treatment is administered to the patient that can include a well-known third-line treatment to treat chronic lymphocytic leukemia. In some embodiments, the present invention provides a method of treating a chronic lymphocytic leukemia resistant to both first-line therapy and second-line therapy comprising administering X4P-001 as the third-line treatment. In some embodiments, the present invention provides a method of treating a chronic lymphocytic leukemia resistant to both first-line therapy and second-line therapy comprising administering X4P-001 in combination with another third-line treatment or standard of care third-line treatment for chronic lymphocytic leukemia (e.g., radiotherapy, chemotherapy, etc.).

In some embodiments, X4P-001 is administered as a sensitizer for the treatment of chronic lymphocytic leukemia. Without wishing to be bound by any particular theory, it is believed that X4P-001 increases the efficacy of the standard of care, first-line, second-line, or third-line treatments for chronic lymphocytic leukemia. In some embodiments, the present invention provides a method of treating a chronic lymphocytic leukemia in a patient in need thereof, comprising administering X4P-001 to the patient prior to administration of one or more of a standard of care, first-line, second-line, or third-line treatment. In some embodiments, administration of X4P-001 results in a more effective treatment of the chronic lymphocytic leukemia compared to treatment of chronic lymphocytic leukemia in the absence of administration of X4P-001. In some embodiments, the present invention provides a method of treating a chronic lymphocytic leukemia in a patient in need thereof, comprising administering X4P-001 to the patient after administration of one or more of a standard of care, first-line, second-line, or third-line treatment.

In some embodiments, the present invention provides a method of treating a chronic lymphocytic leukemia in a patient in need thereof, comprising administering X4P-001 to the patient in combination with an additional therapeutic agent suitable for treating the chronic lymphocytic leukemia. In some embodiments, the additional therapeutic agent is selected from alemtuzumab, chlorambucil, ofatumumab, bendamustine, cyclophosphamide, fludarabine, obinutuzumab, ibrutinib, idelalisib, prednisone, rituximab, venetoclax, alkylating agents, and rituximab/cyclophosphamide/dexamethasone. In some embodiments, the additional therapeutic agent is selected from venetoclax (Venclexta®; AbbVie), ibrutinib (Imbruvica®; Pharmacyclics/Janssen/AbbVie), obinutuzumab (Gazyva®; Genetech) and rituximab (Rituxan®; Biogen/Genetech).

One of ordinary skill in the art will understand the amount and dosing regimen to administer such additional therapeutic agents for the treatment of chronic lymphocytic leukemia. By way of example, the administration of exemplary therapeutic agents suitable for treating chronic lymphocytic leukemia is summarized in Table 13, below.

TABLE 13
Exemplary Therapies for Chronic lymphocytic leukemia

| Therapeutic | |
|---|---|
| Agent | Dosing regimen |
| venetoclax | Initiate therapy at 20 mg once daily for 7 days, |
| (Venclexta ®; | followed by a weekly ramp-up dosing schedule to |
| AbbVie) | the recommended daily dose of 400 mg. |
| | Dose should be taken orally with a meal and water. |
| ibrutinib | 420 mg taken orally once daily until disease |
| (Imbruvica ®; | progression or unacceptable toxicity. |
| Pharmacyclics/ | Doses taken with a glass of water. |
| Janssen/AbbVie) | |
| obinutuzumab | 100 mg on day 1 and 900 mg on day 2 of Cycle 1. |
| (Gazyva ®; | 1000 mg on day 8 and 15 of Cycle 1. |
| Genetech) | 1000 mg on day 1 of Cycles 2-6. |
| | Pre-medicate for infusion reactions and |
| | tumor lysis syndrome. Dilute and |
| | administer as intravenous infusion. |
| | Do not administer as an intravenous |
| | push or bolus. |
| rituximab | 375 mg/m² in Cycle 1 and 500 mg/m² in |
| (Rituxan ®; | cycles 2-6. Methylprednisolone 100 mg IV |
| Biogen/ | or equivalent glucocorticoid is recommended |
| Genetech | 30 minutes prior to each infusion. Dilute |
| | and administer as intravenous infusion. Do |
| | not administer as an intravenous push or bolus. |

### Acute Myeloid Leukemia (AML)

In some embodiments, the present invention provides a method of treating acute myeloid leukemia in a patient in need thereof, comprising administering to the patient an effective amount of X4P-001 optionally in combination with one or more standard of care treatments, or a combination thereof, for acute myeloid leukemia.

Standard of care treatments for acute myeloid leukemia are well known to one of ordinary skill in the art and include radiotherapy, or chemotherapy, or a combination thereof. In some embodiments, the standard of care chemotherapy is selected from all-trans retinoic acid (ATRA)+arsenic trioxide, cyclophosphamide, cytarabine, and daunorubicin.

In some embodiments, X4P-001 is administered to the patient as a monotherapy and as the first-line treatment for the acute myeloid leukemia. In other embodiments, X4P-001 is administered to the patient as a first-line treatment in combination with a standard of care treatment for acute myeloid leukemia (e.g., radiotherapy, or chemotherapy, or a combination thereof).

In some embodiments, when a standard of care treatment fails, such as when the acute myeloid leukemia is partially resistant to a chemotherapy, a second-line treatment is used that can include a well-known second-line treatment to treat acute myeloid leukemia. Accordingly, in some embodiments, the present invention provides a method of treating acute myeloid leukemia in a patient wherein the cancer is resistant to a first-line therapy, said method comprising administering X4P-001 optionally in combination with a second-line treatment.

In some embodiments, the present invention provides a method of treating a resistant acute myeloid leukemia comprising administering X4P-001 as the second-line treatment. In some embodiments, the present invention provides a method of treating a resistant acute myeloid leukemia comprising administering X4P-001 in combination with another second-line treatment or standard of care second-line treatment for acute myeloid leukemia (e.g., radiotherapy, chemotherapy, allogeneic stem cell transplantation, immunotherapy, etc.). In some embodiments, the second-line treatment is selected from a chemotherapy in relapsed and refractory acute myeloid leukemia.

In some instances when the first-line or second-line standard of care treatment fails, such as when chemotherapy continues to fail and remission occurs, a third-line treatment is administered to the patient that can include a well-known third-line treatment to treat acute myeloid leukemia. In some embodiments, the present invention provides a method of treating an acute myeloid leukemia resistant to both first-line therapy and second-line therapy comprising administering X4P-001 as the third-line treatment. In some embodiments, the present invention provides a method of treating an acute myeloid leukemia resistant to both first-line therapy and second-line therapy comprising administering X4P-001 in combination with another third-line treatment or standard of care third-line treatment for acute myeloid leukemia (e.g., radiotherapy, chemotherapy, allogeneic stem cell transplantation, immunotherapy, etc.).

In some embodiments, X4P-001 is administered as a sensitizer for the treatment of acute myeloid leukemia. Without wishing to be bound by any particular theory, it is believed that X4P-001 increases the efficacy of the standard of care, first-line, second-line, or third-line treatments for acute myeloid leukemia. In some embodiments, the present invention provides a method of treating an acute myeloid leukemia in a patient in need thereof, comprising administering X4P-001 to the patient prior to administration of one or more of a standard of care, first-line, second-line, or third-line treatment. In some embodiments, administration of X4P-001 results in a more effective treatment of the acute myeloid leukemia compared to treatment of acute myeloid leukemia in the absence of administration of X4P-001. In some embodiments, the present invention provides a method of treating an acute myeloid leukemia in a patient in need thereof, comprising administering X4P-001 to the patient after administration of one or more of a standard of care, first-line, second-line, or third-line treatment.

In some embodiments, the present invention provides a method of treating an acute myeloid leukemia in a patient in need thereof, comprising administering X4P-001 to the patient in combination with an additional therapeutic agent suitable for treating the acute myeloid leukemia. In some embodiments, the additional therapeutic agent is selected from all-trans retinoic acid (ATRA)+arsenic trioxide, cyclophosphamide, cytarabine, and daunorubicin. One of ordinary skill in the art will understand the amount and dosing regimen to administer such additional therapeutic agents for the treatment of acute myeloid leukemia.

### Chronic Myeloid Leukemia (CML)

In some embodiments, the present invention provides a method of treating chronic myeloid leukemia in a patient in need thereof, comprising administering to the patient an effective amount of X4P-001 optionally in combination with one or more standard of care treatments, or a combination thereof, for chronic myeloid leukemia.

Standard of care treatments for chronic myeloid leukemia are well known to one of ordinary skill in the art and include radiotherapy, or chemotherapy, or a combination thereof. In some embodiments, the standard of care chemotherapy is selected from bosutinib, busulfan, cyclophosphide, cytarabine, dasatinib, imatinib, interferon, hydroxyurea, mechlorethamine HCl, nilotinib, omacetaxine mepesuccinate, and ponatinib. In some embodiments, the additional therapeutic agent is selected from dasatinib (Sprycel®; BMS), imatinib mesylate (Gleevec®; Novartis), and nilotinib (Tasigna®; Novartis).

In some embodiments, X4P-001 is administered to the patient as a monotherapy and as the first-line treatment for the chronic myeloid leukemia. In other embodiments, X4P-001 is administered to the patient as a first-line treatment in combination with a standard of care treatment for chronic myeloid leukemia (e.g., radiotherapy, or chemotherapy, or a combination thereof).

In some embodiments, when a standard of care treatment fails, such as when the chronic myeloid leukemia is partially resistant to a chemotherapy, a second-line treatment is used that can include a well-known second-line treatment to treat chronic myeloid leukemia. Accordingly, in some embodiments, the present invention provides a method of treating chronic myeloid leukemia in a patient wherein the cancer is resistant to a first-line therapy, said method comprising administering X4P-001 optionally in combination with a second-line treatment.

In some embodiments, the present invention provides a method of treating a resistant chronic myeloid leukemia comprising administering X4P-001 as the second-line treatment. In some embodiments, the present invention provides a method of treating a resistant chronic myeloid leukemia comprising administering X4P-001 in combination with another second-line treatment or standard of care second-line treatment for chronic myeloid leukemia (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat chronic myeloid leukemia. In some embodiments, the present invention provides a method of treating a chronic myeloid leukemia resistant to both first-line therapy and second-line therapy comprising administering X4P-001 as the third-line treatment. In some embodiments, the present invention provides a method of treating a chronic myeloid leukemia resistant to both first-line therapy and second-line therapy comprising administering X4P-001 in combination with another third-line treatment or standard of care third-line treatment for chronic myeloid leukemia (e.g., radiotherapy, chemotherapy, etc.).

In some embodiments, X4P-001 is administered as a sensitizer for the treatment of chronic myeloid leukemia. Without wishing to be bound by any particular theory, it is believed that X4P-001 increases the efficacy of the standard of care, first-line, second-line, or third-line treatments for chronic myeloid leukemia. In some embodiments, the present invention provides a method of treating a chronic myeloid leukemia in a patient in need thereof, comprising administering X4P-001 to the patient prior to administration of one or more of a standard of care, first-line, second-line, or third-line treatment. In some embodiments, administration of X4P-001 results in a more effective treatment of the chronic myeloid leukemia compared to treatment of chronic myeloid leukemia in the absence of administration of X4P-001. In some embodiments, the present invention provides a method of treating a chronic myeloid leukemia in a patient in need thereof, comprising administering X4P-001 to the patient after administration of one or more of a standard of care, first-line, second-line, or third-line treatment.

In some embodiments, the present invention provides a method of treating a chronic myeloid leukemia in a patient in need thereof, comprising administering X4P-001 to the patient in combination with an additional therapeutic agent suitable for treating the chronic myeloid leukemia. In some embodiments, the additional therapeutic agent is selected from bosutinib, busulfan, cyclophosphide, cytarabine, dasatinib, imatinib, interferon, hydroxyurea, mechlorethamine HCl, nilotinib, omacetaxine mepesuccinate, and ponatinib. In some embodiments, the additional therapeutic agent is selected from dasatinib (Sprycel®; BMS), imatinib mesylate (Gleevec®; Novartis), and nilotinib (Tasigna®; Novartis).

One of ordinary skill in the art will understand the amount and dosing regimen to administer such additional therapeutic agents for the treatment of chronic myeloid leukemia. By way of example, the administration of exemplary therapeutic agents suitable for treating chronic myeloid leukemia is summarized in Table 14, below.

TABLE 14
Exemplary Therapies for Chronic Myeloid Leukemia

| TherapeuticAgent | Dosing regimen |
|---|---|
| dasatinib | 100 mg once daily for chronic phase CML. |
| (Sprycel ®; BMS) | 140 mg once daily for accelerated phase, myeloid, or |
| | lymphoid blast phase CML. |
| | Administered orally, with or without a meal. |
| imatinib mesylate | Recommended dose is oral 400 mg/day for adult patients in |
| (Gleevec ®; Novartis) | chronic phase (CP) CML and 600 mg/day for adult patients |
| | in accelerated phase or blast crisis. |
| | A dose increase from 400 mg to 600 mg in adult patients |
| | with chronic phase disease, or from 600 mg to 800 mg |
| | (given as 400 mg twice daily) in adult patients in accelerated |
| | phase or blast crisis may be considered in the absence of |
| | severe adverse drug reaction and severe non-leukemia |
| | related neutropenia or thrombocytopenia in the following |
| | circumstances: disease progression (at any time), failure to |
| | achieve a satisfactory hematologic response after at least 3 |
| | months of treatment, failure to achieve a cytogenetic |
| | response after 6-12 months of treatment, or loss of a |
| | previously achieved hematologic or cytogenetic response. |
| | Recommended dose for children with newly diagnosed |
| | Philadelphia chromosome positive (Ph+) CML is 340 |
| | mg/m²/day (not to exceed 600 mg). |
| | The recommended dose is 260 mg/m²/day for children with |
| | Ph+ chronic phase CML recurrent after stem cell transplant |
| | or who are resistant to interferon-alpha therapy. |
| nilotinib | 300 mg orally b.i.d. for newly diagnosed Philadelphia |
| (Tasigna ®; Novartis) | chromosome positive (Ph+) chronic phase CML. |
| | 400 mg orally b.i.d. for resistant or intolerant Ph+ chronic |
| | phase CML and accelerated phase CML. |
| | Administer approximately 12 hours apart and must not take |
| | with food. |
| | Swallow the capsules whole with water. |
| | Do not consume food for at least 2 hours before the dose is |
| | taken and for at least one hour after. |
| | Dose adjustment may be required for hematologic and non- |
| | hematologic toxicities, and drug interactions. |
| | A lower starting dose is recommended in patients with |
| | hepatic impairment (at baseline). |

### Multiple Myeloma

In some embodiments, the present invention provides a method of treating multiple myeloma in a patient in need thereof, comprising administering to the patient an effective amount of X4P-001 optionally in combination with one or more standard of care treatments, or a combination thereof, for multiple myeloma.

Standard of care treatments for multiple myeloma are well known to one of ordinary skill in the art and include chemotherapy, autologous hemopoietic stem-cell transplantation (ASCT), or a combination thereof. In some embodiments, the standard of care chemotherapy is selected from bortezomib, carfilzomib, carmustine, cyclophosphamide, daratumumab, doxorubicin HCl liposome, elotuzumab, ixazomib citrate, lenalidomide; melphalan, pamidronate disodium, panobinostat, plerixafor, pomalidomide, thalidomide, and zoledronic acid. In some embodiments, the additional therapeutic agent is elotuzumab (Empliciti®; BMS).

In some embodiments, X4P-001 is administered to the patient as a monotherapy and as the first-line treatment for the multiple myeloma. In other embodiments, X4P-001 is administered to the patient as a first-line treatment in combination with a standard of care treatment for multiple myeloma (e.g., chemotherapy, autologous hemopoietic stem-cell transplantation (ASCT), or a combination thereof).

In some embodiments, when a standard of care treatment fails, such as when the multiple myeloma is partially resistant to a chemotherapy, a second-line treatment is used that can include a well-known second-line treatment to treat multiple myeloma. Accordingly, in some embodiments, the present invention provides a method of treating multiple myeloma in a patient wherein the cancer is resistant to a first-line therapy, said method comprising administering X4P-001 optionally in combination with a second-line treatment.

In some embodiments, the present invention provides a method of treating a resistant multiple myeloma comprising administering X4P-001 as the second-line treatment. In some embodiments, the present invention provides a method of treating a resistant multiple myeloma comprising administering X4P-001 in combination with another second-line treatment or standard of care second-line treatment for multiple myeloma (e.g., chemotherapy, immunotherapy, etc.). In some embodiments, the second-line treatment is selected from a chemotherapy in relapsed and refractory multiple myeloma.

In some instances when the first-line or second-line standard of care treatment fails, such as when chemotherapy continues to fail and remission occurs, a third-line treatment is administered to the patient that can include a well-known third-line treatment to treat multiple myeloma. In some embodiments, the present invention provides a method of treating a multiple myeloma resistant to both first-line therapy and second-line therapy comprising administering X4P-001 as the third-line treatment. In some embodiments, the present invention provides a method of treating a multiple myeloma resistant to both first-line therapy and second-line therapy comprising administering X4P-001 in combination with another third-line treatment or standard of care third-line treatment for multiple myeloma (e.g., chemotherapy, immunotherapy, etc.).

In some embodiments, X4P-001 is administered as a sensitizer for the treatment of multiple myeloma. Without wishing to be bound by any particular theory, it is believed that X4P-001 increases the efficacy of the standard of care, first-line, second-line, or third-line treatments for multiple myeloma. In some embodiments, the present invention provides a method of treating a multiple myeloma in a patient in need thereof, comprising administering X4P-001 to the patient prior to administration of one or more of a standard of care, first-line, second-line, or third-line treatment. In some embodiments, administration of X4P-001 results in a more effective treatment of the multiple myeloma compared to treatment of multiple myeloma in the absence of administration of X4P-001. In some embodiments, the present invention provides a method of treating a multiple myeloma in a patient in need thereof, comprising administering X4P-001 to the patient after administration of one or more of a standard of care, first-line, second-line, or third-line treatment.

In some embodiments, the present invention provides a method of treating a multiple myeloma in a patient in need thereof, comprising administering X4P-001 to the patient in combination with an additional therapeutic agent suitable for treating the multiple myeloma. In some embodiments, the additional therapeutic agent is selected from bortezomib, carfilzomib, carmustine, cyclophosphamide, daratumumab, doxorubicin HCl liposome, elotuzumab, ixazomib citrate, lenalidomide; melphalan, pamidronate disodium, panobinostat, plerixafor, pomalidomide, thalidomide, and zoledronic acid. In some embodiments, the additional therapeutic agent is elotuzumab (Empliciti®; BMS).

One of ordinary skill in the art will understand the amount and dosing regimen to administer such additional therapeutic agents for the treatment of multiple myeloma. By way of example, the administration of exemplary therapeutic agents suitable for treating multiple myeloma is summarized in Table15, below.

TABLE 15
Exemplary Therapies for Multiple Myeloma

| Therapeutic Agent | Dosing regimen |
|---|---|
| elotuzumab | 10 mg/kg administered intravenously every week for the first two |
| (Empliciti ® BMS). | cycles and every 2 weeks thereafter until disease progression or |
| | unacceptable toxicity. |
| | Pre-medicate with dexamethasone, diphenhydramine, ranitidine |
| | and acetaminophen |

### Colorectal Cancer

In some embodiments, the present invention provides a method of treating colorectal cancer in a patient in need thereof, comprising administering to the patient an effective amount of X4P-001 optionally in combination with one or more standard of care treatments, or a combination thereof, for colorectal cancer.

Standard of care treatments for colorectal cancer are well known to one of ordinary skill in the art and include surgery, radiotherapy, chemotherapy, or targeted immunotherapy, or a combination thereof. In some embodiments, the standard of care chemotherapy is selected from bevacizumab, capecitabine, camptothecin-11 (Camptosar®; Pfizer), cetuximab, 5-fluorouracil injection, irinotecan HCl, leucovorin calcium, nivolumab, oxaliplatin, panitumumab, pembrolizumab, ramucirumab, regorafenib, trifluridine+tipiracil HCl (TAS102), and Ziv-Aflibercept. In some embodiments, the additional therapeutic agent is selected from bevacizumab (Avastin®; Genentech/Roche), panitumumab (Vectibix®; Amgen), pembrolizumab (Keytruda®; Merck), oxaliplatin (Eloxatin; Sanofi-Aventis), and capecitabine (Xeloda®; Hoffmann-La Roche).

In some embodiments, X4P-001 is administered to the patient as a monotherapy and as the first-line treatment for the colorectal cancer. In other embodiments, X4P-001 is administered to the patient as a first-line treatment in combination with a standard of care treatment for colorectal cancer (e.g., surgery, radiotherapy, chemotherapy, or targeted immunotherapy, or a combination thereof).

In some embodiments, when a standard of care treatment fails, such as when surgery fails to remove all cancerous tissue or the colorectal cancer is partially resistant to a chemotherapy, a second-line treatment is used that can include a well-known second-line treatment to treat colorectal cancer. Accordingly, in some embodiments, the present invention provides a method of treating colorectal cancer in a patient wherein the cancer is resistant to a first-line therapy, said method comprising administering X4P-001 optionally in combination with a second-line treatment.

In some embodiments, the present invention provides a method of treating a resistant colorectal cancer comprising administering X4P-001 as the second-line treatment. In some embodiments, the present invention provides a method of treating a resistant colorectal cancer comprising administering X4P-001 in combination with another second-line treatment or standard of care second-line treatment for colorectal cancer (e.g., radiotherapy, chemotherapy, targeted immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat colorectal cancer. In some embodiments, the present invention provides a method of treating a colorectal cancer resistant to both first-line therapy and second-line therapy comprising administering X4P-001 as the third-line treatment. In some embodiments, the present invention provides a method of treating a colorectal cancer resistant to both first-line therapy and second-line therapy comprising administering X4P-001 in combination with another third-line treatment or standard of care third-line treatment for colorectal cancer (e.g., radiotherapy, chemotherapy, targeted immunotherapy, etc.).

In some embodiments, X4P-001 is administered as a sensitizer for the treatment of colorectal cancer. Without wishing to be bound by any particular theory, it is believed that X4P-001 increases the efficacy of the standard of care, first-line, second-line, or third-line treatments for colorectal cancer. In some embodiments, the present invention provides a method of treating a colorectal cancer in a patient in need thereof, comprising administering X4P-001 to the patient prior to administration of one or more of a standard of care, first-line, second-line, or third-line treatment. In some embodiments, administration of X4P-001 results in a more effective treatment of the colorectal cancer compared to treatment of colorectal cancer in the absence of administration of X4P-001. In some embodiments, the present invention provides a method of treating a colorectal cancer in a patient in need thereof, comprising administering X4P-001 to the patient after administration of one or more of a standard of care, first-line, second-line, or third-line treatment.

In some embodiments, the present invention provides a method of treating a colorectal cancer in a patient in need thereof, comprising administering X4P-001 to the patient in combination with an additional therapeutic agent suitable for treating the colorectal cancer. In some embodiments, the additional therapeutic agent is selected from bevacizumab, capecitabine, camptothecin-11 (Camptosar®; Pfizer), cetuximab, 5-fluorouracil injection, irinotecan HCl, leucovorin calcium, nivolumab, oxaliplatin, panitumumab, pembrolizumab, ramucirumab, regorafenib, trifluridine+tipiracil HCl (TAS102), and Ziv-Aflibercept. In some embodiments, the additional therapeutic agent is selected from bevacizumab (Avastin®; Genentech/Roche), panitumumab (Vectibix®; Amgen), pembrolizumab (Keytruda®; Merck), oxaliplatin (Eloxatin; Sanofi-Aventis), and capecitabine (Xeloda®; Hoffmann-La Roche).

One of ordinary skill in the art will understand the amount and dosing regimen to administer such additional therapeutic agents for the treatment of colorectal cancer. By way of example, the administration of exemplary therapeutic agents suitable for treating colorectal cancer is summarized in Table 16, below.

TABLE 16
Exemplary Therapies for Colorectal Cancer

| Therapeutic Agent | Dosing regimen |
|---|---|
| bevacizumab | 5-15 mg/kg IV every 2 weeks |
| (Avastin ®; Genentech/Roche) | Do not administer as an IV push or bolus. |
| | Do not initiate for 28 days following major surgery and until |
| | surgical wound is fully healed. |
| panitumumab | Administer at 6 mg/kg every 14 days as an intravenous |
| (Vectibix ®; Amgen) | infusion over 60 minutes (≤1000 mg) or 90 minutes (>1000 mg). |
| | Reduce infusion rate by 50% for mild reactions; immediately |
| | and permanently discontinue for severe reactions. |
| | Withhold for severe or intolerable dermatological toxicity; may |
| | resume at 50% of dose if toxicity improves. |
| pembrolizumab | 200 mg every 3 weeks. |
| (Keytruda ®; Merck) | Administer as an intravenous infusion over 30 minutes. |
| oxaliplatin | Administer in combination with 5-fluorouracil/leucovorin |
| (Eloxatin; Sanofi-Aventis) | every 2 weeks. |
| | Day 1: 85 mg/m² intravenous infusion in 250-500 mL 5% |
| | dextrose Injection, USP and leucovorin 200 mg/m² intravenous |
| | infusion in 5% dextrose Injection, USP both given over 120 |
| | minutes at the same time in separate bags using a Y-line, |
| | followed by 5-fluorouracil 400 mg/m² intravenous bolus given |
| | over 2-4 minutes, followed by 5-fluorouracil 600 mg/m² |
| | intravenous infusion in 500 mL 5% dextrose Injection, USP |
| | (recommended) as a 22-hour continuous infusion. |
| | Day 2: leucovorin 200 mg/m² intravenous infusion over 120 |
| | minutes, followed by 5-fluorouracil 400 mg/m² IV bolus given |
| | over 2-4 minutes, followed by 5-fluorouracil 600 mg/m² |
| | intravenous infusion in 500 mL 5% dextrose Injection, USP |
| | (recommended) as a 22-hour continuous infusion. |
| | Reduce the dose to 75 mg/m² (adjuvant setting) or 65 mg/m² |
| | (advanced colorectal cancer) if there are persistent grade 2 |
| | neurosensory events that do not resolve. |
| | After recovery from grade 3/4 gastrointestinal toxicities |
| | (despite prophylactic treatment) or grade 4 neutropenia or |
| | grade 3/4 thrombocytopenia, delay next dose until neutrophils |
| | ≥1.5 × 109/L and platelets ≥75 × 109/L. |
| | Discontinue if there are persistent Grade 3 neurosensory events. |
| | Never reconstitute or prepare final dilution with a sodium |
| | chloride solution or other chloride-containing solutions |
| capecitabine | 1250 mg/m² b.i.d. orally for 2 weeks followed by a one week |
| (Xeloda ®; Hoffmann- | rest period in 3-week cycles for monotherapy. |
| LaRoche) | Adjuvant treatment is recommended for a total of 6 months |
| | (8 cycles). |
| | 1250 mg/m² twice daily for 2 weeks followed by a 7-day rest |
| | period, combined with docetaxel at 75 mg/m² as a 1-hour IV |
| | infusion every 3 weeks. |
| | Dosage may need to be individualized to optimize patient |
| | management. |
| | Reduce the dose by 25% in patients with moderate renal |
| | impairment. |
| | Take with water within 30 min after a meal |

### Gall Bladder Cancer, Biliary Tract Cancer, and Gastrointestinal Stromal Tumors (GIST)

In some embodiments, the present invention provides a method of treating gall bladder cancer, biliary tract cancer, or gastrointestinal stromal tumors in a patient in need thereof, comprising administering to the patient an effective amount of X4P-001 optionally in combination with one or more standard of care treatments, or a combination thereof, for gall bladder cancer, biliary tract cancer, or gastrointestinal stromal tumors.

Standard of care treatments for gall bladder cancer, biliary tract cancer, or gastrointestinal stromal tumors are well known to one of ordinary skill in the art and include surgery, radiotherapy, or chemotherapy, or a combination thereof. In some embodiments, the standard of care chemotherapy for gall bladder cancer, biliary tract cancer, or gastrointestinal stromal tumors is selected from gemcitabine, fluoropyrimidines, platinum agents, docetaxel, erlotinib, imatinib mesylate; regorafenib; sunitinib malate, docetaxel, doxorubicin HCl, 5-fluorouracil injection, mitomycin C, ramucirumab, and trastuzumab. In some embodiments, the additional therapeutic agent is selected from imatinib mesylate (Gleevec®; Novartis), sunitinib (Sutent®; Pfizer), and ramucirumab (Cyramza®; Lilly).

In some embodiments, X4P-001 is administered to the patient as a monotherapy and as the first-line treatment for the gall bladder cancer, biliary tract cancer, or gastrointestinal stromal tumors. In other embodiments, X4P-001 is administered to the patient as a first-line treatment in combination with a standard of care treatment for gall bladder cancer, biliary tract cancer, or gastrointestinal stromal tumors (e.g., radiotherapy, or chemotherapy, or a combination thereof).

In some embodiments, when a standard of care treatment fails, such as when surgery fails to remove all cancerous tissue or the gall bladder cancer, biliary tract cancer, or gastrointestinal stromal tumors is partially resistant to a chemotherapy, a second-line treatment is used that can include a well-known second-line treatment to treat gall bladder cancer, biliary tract cancer, or gastrointestinal stromal tumors. Accordingly, in some embodiments, the present invention provides a method of treating gall bladder cancer, biliary tract cancer, or gastrointestinal stromal tumors in a patient wherein the cancer is resistant to a first-line therapy, said method comprising administering X4P-001 optionally in combination with a second-line treatment.

In some embodiments, the present invention provides a method of treating a resistant gall bladder cancer, biliary tract cancer, or gastrointestinal stromal tumors comprising administering X4P-001 as the second-line treatment. In some embodiments, the present invention provides a method of treating a resistant gall bladder cancer, biliary tract cancer, or gastrointestinal stromal tumors comprising administering X4P-001 in combination with another second-line treatment or standard of care second-line treatment for gall bladder cancer, biliary tract cancer, or gastrointestinal stromal tumors (e.g., radiotherapy, chemotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat gall bladder cancer, biliary tract cancer, or gastrointestinal stromal tumors. In some embodiments, the present invention provides a method of treating a gall bladder cancer, biliary tract cancer, or gastrointestinal stromal tumors resistant to both first-line therapy and second-line therapy comprising administering X4P-001 as the third-line treatment. In some embodiments, the present invention provides a method of treating a gall bladder cancer, biliary tract cancer, or gastrointestinal stromal tumors resistant to both first-line therapy and second-line therapy comprising administering X4P-001 in combination with another third-line treatment or standard of care third-line treatment for gall bladder cancer, biliary tract cancer, or gastrointestinal stromal tumors (e.g., radiotherapy, chemotherapy, etc.).

In some embodiments, X4P-001 is administered as a sensitizer for the treatment of gall bladder cancer, biliary tract cancer, or gastrointestinal stromal tumors. Without wishing to be bound by any particular theory, it is believed that X4P-001 increases the efficacy of the standard of care, first-line, second-line, or third-line treatments for gall bladder cancer, biliary tract cancer, or gastrointestinal stromal tumors. In some embodiments, the present invention provides a method of treating a gall bladder cancer, biliary tract cancer, or gastrointestinal stromal tumors in a patient in need thereof, comprising administering X4P-001 to the patient prior to administration of one or more of a standard of care, first-line, second-line, or third-line treatment. In some embodiments, administration of X4P-001 results in a more effective treatment of the gall bladder cancer, biliary tract cancer, or gastrointestinal stromal tumors compared to treatment of gall bladder cancer, biliary tract cancer, or gastrointestinal stromal tumors in the absence of administration of X4P-001. In some embodiments, the present invention provides a method of treating a gall bladder cancer, biliary tract cancer, or gastrointestinal stromal tumors in a patient in need thereof, comprising administering X4P-001 to the patient after administration of one or more of a standard of care, first-line, second-line, or third-line treatment.

In some embodiments, the present invention provides a method of treating a gall bladder cancer, biliary tract cancer, or gastrointestinal stromal tumors in a patient in need thereof, comprising administering X4P-001 to the patient in combination with an additional therapeutic agent suitable for treating the gall bladder cancer, biliary tract cancer, or gastrointestinal stromal tumors. In some embodiments, the additional therapeutic agent is selected from gemcitabine, fluoropyrimidines, platinum agents, docetaxel, erlotinib, imatinib mesylate; regorafenib; sunitinib malate, docetaxel, doxorubicin HCl, 5-fluorouracil injection, mitomycin C, ramucirumab, and trastuzumab. In some embodiments, the additional therapeutic agent is selected from imatinib mesylate (Gleevec®; Novartis), sunitinib (Sutent®; Pfizer), and ramucirumab (Cyramza®; Lilly).

One of ordinary skill in the art will understand the amount and dosing regimen to administer such additional therapeutic agents for the treatment of gall bladder cancer, biliary tract cancer, or gastrointestinal stromal tumors. By way of example, the administration of exemplary therapeutic agents suitable for treating gall bladder cancer, biliary tract cancer, or gastrointestinal stromal tumors is summarized in Table 17, below.

TABLE 17
Exemplary Therapies for Gall Bladder Cancer, Biliary Tract Cancer, or
Gastrointestinal Stromal Tumors

| Therapeutic Agent | Dosing regimen |
|---|---|
| imatinib mesylate | 400 mg/day for adult patients. |
| (Gleevee ®; Novartis) | A dose increase up to 800 mg daily (given as 400 mg twice daily) |
| | may be considered, as clinically indicated, in patients showing |
| | clear signs or symptoms of disease progression at a lower dose |
| | and in the absence of severe adverse drug reactions |
| sunitinib | 50 mg orally once daily, with or without food, 4 weeks on |
| (Sutent ®; Pfizer) | treatment followed by 2 weeks off. |
| | Dose interruptions and/or dose adjustments of 12.5 mg |
| | recommended based on individual safety and tolerability. |
| ramucirumab | 8 mg/kg every 2 weeks as a single agent or in combination with |
| (Cyramza ®; Lilly) | weekly paclitaxel. |
| | Intravenous infusion only. |
| | Do not administer as an intravenous push or bolus. |

### Hodgkin's Lymphoma

In some embodiments, the present invention provides a method of treating Hodgkin's lymphoma in a patient in need thereof, comprising administering to the patient an effective amount of X4P-001 optionally in combination with one or more standard of care treatments, or a combination thereof, for Hodgkin's lymphoma.

Standard of care treatments for Hodgkin's lymphoma are well known to one of ordinary skill in the art and include radiotherapy, chemotherapy, or a combination thereof. In some embodiments, the standard of care chemotherapy is selected from bleomycin, brentuximab vedotin, carmustine, chlorambucil, cyclophosphamide, dacarbazine, doxorubicin HCl, ibrutinib, lomustine, mechlorethamine HCl, nivolumab, pembrolizumab, prednisone, procarbazine HCl, vinblastin sulfate, and vincristine sulfate. In some embodiments, the additional therapeutic agent is ibrutinib (Imbruvica®; Pharmacyclics/Janssen/AbbVie).

In some embodiments, X4P-001 is administered to the patient as a monotherapy and as the first-line treatment for the Hodgkin's lymphoma. In other embodiments, X4P-001 is administered to the patient as a first-line treatment in combination with a standard of care treatment for Hodgkin's lymphoma (e.g., radiotherapy, chemotherapy, or a combination thereof).

In some embodiments, when a standard of care treatment fails, such as when the Hodgkin's lymphoma is partially resistant to a chemotherapy, a second-line treatment is used that can include a well-known second-line treatment to treat Hodgkin's lymphoma. Accordingly, in some embodiments, the present invention provides a method of treating Hodgkin's lymphoma in a patient wherein the cancer is resistant to a first-line therapy, said method comprising administering X4P-001 optionally in combination with a second-line treatment.

In some embodiments, the present invention provides a method of treating a resistant Hodgkin's lymphoma comprising administering X4P-001 as the second-line treatment. In some embodiments, the present invention provides a method of treating a resistant Hodgkin's lymphoma comprising administering X4P-001 in combination with another second-line treatment or standard of care second-line treatment for Hodgkin's lymphoma (e.g., radiotherapy, chemotherapy, immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat Hodgkin's lymphoma. In some embodiments, the present invention provides a method of treating a Hodgkin's lymphoma resistant to both first-line therapy and second-line therapy comprising administering X4P-001 as the third-line treatment. In some embodiments, the present invention provides a method of treating a Hodgkin's lymphoma resistant to both first-line therapy and second-line therapy comprising administering X4P-001 in combination with another third-line treatment or standard of care third-line treatment for Hodgkin's lymphoma (e.g., radiotherapy, chemotherapy, immunotherapy, etc.).

In some embodiments, X4P-001 is administered as a sensitizer for the treatment of Hodgkin's lymphoma. Without wishing to be bound by any particular theory, it is believed that X4P-001 increases the efficacy of the standard of care, first-line, second-line, or third-line treatments for Hodgkin's lymphoma. In some embodiments, the present invention provides a method of treating a Hodgkin's lymphoma in a patient in need thereof, comprising administering X4P-001 to the patient prior to administration of one or more of a standard of care, first-line, second-line, or third-line treatment. In some embodiments, administration of X4P-001 results in a more effective treatment of the Hodgkin's lymphoma compared to treatment of Hodgkin's lymphoma in the absence of administration of X4P-001. In some embodiments, the present invention provides a method of treating a Hodgkin's lymphoma in a patient in need thereof, comprising administering X4P-001 to the patient after administration of one or more of a standard of care, first-line, second-line, or third-line treatment.

In some embodiments, the present invention provides a method of treating a Hodgkin's lymphoma in a patient in need thereof, comprising administering X4P-001 to the patient in combination with an additional therapeutic agent suitable for treating the Hodgkin's lymphoma. In some embodiments, the additional therapeutic agent is selected from bleomycin, brentuximab vedotin, carmustine, chlorambucil, cyclophosphamide, dacarbazine, doxorubicin HCl, ibrutinib, lomustine, mechlorethamine HCl, nivolumab, pembrolizumab, prednisone, procarbazine HCl, vinblastin sulfate, and vincristine sulfate. In some embodiments, the additional therapeutic agent is ibrutinib (Imbruvica®; Pharmacyclics/Janssen/AbbVie).

One of ordinary skill in the art will understand the amount and dosing regimen to administer such additional therapeutic agents for the treatment of Hodgkin's lymphoma. By way of example, the administration of exemplary therapeutic agents suitable for treating Hodgkin's lymphoma is summarized in Table 18, below.

TABLE 18
Exemplary Therapies for Hodgkin's Lymphoma

| Therapeutic Agent | Dosing regimen |
|---|---|
| ibrutinib | 420-560 mg taken orally once daily until disease progression |
| (Imbruvica ®; | or unacceptable toxicity. |
| Pharmacyclics/Janssen/AbbVie) | Doses taken with a glass of water. |

### Non-Hodgkin's Lymphoma

In some embodiments, the present invention provides a method of treating non-Hodgkin's lymphoma in a patient in need thereof, comprising administering to the patient an effective amount of X4P-001 optionally in combination with one or more standard of care treatments, or a combination thereof, for non-Hodgkin's lymphoma.

Standard of care treatments for non-Hodgkin's lymphoma are well known to one of ordinary skill in the art and include chemotherapy, or stem cell transplantation, or a combination thereof. In some embodiments, the standard of care chemotherapy is selected from acalabrutinib, axicabtagene ciloleucel, belinostat, bendamustine HCl, bleomycin, bortezomib, brentuximab vedotin, carmustine, chlorambucil, copanlisib HCl, cyclophosphamide, cytarabine liposome, denileukin diftitox, dexamethasone, doxorubicin HCl, ibritumomab tiuxetan, ibrutinib, idelalisib, lenalidomide, mechlorethamine HCl, methotrexate, nelarabine, obinutuzumab, plerixafor, pralatrexate, prednisone, rec-interferon-alfa-2b, rituximab, rituximab+hyaluronidase, romidepsin, vinblastine sulfate, vincristine sulfate, and vorinostat. In some embodiments, the additional therapeutic agent is ibrutinib (Imbruvica®; Pharmacyclics/Janssen/AbbVie).

In some embodiments, X4P-001 is administered to the patient as a monotherapy and as the first-line treatment for the non-Hodgkin's lymphoma. In other embodiments, X4P-001 is administered to the patient as a first-line treatment in combination with a standard of care treatment for non-Hodgkin's lymphoma (e.g., chemotherapy, stem cell transplantation, or a combination thereof).

In some embodiments, when a standard of care treatment fails, such as when the non-Hodgkin's lymphoma is partially resistant to a chemotherapy, a second-line treatment is used that can include a well-known second-line treatment to treat non-Hodgkin's lymphoma. Accordingly, in some embodiments, the present invention provides a method of treating non-Hodgkin's lymphoma in a patient wherein the cancer is resistant to a first-line therapy, said method comprising administering X4P-001 optionally in combination with a second-line treatment.

In some embodiments, the present invention provides a method of treating a resistant non-Hodgkin's lymphoma comprising administering X4P-001 as the second-line treatment. In some embodiments, the present invention provides a method of treating a resistant non-Hodgkin's lymphoma comprising administering X4P-001 in combination with another second-line treatment or standard of care second-line treatment for non-Hodgkin's lymphoma (e.g., chemotherapy, immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat non-Hodgkin's lymphoma. In some embodiments, the present invention provides a method of treating a non-Hodgkin's lymphoma resistant to both first-line therapy and second-line therapy comprising administering X4P-001 as the third-line treatment. In some embodiments, the present invention provides a method of treating a non-Hodgkin's lymphoma resistant to both first-line therapy and second-line therapy comprising administering X4P-001 in combination with another third-line treatment or standard of care third-line treatment for non-Hodgkin's lymphoma (e.g., chemotherapy, immunotherapy, etc.).

In some embodiments, X4P-001 is administered as a sensitizer for the treatment of non-Hodgkin's lymphoma. Without wishing to be bound by any particular theory, it is believed that X4P-001 increases the efficacy of the standard of care, first-line, second-line, or third-line treatments for non-Hodgkin's lymphoma. In some embodiments, the present invention provides a method of treating a non-Hodgkin's lymphoma in a patient in need thereof, comprising administering X4P-001 to the patient prior to administration of one or more of a standard of care, first-line, second-line, or third-line treatment. In some embodiments, administration of X4P-001 results in a more effective treatment of the non-Hodgkin's lymphoma compared to treatment of non-Hodgkin's lymphoma in the absence of administration of X4P-001. In some embodiments, the present invention provides a method of treating a non-Hodgkin's lymphoma in a patient in need thereof, comprising administering X4P-001 to the patient after administration of one or more of a standard of care, first-line, second-line, or third-line treatment.

In some embodiments, the present invention provides a method of treating a non-Hodgkin's lymphoma in a patient in need thereof, comprising administering X4P-001 to the patient in combination with an additional therapeutic agent suitable for treating the non-Hodgkin's lymphoma. In some embodiments, the additional therapeutic agent is selected from acalabrutinib, axicabtagene ciloleucel, belinostat, bendamustine HCl, bleomycin, bortezomib, brentuximab vedotin, carmustine, chlorambucil, copanlisib HCl, cyclophosphamide, cytarabine liposome, denileukin diftitox, dexamethasone, doxorubicin HCl, ibritumomab tiuxetan, ibrutinib, idelalisib, lenalidomide, mechlorethamine HCl, methotrexate, nelarabine, obinutuzumab, plerixafor, pralatrexate, prednisone, rec-interferon-alfa-2b, rituximab, rituximab+hyaluronidase, romidepsin, vinblastine sulfate, vincristine sulfate, and vorinostat. In some embodiments, the additional therapeutic agent is ibrutinib (Imbruvica®; Pharmacyclics/Janssen/AbbVie).

One of ordinary skill in the art will understand the amount and dosing regimen to administer such additional therapeutic agents for the treatment of non-Hodgkin's lymphoma. By way of example, the administration of exemplary therapeutic agents suitable for treating non-Hodgkin's lymphoma is summarized in Table 19, below.

TABLE 19
Exemplary Therapies for Non-Hodgkin's Lymphoma

| Therapeutic Agent | Dosing regimen |
|---|---|
| ibrutinib | 420-560 mg taken orally once daily until disease |
| (Imbruvica ®; | progression or unacceptable toxicity. |
| Pharmacyclics/Janssen/AbbVie) | Doses taken with a glass of water. |

### Mantle Cell Lymphoma

In some embodiments, the present invention provides a method of treating mantle cell lymphoma in a patient in need thereof, comprising administering to the patient an effective amount of X4P-001 optionally in combination with one or more standard of care treatments, or a combination thereof, for mantle cell lymphoma.

Standard of care treatments for mantle cell lymphoma are well known to one of ordinary skill in the art and include radiotherapy, or chemotherapy, or a combination thereof. In some embodiments, the standard of care chemotherapy is selected from ibrutinib, bortezornib, and acalabrutinib. In some embodiments, the additional therapeutic agent is selected from acalabrutinib (Calquence®; AstraZeneca), bortezomib (Velcade®; Takeda), and ibrutinib (Imbruvica®; Pharmacyclics/Janssen/AbbVie).

In some embodiments, X4P-001 is administered to the patient as a monotherapy and as the first-line treatment for the mantle cell lymphoma. In other embodiments, X4P-001 is administered to the patient as a first-line treatment in combination with a standard of care treatment for mantle cell lymphoma (e.g., radiotherapy, or chemotherapy, or a combination thereof).

In some embodiments, when a standard of care treatment fails, such as when the mantle cell lymphoma is partially resistant to a chemotherapy, a second-line treatment is used that can include a well-known second-line treatment to treat mantle cell lymphoma. Accordingly, in some embodiments, the present invention provides a method of treating mantle cell lymphoma in a patient wherein the cancer is resistant to a first-line therapy, said method comprising administering X4P-001 optionally in combination with a second-line treatment.

In some embodiments, the present invention provides a method of treating a resistant mantle cell lymphoma comprising administering X4P-001 as the second-line treatment. In some embodiments, the present invention provides a method of treating a resistant mantle cell lymphoma comprising administering X4P-001 in combination with another second-line treatment or standard of care second-line treatment for mantle cell lymphoma (e.g., chemotherapy, immunotherapy, radioimmunotherapy (RIT), vaccination, autologous stem cell transplant, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat mantle cell lymphoma. In some embodiments, the present invention provides a method of treating a mantle cell lymphoma resistant to both first-line therapy and second-line therapy comprising administering X4P-001 as the third-line treatment. In some embodiments, the present invention provides a method of treating a mantle cell lymphoma resistant to both first-line therapy and second-line therapy comprising administering X4P-001 in combination with another third-line treatment or standard of care third-line treatment for mantle cell lymphoma (e.g., chemotherapy, immunotherapy, radioimmunotherapy (RIT), vaccination, autologous stem cell transplant, etc.).

In some embodiments, X4P-001 is administered as a sensitizer for the treatment of mantle cell lymphoma. Without wishing to be bound by any particular theory, it is believed that X4P-001 increases the efficacy of the standard of care, first-line, second-line, or third-line treatments for mantle cell lymphoma. In some embodiments, the present invention provides a method of treating a mantle cell lymphoma in a patient in need thereof, comprising administering X4P-001 to the patient prior to administration of one or more of a standard of care, first-line, second-line, or third-line treatment. In some embodiments, administration of X4P-001 results in a more effective treatment of the mantle cell lymphoma compared to treatment of mantle cell lymphoma in the absence of administration of X4P-001. In some embodiments, the present invention provides a method of treating a mantle cell lymphoma in a patient in need thereof, comprising administering X4P-001 to the patient after administration of one or more of a standard of care, first-line, second-line, or third-line treatment.

In some embodiments, the present invention provides a method of treating a mantle cell lymphoma in a patient in need thereof, comprising administering X4P-001 to the patient in combination with an additional therapeutic agent suitable for treating the mantle cell lymphoma. In some embodiments, the additional therapeutic agent is selected from ibrutinib, bortezomib, and acalabrutinib. In some embodiments, the additional therapeutic agent is selected from acalabrutinib (Calquence®; AstraZeneca), bortezomib (Velcade®; Takeda), and ibrutinib (Imbruvica®); Pharmacyclics/Janssen/AbbVie).

One of ordinary skill in the art will understand the amount and dosing regimen to administer such additional therapeutic agents for the treatment of mantle cell lymphoma. By way of example, the administration of exemplary therapeutic agents suitable for treating mantle cell lymphoma is summarized in Table 20, below.

TABLE 20
Exemplary Therapies for Mantle Cell Lymphoma

| Therapeutic Agent | Dosing regimen |
|---|---|
| acalabrutinib | 100 mg orally approximately every twelve hours; |
| (Calquence ®; AstraZeneca) | swallow whole with water and with or without food. |
| | Manage toxicities using treatment interruption, dose |
| | reduction, or discontinuation. |
| bortezomib | Recommended starting dose is 1.3 mg/m² administered |
| (Velcade ®; Takeda) | either as a 3 to 5 second bolus intravenous injection or |
| | subcutaneous injection. |
| | Use a lower starting dose for patients with moderate or |
| | severe hepatic impairment. |
| | Dose must be individualized to prevent overdose. |
| ibrutinib | 420-560 mg taken orally once daily until disease |
| (Imbruvica ®; | progression or unacceptable toxicity. |
| Pharmacyclics/Janssen/AbbVie) | Doses taken with a glass of water. |

### Bladder Cancer and Urothelial Carcinoma

In some embodiments, the present invention provides a method of treating bladder cancer or urothelial carcinoma in a patient in need thereof, comprising administering to the patient an effective amount of X4P-001 optionally in combination with one or more standard of care treatments, or a combination thereof, for bladder cancer or urothelial carcinoma.

Standard of care treatments for bladder cancer or urothelial carcinoma are well known to one of ordinary skill in the art and include electrocautery, radiotherapy, or chemotherapy, or a combination thereof. In some embodiments, the standard of care chemotherapy is selected from atezolizumab, avelumab, cisplatin, doxorubicin HCl, durvalumab, pembrolizumab, nivolumab, thiotepa, and valrubicin. In some embodiments, the additional therapeutic agent is selected from avelumab (Bavencio®; EMD Serono) and durvalumab (Imfinzi®; AstraZeneca).

In some embodiments, X4P-001 is administered to the patient as a monotherapy and as the first-line treatment for the bladder cancer or urothelial carcinoma. In other embodiments, X4P-001 is administered to the patient as a first-line treatment in combination with a standard of care treatment for bladder cancer or urothelial carcinoma (e.g., electrocautery, radiotherapy, or chemotherapy, or a combination thereof).

In some embodiments, when a standard of care treatment fails, such as when electrocautery fails to remove all cancerous tissue or the bladder cancer or urothelial carcinoma is partially resistant to a chemotherapy, a second-line treatment is used that can include a well-known second-line treatment to treat bladder cancer or urothelial carcinoma. Accordingly, in some embodiments, the present invention provides a method of treating bladder cancer or urothelial carcinoma in a patient wherein the cancer is resistant to a first-line therapy, said method comprising administering X4P-001 optionally in combination with a second-line treatment.

In some embodiments, the present invention provides a method of treating a resistant bladder cancer or urothelial carcinoma comprising administering X4P-001 as the second-line treatment. In some embodiments, the present invention provides a method of treating a resistant bladder cancer or urothelial carcinoma comprising administering X4P-001 in combination with another second-line treatment or standard of care second-line treatment for bladder cancer or urothelial carcinoma (e.g., radiotherapy, chemotherapy, immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat bladder cancer or urothelial carcinoma. In some embodiments, the present invention provides a method of treating a bladder cancer or urothelial carcinoma resistant to both first-line therapy and second-line therapy comprising administering X4P-001 as the third-line treatment. In some embodiments, the present invention provides a method of treating a bladder cancer or urothelial carcinoma resistant to both first-line therapy and second-line therapy comprising administering X4P-001 in combination with another third-line treatment or standard of care third-line treatment for bladder cancer or urothelial carcinoma (e.g., radiotherapy, chemotherapy, immunotherapy, etc.).

In some embodiments, X4P-001 is administered as a sensitizer for the treatment of bladder cancer or urothelial carcinoma. Without wishing to be bound by any particular theory, it is believed that X4P-001 increases the efficacy of the standard of care, first-line, second-line, or third-line treatments for bladder cancer or urothelial carcinoma. In some embodiments, the present invention provides a method of treating a bladder cancer or urothelial carcinoma in a patient in need thereof, comprising administering X4P-001 to the patient prior to administration of one or more of a standard of care, first-line, second-line, or third-line treatment. In some embodiments, administration of X4P-001 results in a more effective treatment of the bladder cancer or urothelial carcinoma compared to treatment of bladder cancer or urothelial carcinoma in the absence of administration of X4P-001. In some embodiments, the present invention provides a method of treating a bladder cancer or urothelial carcinoma in a patient in need thereof, comprising administering X4P-001 to the patient after administration of one or more of a standard of care, first-line, second-line, or third-line treatment.

In some embodiments, the present invention provides a method of treating a bladder cancer or urothelial carcinoma in a patient in need thereof, comprising administering X4P-001 to the patient in combination with an additional therapeutic agent suitable for treating the bladder cancer or urothelial carcinoma. In some embodiments, the additional therapeutic agent is selected from atezolizumab, avelumab, cisplatin, doxorubicin HCl, durvalumab, pembrolizumab, nivolumab, thiotepa, and valrubicin. In some embodiments, the additional therapeutic agent is selected from avelumab (Bavencio®; EMD Serono) and durvalumab (Imfinzi®; AstraZeneca).

One of ordinary skill in the art will understand the amount and dosing regimen to administer such additional therapeutic agents for the treatment of bladder cancer or urothelial carcinoma. By way of example, the administration of exemplary therapeutic agents suitable for treating bladder cancer or urothelial carcinoma is summarized in Table 21, below.

TABLE 21
Exemplary Therapies for Bladder Cancer or Urothelial Carcinoma

| Therapeutic Agent | Dosing regimen |
|---|---|
| avelumab | 10 mg/kg as an intravenous infusion over 60 minutes every 2 |
| (Bavencio ®; EMD Serono) | weeks. |
| | Pre-medicate with acetaminophen and an antihistamine for the |
| | first 4 infusions and subsequently as needed. |
| durvalumab | 10 mg/kg as an intravenous infusion over 60 minutes every 2 |
| (Imfinzi ®; AstraZeneca). | weeks, until disease progression or unacceptable toxicity. |

### Merkel Cell Carcinoma

In some embodiments, the present invention provides a method of treating Merkel cell carcinoma in a patient in need thereof, comprising administering to the patient an effective amount of X4P-001 optionally in combination with one or more standard of care treatments, or a combination thereof, for Merkel cell carcinoma.

Standard of care treatments for Merkel cell carcinoma are well known to one of ordinary skill in the art and include surgery, radiotherapy, or chemotherapy, or a combination thereof. In some embodiments, the standard of care chemotherapy is avelumab (Bavencio®; EMD Serono). In some embodiments, the additional therapeutic agent is avelumab (Bavencio®; EMD Serono).

In some embodiments, X4P-001 is administered to the patient as a monotherapy and as the first-line treatment for the Merkel cell carcinoma. In other embodiments, X4P-001 is administered to the patient as a first-line treatment in combination with a standard of care treatment for Merkel cell carcinoma (e.g., surgery, radiotherapy, or chemotherapy, or a combination thereof).

In some embodiments, when a standard of care treatment fails, such as when surgery fails to remove all cancerous tissue or the Merkel cell carcinoma is partially resistant to a chemotherapy, a second-line treatment is used that can include a well-known second-line treatment to treat Merkel cell carcinoma. Accordingly, in some embodiments, the present invention provides a method of treating Merkel cell carcinoma in a patient wherein the cancer is resistant to a first-line therapy, said method comprising administering X4P-001 optionally in combination with a second-line treatment.

In some embodiments, the present invention provides a method of treating a resistant Merkel cell carcinoma comprising administering X4P-001 as the second-line treatment. In some embodiments, the present invention provides a method of treating a resistant Merkel cell carcinoma comprising administering X4P-001 in combination with another second-line treatment or standard of care second-line treatment for Merkel cell carcinoma (e.g., radiotherapy, chemotherapy, immunotherapy, etc.). In some embodiments, the second-line treatment is selected from a chemotherapy.

In some instances when the first-line or second-line standard of care treatment fails, such as when chemotherapy continues to fail and remission occurs, a third-line treatment is administered to the patient that can include a well-known third-line treatment to treat Merkel cell carcinoma. In some embodiments, the present invention provides a method of treating a Merkel cell carcinoma resistant to both first-line therapy and second-line therapy comprising administering X4P-001 as the third-line treatment. In some embodiments, the present invention provides a method of treating a Merkel cell carcinoma resistant to both first-line therapy and second-line therapy comprising administering X4P-001 in combination with another third-line treatment or standard of care third-line treatment for Merkel cell carcinoma (e.g., radiotherapy, chemotherapy, immunotherapy, etc.).

In some embodiments, X4P-001 is administered as a sensitizer for the treatment of Merkel cell carcinoma. Without wishing to be bound by any particular theory, it is believed that X4P-001 increases the efficacy of the standard of care, first-line, second-line, or third-line treatments for Merkel cell carcinoma. In some embodiments, the present invention provides a method of treating a Merkel cell carcinoma in a patient in need thereof, comprising administering X4P-001 to the patient prior to administration of one or more of a standard of care, first-line, second-line, or third-line treatment. In some embodiments, administration of X4P-001 results in a more effective treatment of the Merkel cell carcinoma compared to treatment of Merkel cell carcinoma in the absence of administration of X4P-001. In some embodiments, the present invention provides a method of treating a Merkel cell carcinoma in a patient in need thereof, comprising administering X4P-001 to the patient after administration of one or more of a standard of care, first-line, second-line, or third-line treatment.

In some embodiments, the present invention provides a method of treating a Merkel cell carcinoma in a patient in need thereof, comprising administering X4P-001 to the patient in combination with an additional therapeutic agent suitable for treating the Merkel cell carcinoma. In some embodiments, the additional therapeutic agent is avelumab (Bavencio®; EMD Serono). In some embodiments, the additional therapeutic agent is avelumab (Bavencio®; EMD Serono).

One of ordinary skill in the art will understand the amount and dosing regimen to administer such additional therapeutic agents for the treatment of Merkel cell carcinoma. By way of example, the administration of exemplary therapeutic agents suitable for treating Merkel cell carcinoma is summarized in Table 22, below.

TABLE 22
Exemplary Therapies for Merkel Cell Carcinoma

| Therapeutic Agent | Dosing regimen |
|---|---|
| avelumab | 10 mg/kg as an intravenous infusion over 60 minutes every 2 |
| (Bavencio ®; EMD Serono) | weeks. |
| | Pre-medicate with acetaminophen and an antihistamine for |
| | the first 4 infusions and subsequently as needed. |

In some embodiments, the present invention provides a method of treating a cancer in a patient in need thereof, as described herein, comprising administering to the patient X4P-001 in combination with one or more additional therapies wherein the combination of X4P-001 and the one or more additional therapies acts synergistically. In some embodiments, the administration of X4P-001 in combination with an additional therapeutic agent results in a reduction of the effective amount of that additional therapeutic agent as compared to the effective amount of the additional therapeutic agent in the absence of administration in combination with X4P-001. In some embodiments, the effective amount of the additional therapeutic agent administered in combination with X4P-001 is about 90%, about 80%, about 70%, about 60%, about 50%, about 40%, about 30%, about 20%, or about 10% of the effective amount of the additional therapeutic agent in the absence of administration in combination with X4P-001.

### Dosage and Formulations

X4P-001 is a CXCR4 antagonist, with molecular formula C₂₁H₂₇N₅; molecular weight 349.48 amu; and appearance as a white to pale yellow solid. Solubility: X4P-001 is freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water. Melting point: 108.9° C.

The chemical structure of X4P-001 is depicted below.

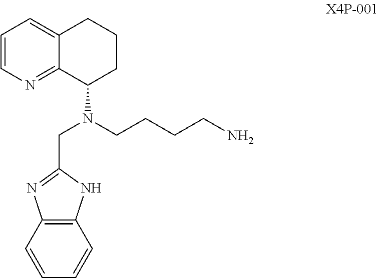 [SMILES: NCCCCN(Cc1nc2ccccc2[nH]1)[C@H]1CCCc2cccnc21]

In certain embodiments, a pharmaceutical composition containing X4P-001 or a pharmaceutically acceptable salt thereof is administered orally in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of X4P-001 has been generally determined to be between about 12 to about 24 hours, or approximately 14.5 hrs. Dosage for oral administration may be from about 100 mg to about 1200 mg once or twice per day. In certain embodiments, the dosage of X4P-001 or a pharmaceutically acceptable salt thereof useful in the invention is from about 200 mg to about 600 mg daily. In other embodiments, the dosage of X4P-001 or a pharmaceutically acceptable salt thereof useful in the invention may range from about 400 mg to about 800 mg, from about 600 mg to about 1000 mg or from about 800 mg to about 1200 mg daily. In certain embodiments, the invention comprises administration of an amount of X4P-001 or a pharmaceutically acceptable salt thereof of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg.

In some embodiments, a provided method comprises administering to the patient a pharmaceutically acceptable composition comprising X4P-001 or a pharmaceutically acceptable salt thereof wherein the composition is formulated for oral administration. In certain embodiments, the composition is formulated for oral administration in the form of a tablet or a capsule. In some embodiments, the composition comprising X4P-001 or a pharmaceutically acceptable salt thereof is formulated for oral administration in the form of a capsule.

In certain embodiments, a provided method comprises administering to the patient one or more unit doses, such as capsules, comprising 100-1200 mg X4P-001 or a pharmaceutically acceptable salt thereof as an active ingredient; and one or more pharmaceutically acceptable excipients.

A composition according to the present invention comprises a compound for use in the invention or a pharmaceutically acceptable salt or derivative thereof and a pharmaceutically acceptable carrier, adjuvant, or vehicle. The amount of compound in compositions of this invention is an amount effective to measurably inhibit CXCR4, or a mutant thereof, in a biological sample or in a patient. In certain embodiments, a composition of this invention is formulated for administration to a patient in need of such a composition. In some embodiments, a composition of this invention is formulated for oral administration to a patient.

The term “patient,” as used herein, means an animal, preferably a mammal, and most preferably a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a patient, is capable of providing, either directly or indirectly, a compound of this invention.

Compositions of the present invention may be administered orally, parenterally, by inhalation spray, topically (as by powders, ointments, or drops), rectally, nasally, buccally, intravaginally, intracisternally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The compounds and compositions, according to the method of the present invention, may be administered using any amount and any route of administration effective for treating a cancer, such as those disclosed herein. The exact amount required will vary from subject to subject, depending on the species, age, and general condition of the subject, the severity of the cancer, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the cancer being treated and the severity of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the present invention provides a pharmaceutical composition comprising X4P-001 or a pharmaceutically acceptable salt thereof, one or more diluents, a disintegrant, a lubricant, a flow aid, and a wetting agent. In some embodiments, the present invention provides a composition comprising 10-1200 mg X4P-001 or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001 or a pharmaceutically acceptable salt thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day. In some embodiments, the unit dosage form comprises a capsule containing about 25 mg, about 50 mg, about 75 mg, about 100 mg, about 150 mg, or about 200 mg of X4P-001 or a pharmaceutically acceptable salt thereof.

In some embodiments, the present invention provides a unit dosage form comprising a pharmaceutical composition comprising:
(a) X4P-001, or a pharmaceutically acceptable salt thereof—about 30-40% by weight of the composition;
(b) microcrystalline cellulose—about 20-25% by weight of the composition;
(c) dibasic calcium phosphate dihydrate—about 30-35% by weight of the composition;
(d) croscarmellose sodium—about 5-10% by weight of the composition;
(e) sodium stearyl fumarate—about 0.5-2% by weight of the composition;
(f) colloidal silicon dioxide—about 0.1-1.0% by weight of the composition; and
(g) sodium lauryl sulfate—about 0.1-1.0% by weight of the composition.

In some embodiments, the present invention provides a unit dosage form comprising a composition comprising:
(a) X4P-001, or a pharmaceutically acceptable salt thereof—about 37% by weight of the composition;
(b) microcrystalline cellulose—about 23% by weight of the composition;
(c) dibasic calcium phosphate dihydrate—about 32% by weight of the composition;
(d) croscarmellose sodium—about 6% by weight of the composition;
(e) sodium stearyl fumarate—about 1% by weight of the composition;
(f) colloidal silicon dioxide—about 0.3% by weight of the composition; and
(g) sodium lauryl sulfate—about 0.5% by weight of the composition.

In some embodiments, the present invention provides a unit dosage form comprising a composition comprising:
(a) X4P-001, or a pharmaceutically acceptable salt thereof—about 55-65% by weight of the composition;
(b) microcrystalline cellulose—about 10-15% by weight of the composition;
(c) dibasic calcium phosphate dihydrate—about 15-20% by weight of the composition;
(d) croscarmellose sodium—about 5-10% by weight of the composition;
(e) sodium stearyl fumarate—about 0.5-2% by weight of the composition;
(f) colloidal silicon dioxide—about 0.1-1.0% by weight of the composition; and
(g) sodium lauryl sulfate—about 0.1-1.0% by weight of the composition.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1—Measurement of CD8+ T Cells

Assessment of the effectiveness of the present invention can be made in part by measurement of the CD8+ T cell population. Expanding or increasing the density of tumor infiltrating lymphocytes, especially CD8+ T cells, can help kill tumor cells. Dudley et al., (2010) Clin. Cancer Research, 16:6122-6131. CD8+ T cells can be detected, isolated and quantified utilizing methods described in Herr et al., (1996), J. Immunol. Methods 191:131-142; Herr et al., (1997) J. Immunol. Methods 203:141-152; and Scheibenbogen et al., (2000) J Immunol. Methods 244:81-89. The full disclosure of each of these publications is hereby incorporated by reference herein.

### Example 2—Renal Cell Carcinoma Xenograft Model

In order to assess the effects of the present invention on renal cell carcinoma, a human RCC xenograft model can be used, as described in Pavia-Jimenez et al. (2014) Nature Protocols 9:1848-1859; Grisanzio et al. (2011) J Pathol 225:212-221. The full disclosure of each of these publications is hereby incorporated by reference herein.

### Example 3—Criteria for Evaluating Response in Patients with Solid Tumors

The response of patients with solid tumors to treatment can be evaluated using the criteria set forth in RECIST 1.1, Eisenhauer et al., (2009) Eur. J. Cancer, 45:228-247, the full disclosure of which is hereby incorporated by reference herein.

### Example 4—Cytokine and Chemokine Studies

The in vivo effects of treatment with X4P-001 and nivolumab on chemokine production by RCC cells are assessed as follows:

Tumors excised from the mice undergoing treatment with X4P-001 and nivolumab in Example 1 and 2 are analyzed by RT-PCR for drug-induced changes in the expression of M-CSF (CSF-1), CXCL1 (MGSA/gro-), CXCL2 (MIP-2/gro-), MIP-2/gro-, CXCLS (ENA-78), CXCL6 (GCP-2), CXCL8 (IL-8), GM-CSF, VEGF, TNF, CCL22, and CCL28. The various ELR-containing CXCL chemokines listed are known to activate CXCR2 (Gale and McColl (1999) BioEssays 21: 17-28), a chemokine receptor recently implicated in MDSC recruitment (Highfill et al. (2014) Sci Transl Med 6: ra67). The cytokines VEGF, GM-CSF, and TNF are also thought to mediate MDSC chemotaxis into tumor tissue. CCL22 and CCL28 have been likewise implicated in the recruitment of Tregs (Facciabene et al. (2011), Nature 475: 226-230; Montane et al. (2011) J Clin Invest 2011; 121: 3024-8).

Numerous chemokines and other inflammatory mediators have been shown to regulate the trafficking of MDSC into tumor tissue (Highfill et al. (2014) Sci Transl Med 6: ra67; Acharyya et al. (2012) Cell 150:165-7813; Zhao et al. (2012) Clin Invest 122: 4094-4104). To determine which chemokines/cytokines are responsible for the influx of MDSC into RCC during treatment with VEGF-targeted therapies, CD11b+/Gr-1+MDSC are isolated from the spleens of tumor-bearing mice undergoing treatment with nivolumab. The MDSC are then infected with a small pooled lentiviral shRNA library (DeCode GIPZ, Thermo Scientific) for a select group of G protein-coupled and other receptors known to regulate MDSC trafficking. The library will include shRNAs for TNFR-1 and -2, IL-4R, and whole array of CXCR and CCR chemokine receptors (CXCR1-5, CCR 1-9). Several of these (e.g. CXCR-1, -2, and -4) engage chemokines known to promote MDSC recruitment (Highfill et al. (2014) Sci Transl Med 6: ra67; Acharyya et al. (2012) Cell 150:165-7813; Zhao et al. (2012) Clin Invest 122: 4094-4104).

Example 5—Clinical Treatment Regimen

Treatment with X4P-001 as a monotherapy, or in combination with a checkpoint inhibitor, such as nivolumab, may be performed in cycles, such as on a 2 week, 4 week, 6 week or 8 week cycle. In certain embodiments, the cycle is 4 weeks long. X4P-001 at a determined dose from 200 mg to 1200 mg daily is administered orally either once daily or twice daily in divided doses. Patients are instructed about both dosing schedule and requirements relating to food or drink near the time of dosing.

Dosing Schedule.

The daily dose is taken first thing in the morning. Where the dose is divided, the first daily dose is taken in the morning and the second daily dose approximately 12 hours later using the following guidelines:
Dosing should be at the same time(s) each day ±2 hr.
For twice daily dosing, the interval between successive doses should not be <9 hours nor >15 hours. If the interval would be >15 hrs, the dose should be omitted and the usual schedule resumed at the next dose.
Restrictions relating to food. Absorption is impacted by food and patients will be instructed as follows:
For the morning dose
   No food or drink (except water) after midnight until the time of dosing
   No food or drink (except water) for 2 hour after dosing.
For the second daily dose, if applicable
   No food or drink (except water) for 1 hour before dosing
   No food or drink (except water) for 2 hours after dosing.

Nivolumab is administered consistent with prescribed labeling information. Concomitant treatment with X4P-001 and nivolumab may be administered, beginning with daily administration of X4P-001 at day 1. Initial treatment with nivolumab is at 3 mg/kg administered by intravenous infusion over 60 minutes in clinic at the week 4 and 7 visits. Patients may, with the approval of their clinician, vary the dosing schedule or dosage of nivolumab.

Dosing of X4P-001 and/or nivolumab may be adjusted by the clinician as appropriate. The dose of X4P-001 and/or nivolumab may be lowered according to the judgment of the clinician. If a patient receiving X4P-001 in combination with nivolumab experiences an adverse event at Grade >2, the dose of X4P-001 and/or nivolumab may be lowered according to the judgment of the clinician. If a patient successfully completes the first 4 weeks of treatment, that is, without experiencing any adverse events greater than Grade 2, the daily dose of X4P-001 and/or nivolumab may be increased, consistent with the judgment of the clinician.

Evaluation of Response to Treatment and Disease Status.

Classification of tumor response may be performed according to codified tumor response evaluation, according to the Response Evaluation Criteria in Solid Tumors Group (“RECIST”), as described in Therasse et al. (2000), J. National Cancer Institute, 92:205-216. Radiologic assessment of ccRCC is accomplished by Computed Tomography (CT) with slice thickness ≤5 mm and contrast. CT is performed prior to treatment (baseline) and may be made at intervals during treatment to determine the response.

Key terminology:
Measurable non-nodal lesions—≥10 mm in longest diameter.
Measurable nodal lesions—≥15 mm in short axis
Nonmeasurable lesions—lesions that are smaller, including those that cannot be measured.
Measurable disease—presence of at least one measurable lesion.

### Target Lesions

At baseline, four (4) measureable lesions, two (2) for each individual organ, are identified, documented, and the appropriate diameter of each is recorded. If measurable extra-renal lesions are present, a measurable extra-renal lesion is also identified, documented, and the appropriate diameter is recorded. Lesions are selected based on size, to be representative of disease, and suitable for reproducible repeat measurement. Target lesions may include measurable lymph nodes.

During treatment, each target lesion is assessed for Complete Response, Partial Response, Stable Disease, or Progressive Disease as follows:

Complete Response (CR)
(a) Disappearance of all non-nodal lesions, and
(b) Absence of pathologic lymph nodes^{a}.

Partial Response (PR)
(a) ≥30% decrease from baseline in the SOD of the target lesions

Stable Disease (SD)
(a) Persisting disease that does not meet criteria for either PR or PD

Progressive Disease (PD)
a) ≥20% increase in the SOD of the target lesions, compared to the smallest sum, which may be either at baseline or while on treatment; and
(b) an absolute increase of ≥5 mm in the SOD.

### Non-Target Lesions

All other lesions present at baseline, including pathologic nodes (defined as nodes >10 mm in short axis) should be documented (quantitative measurements are not required) so that they can be classified on follow-up as present, absent, or unequivocal progression.

Complete Response (CR)
(a) Disappearance of all non-target lesions, and
(b) Absence of pathologic lymph nodes^{a}.

Non-CR/Non-PD
Persistence of one or more non-target lesions

Progressive Disease (PD)
Unequivocal progression of existing non-target lesions.
   [Note: a=All lymph nodes, whether or not designated target or non-target lesions, have short axis diameter ≤10 mm]

### New Lesions

A new lesion should be unequivocal (e.g., not attributable to variation in technique); includes lesions in a location not scanned at baseline.

### Pharmacokinetic Assessments

If desired, pharmacokinetic assessment of blood samples for plasma levels of X4P-001 and nivolumab may be conducted. Blood samples are collected as scheduled. Samples are analyzed for X4P-001 concentration using reversed-phase high performance liquid chromatography (RP-HPLC) with MS/MS detection. The validated range of this bioanalytic method is 1 to 5,000 ng/mL in plasma.

Pharmacokinetic assessment of nivolumab may be accomplished using techniques, such as those described in Glassman and Balthasar (2014) Cancer Biol. Med. 11:20-33; Wang et al. (2014), Cancer Immunology Research, 2:1-11; or the Assessment Report of the European Medicines Agency (EMA) for nivolumab EMEA, assessment report EMA/CHMP/76688/2015, Apr. 23, 2015. The full disclosure of these documents are hereby specifically incorporated herein by reference.

### Example 6—Extended Survival of Mice Treated with CXCR4 Inhibitor and Anti-PD1 in a Syngeneic Mouse Tumor Model (MC38)

Treatment with a CXCR4 inhibitor such as X4P-001 in combination with anti-PD-1 antibody was tested to determine whether the combination would reduce MDSC and improve the CD8+/Treg ratio of tumor infiltrating lymphocytes.

Mice were treated as follows:

| Group | n | Treatment |
|---|---|---|
| Group 1 | 12 | Control (Vehicle + Rat IgG2a); |
| Group 2 | 12 | X4P-001 (100 mg/kg, PO, QD) + Rat IgG2a (5 mg/kg, Days 1, 4, 7, 11) |
| Group 3 | 12 | Vehicle + anti-PD-1 (5 mg/kg, Days 1, 4, 7, 11) |
| Group 4 | 12 | X4P-001 + anti-PD1 |
| Group 5 | 3 | Control (Vehicle + Rat IgG2a) |
| Group 6 | 3 | X4P-001 (100 mg/kg, PO, QDx8) + |
| | | Rat IgG2a (5 mg/kg, Days 1, 4, 7, 11) |
| Group 7 | 3 | Vehicle +anti-PD-1 (5 mg/kg, Days 1, 4, 7, 11) |
| Group 8 | 3 | X4P-001 + anti-PD1 |

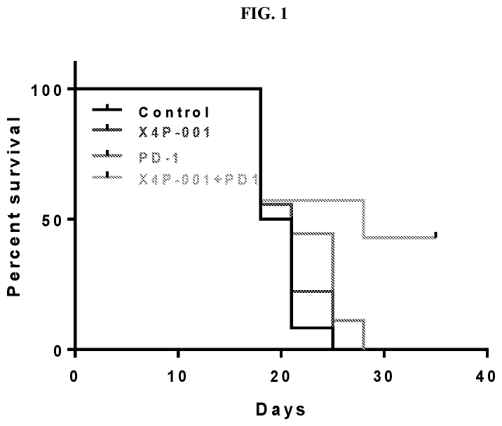

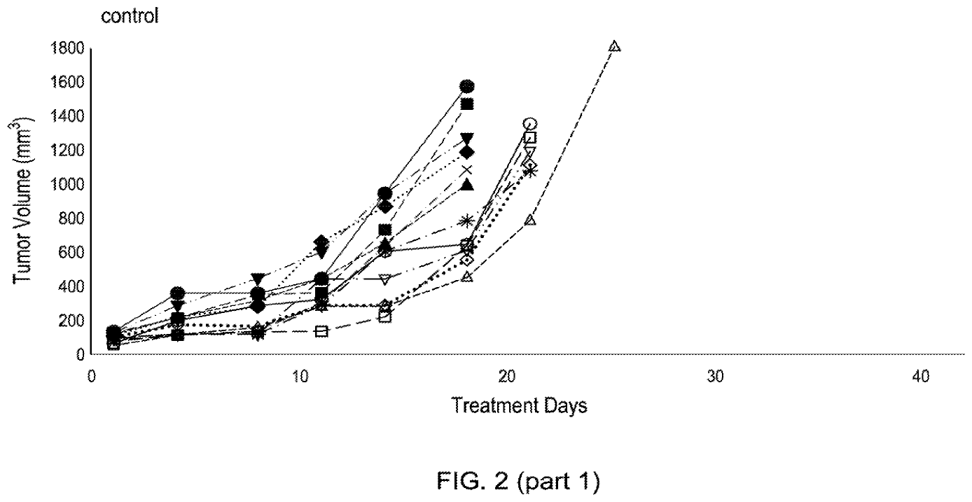

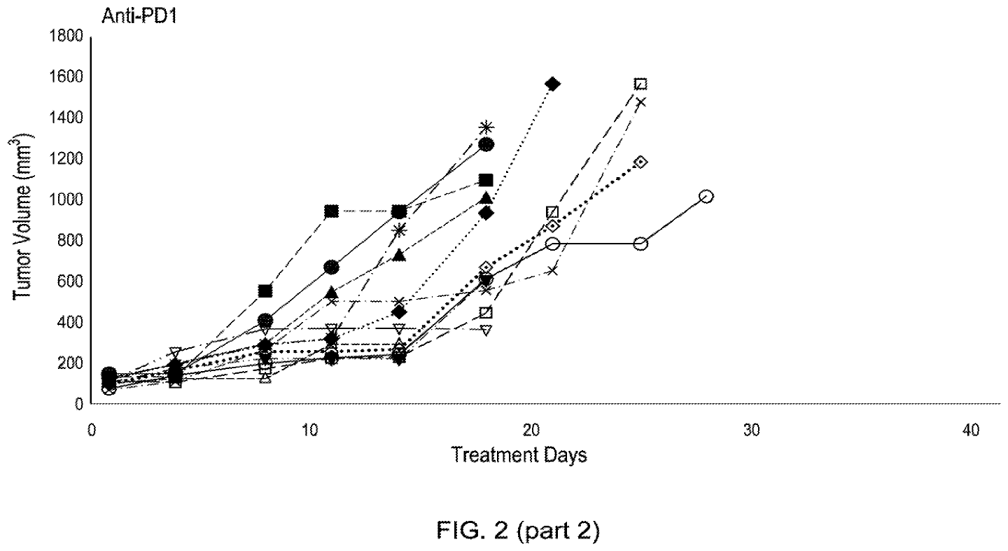

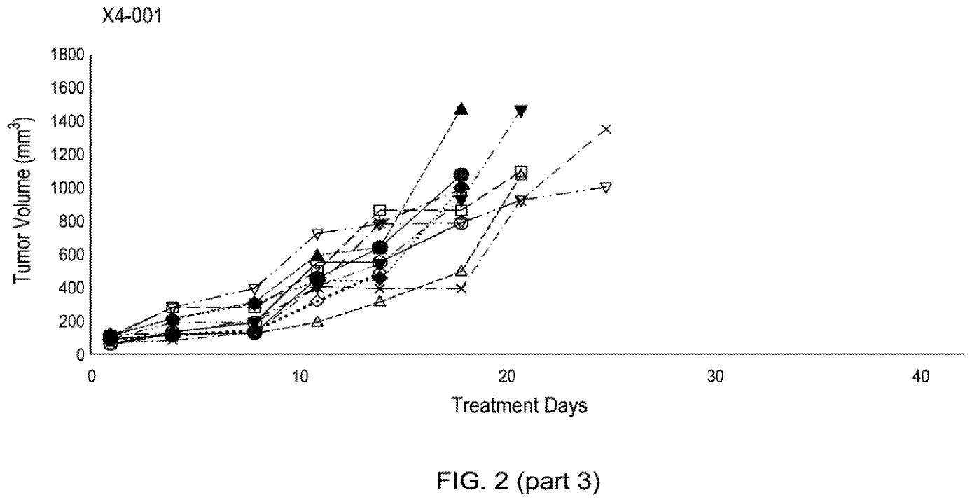

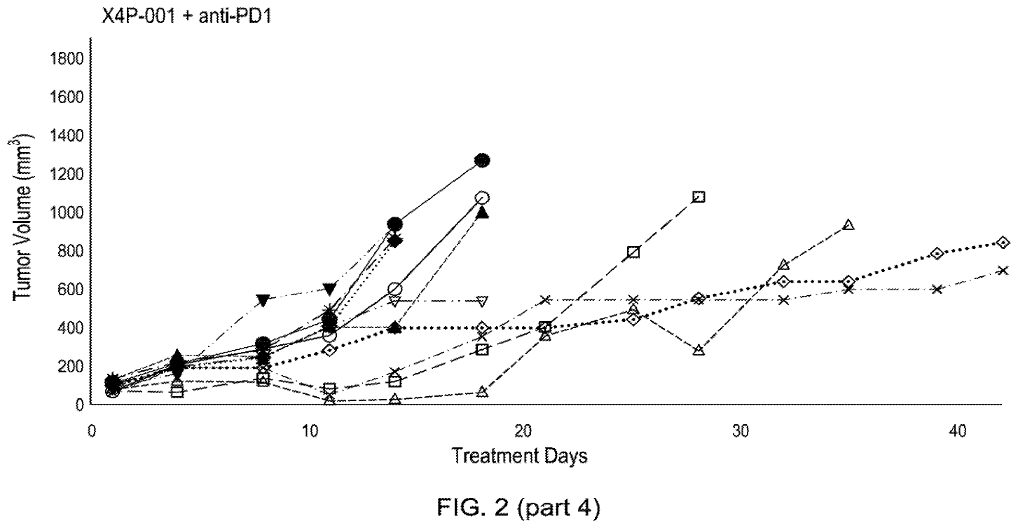

The endpoint of the experiment was either (a) tumor volume of 1000 mm³ or (b) 45 days, whichever comes first. Responders can be followed for longer than 45 days. As shown in FIG. 1, the results of this experiment for Groups 1 through 4 demonstrate enhanced activity for the combination therapy, greatly extending the survival of the combination therapy group to nearly 50% after 35+ days. As shown in FIG. 2, the combination therapy also controlled the tumor volume in some mice compared with either X4P-001 or nivolumab alone.

On Day 8, tumor samples from Groups 5-8 are obtained and divided into three parts. The first part is process to single cells by flow cytometry; second part is preserved by snap freeze; and the third part is preserved as formalin-fixed, paraffin-embedded (FFPE) blocks for IHC analysis of biomarkers.

Samples subjected to flow cytometry are sorted into the following cell types:

| Cell | |
|---|---|
| Population | Signature Marker |
| CD4 | CD3⁺ CD4⁺ CD8⁻ |
| CD8 | CD3⁺ CD4⁻ CD8⁺ |
| Tregs | CD3⁺ CD4⁺ CD25⁺ FoxP3⁺ |
| MDSC | CD3⁻ CD11b⁺ GR-1⁺ |

The results of flow cytometry are depicted in the table below:

| | Percent of CD45⁺ Population (Mean ± SEM) | | | | |
|---|---|---|---|---|---|
| Cell Type | CD4⁺ | CD8⁺ | T_{reg} | MDSC | CD8⁺/Treg |
| Control | 1.21 ± 0.29 | 3.91 ± 0.69 | 0.5 ± 0.08 | 28.97 ± 3.6 | 7.82 |
| X4P-001 | 1.3 ± 0.51 | 6.11 ± 0.66 | 0.49 ± 0.14 | 16.2 ± 0.67 | 12.47 |
| Anti-PD-1 | 2.56 ± 0.32 | 8.25 ± 2.53 | 0.57 ± 0.25 | 25.47 ± 5.25 | 14.47 |
| X4P-001+30 | 1.38 ± 0.17 | 5.52 ± 1.03 | 0.5 ± 0.05 | 21.06 ± 7.89 | 11.04 |
| anti-PD-1 | | | | | |

X4P-001 exhibited a significant effect on reducing MDSCs and increasing CD8⁺ T cells, while anti-PD-1 significantly increased both CD4⁺ and CD8⁺ T cell populations. Each of the monotherapy arms and the combination arm increased the CD8⁺/T_{reg} ratio, which has been described as predictive of therapeutic efficacy in a number of cancer models. (Sato et al. (2005) PNAS 102:1538-18543; Gao et al. (2007) J. Clin. Oncol. 25:2586-2593; Curran et al. (2009) PNAS 107:4275-4280; Mardiana et al. (2017) Cancer Res. 77:1296-1309).

### Example 7—Immunohistochemical Analysis of Tumor Tissue Samples From Human Patients With Various Cancer Types

Formalin-fixed paraffin-embedded (FFPE) tumor tissue samples were obtained and immunostained using an anti-CXCR4 antibody, and scored for expression of CXCR4.

Tissue samples from twenty (20) patients with adrenocortical adenocarcinoma (10 malignant tumors; 10 benign tumors) were screened for expression of CXCR4 and CXCL12. 20/20 tissue samples expressed CXCR4; 5/10 malignant tissue samples expressed CXCL12; 0/10 benign tumor tissue samples expressed CXCL12.

Fifty-four (54) tissue samples from eighteen (18) patients (3 samples each) with pancreatic duct adenocarcinoma (all malignant) were screened for expression of CXCR4 and CXCL12. 25/54 malignant tissue samples expressed CXCR4; 2/54 malignant tissue samples expressed CXCL12.

Six (6) tissue samples from two patients (3 samples each) with islet cell carcinoma (all malignant) were screened for expression of CXCR4 and CXCL12. 6/6 malignant tissue samples expressed CXCR4; 6/6 malignant tissue samples expressed CXCL12.

Six (6) tissue samples from two patients (3 samples each) with pancreatic cancer (all malignant) were screened for expression of CXCR4 and CXCL12. 2/6 malignant tissue samples expressed CXCR4; 6/6 malignant tissue samples expressed CXCL12.

Twenty-one (21) samples from sixteen (16) patients with gallbladder carcinoma (5 hyperplasia (2 samples each); and 11 malignant (1 papillary; 5 squamous cell carcinoma; 5 adenocarcinoma) were screened for expression of CXCR4 and CXCL12. 7/10 hyperplasia tissue samples; and 9/11 malignant tissue samples expressed CXCR4; 3/9 hyperplasia tissue samples and 5/11 malignant tissue samples expressed CXCL12 (1 hyperplasia tumor had insufficient tissue remaining for second sample).

Seventy (70) samples from thirty-five (35) patients with glioblastoma (2 samples each), all malignant; and ten (10) normal brain tissue samples from five (5) patients were screened for expression of CXCR4 and CXCL12. 36/70 malignant glioblastoma tissue samples expressed CXCR4; 38/70 malignant glioblastoma tissue samples expressed CXCL12. None of the normal brain tissue samples (0/10) expressed either CXCR4 or CXCL12.

Tissue samples from sixty-four (64) patients with hepatocholaniocarcinoma (all malignant) were screened for expression of CXCR4 and CXCL12. 33/63 tissue samples expressed CXCR4 (1 tumor had insufficient tissue for sampling); 2/61 tissue samples expressed CXCR4 (3 tumors had insufficient tissue for sampling).

Sixty (60) tissue samples from twenty (20) patients with medulloblastoma (3 samples each), all malignant; and normal brain tissue samples from three (3) patients, were screened for expression of CXCR4 and CXCL12. 56/60 malignant medulloblastoma samples expressed CXCR4; 2/60 malignant medulloblastoma samples expressed CXCL12. None of the normal brain tissue samples (0/3) expressed either CXCR4 or CXCL12.

### Example 8—Immunohistochemical Analysis of Intra-Tumor T-Cell Infiltrates in Tissue Samples From Human Melanoma Patient Before and After Treatment With CXCR4 Inhibitor X4P-001

Intra-tumoral tissue samples were obtained from a patient with melanoma prior to treatment (Day 1), and after three (3) weeks of treatment with CXCR4 inhibitor X4P-001 (200 mg BID, oral) (Week 4). Samples were stained for CD8+, indicative of activated T-cells, and FoxP3+, indicative of immunosuppressive regulator T-cells (Tregs). The tables below indicate the total counts and density per square millimeter (mm²) of CD8+ activated T-cells and FoxP3+ Tregs, as well as the ratio of CD8+ activated T-cells:FoxP3+ Tregs per mm² at day 1 and after 3 weeks of treatment.

| | Total CD8+ | Total CD8 Area | Total CD8+/mm² |
|---|---|---|---|
| Day 1 | 720 | 12.659195 | 56.87565442 |
| Week 4 | 1685 | 13.875068 | 121.4408463 |

| | Total FoxP3+ | Total FoxP3 Area | Total FoxP3+/mm² |
|---|---|---|---|
| Day 1 | 313 | 12.7304 | 24.5868158 |
| Week 4 | 337 | 13.372556 | 25.2008666 |

| | CD8+:FoxP3+ Ratio |
|---|---|
| Day 1 | 2.31325825 |
| Week 4 | 4.81891548 |

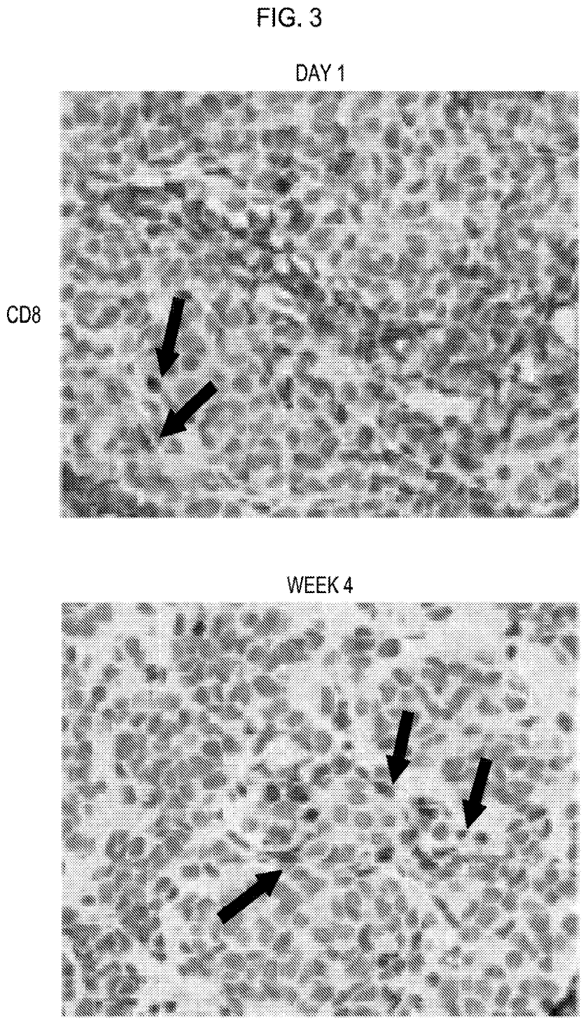

As demonstrated in the Tables above, after 3 weeks of treatment with CXCR4 inhibitor X4P-001 (200 mg BID, oral), a marked increase in intra-tumoral CD8+ activated T-cell counts was observed. Further, the CD8+/Treg ratio increased by >2-fold. FIG. 3 shows representative images of the tumor stained for CD8+ T-cell counts.

### REFERENCES

1. Ratajczak, et al. The pleotropic effects of the SDF-1—CXCR4 axis in organogenesis, regeneration, and tumorigenesis. Leukemia 2006:20; 1915-1924.
2. Scala, et al. Expression of CXCR4 predicts poor prognosis in patients with malignant melanoma. Clin Cancer Res 2005:11; 1835-1841.
3. Toyozawa, et al. Chemokine receptor CXCR4 is a novel marker for the progression of cutaneous malignant melanoma. Acta Histochem Cytochem. 2012; 45:293-299.
4. Kim, et al. CXCR4 signaling regulates metastasis of chemoresistant melanoma cells by a lymphatic metastatic niche. Cancer Res. 2010; 70:10411-10421.
5. Mosi R M, Anastassova V, Cox J, et al. The molecular pharmacology of AMD11070: An orally bioavailable CXCR4 HIV entry inhibitor. Biochem Pharmacol. 2012; 83:472-479.
6. D'Alterio, et al. Inhibition of stromal CXCR4 impairs development of lung metastases. Cancer Immunol Immunother. 2012:61; 1713-1720.
7. Feig, et al. Targeting CXCL12 from FAP-expressing carcinoma-associated fibroblasts synergizes with anti-PD-L1 immunotherapy in pancreatic cancer. PNAS 2013; 110:20212-20217.
9. Stone, et al. Multiple-Dose Escalation Study of the Safety, Pharmacokinetics, and Biologic Activity of Oral AMD070, a Selective CXCR4 Receptor Inhibitor, in Human Subjects. Antimicrob Agents Chemother. 2007; 51(7):2351-2358.
10. Moyle, et al. Proof of Activity with AMD11070, an Orally Bioavailable Inhibitor of CXCR4-Tropic HIV Type 1. Clin Infect Dis. 2009; 48:798-805.
12. Tumeh, et al. PD-1 blockade induces responses by inhibiting adaptive immune resistance. Nature 2014:515; 568-571.
14. Nyunt, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47:559-565.
15. Cao, et al. Effect of Low-Dose Ritonavir on the Pharmacokinetics of the CXCR4 Antagonist AMD070 in Healthy Volunteers. Antimicrob Agents Chemother. 2008; 52:1630-1634.
16. Common Terminology Criteria for Adverse Events (CTCAE). Version 4.0, 28 May 2009. U.S. Department of Health and Human Services, National Institutes of Health, National Cancer Institute. NIH Publication No. 03-5410.
17. NCI CTCAE v4.03, 14 Jun. 2010 available at (accessed 6 Apr. 2015): http://evs.nci.nih.gov/ftp1/CTCAE/CTCAE_4.03_2010-06-14_QuickReference_5x7.pdf
18. WMA Declaration of Helsinki—Ethical Principles for Medical Research Involving Human Subjects. Available at (accessed 6 Apr. 2015) http://www.wma.net/en/30publications/10policies/b3/
19. Vanharanta et al. Epigenetic expansion of VHL-HIF signal output drives multiorgan metastasis in renal cancer. Nat Med 2013; 19: 50-6.
20. Gale and McColl, Chemokines: extracellular messengers for all occasions? BioEssays 1999; 21: 17-28.
21. Highfill et al., Disruption of CXCR2-mediated MDSC tumor trafficking enhances anti-PD1 efficacy. Sci Transl Med 2014; 6: ra67.
22. Facciabene et al., Tumour hypoxia promotes tolerance and angiogenesis via CCL28 and Treg cells. Nature 2011; 475: 226-230.
23. Montane et al., Prevention of murine autoimmune diabetes by CCL22-mediated Treg recruitment to pancreatic islets. J Clin Invest 2011; 121: 3024-8.
24. Acharyya et al., CXCL1 paracrine network links cancer chemoresistance and metastasis. Cell 2012; 150: 165-78.
25. Zhao et al., TNF signaling drives myeloid-derived suppressor cell accumulation. J Clin Invest 2012; 122: 4094-4104.
26. Silva et al., Profiling essential genes in human mammary cells by multiplex RNA1 screening. Science 2008; 319: 617-20.
27. Schlabach et al., Cancer proliferation gene discovery through functional genomics. Science 2008; 319: 620-24.
28. Shen et al., CXCR4-mediated STAT3 activation is essential for CXCL12-induced invasion in bladder cancer. Tumour Biol 2013; 34: 1839-45.

We claim:

1. A method of treating cancer in a patient in need thereof, wherein said method comprises administering to said patient X4P-001 or a pharmaceutically acceptable salt thereof in combination with one or more immunostimulatory therapeutic compounds.

2. The method of claim 1, wherein the one or more immunostimulatory therapeutic compounds are selected from elotuzumab, mifamurtide, an agonist or activator of a toll-like receptor, or an activator of RORγt.

3. The method of claim 1 or 2, further comprising administering to said patient an immune checkpoint inhibitor.

4. The method of claim 3, wherein the immune checkpoint inhibitor is selected from nivolumab, pembrolizumab, ipilimumab, avelumab, durvalumab, atezolizumab, or pidilizumab.

5. A method of treating cancer in a patient in need thereof, wherein said method comprises administering to said patient X4P-001 or a pharmaceutically acceptable salt thereof in combination with one or more additional therapeutic agents selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor or a phosphatidylinositol 3 kinase (PI3K) inhibitor.

6. The method of claim 5, wherein the IDO inhibitor is selected from epacadostat, indoximod, capmanitib, GDC-0919, PF-06840003, BMS:F001287, Phy906/KD108, or an enzyme that breaks down kynurenine.

7. The method of claim 5, wherein the PARP inhibitor is selected from olaparib, rucaparib, or niraparib.

8. The method of claim 5, wherein the HDAC inhibitor is selected from vorinostat, romidepsin, panobinostat, belinostat, entinostat, or chidamide.

9. The method of claim 5, wherein the PI3K inhibitor is selected from idelalisib, alpelisib, taselisib, pictilisib, copanlisib, duvelisib, PQR309, or TGR1202.

10. The method of claim 5, wherein the CDK 4/6 inhibitor is selected from palbociclib, ribociclib, abemaciclib or trilaciclib.

11. The method of any of claims 5-10, further comprising administering to said patient an immune checkpoint inhibitor.

12. The method of claim 11, wherein the immune checkpoint inhibitor is selected from nivolumab, pembrolizumab, ipilimumab, avelumab, durvalumab, atezolizumab, or pidilizumab.

13. A method of treating cancer in a patient in need thereof, wherein said method comprises administering to said patient X4P-001 or a pharmaceutically acceptable salt thereof in combination with one or more additional therapeutic agents selected from a platinum-based therapeutic, a taxane, a nucleoside inhibitor, or a therapeutic agent that interferes with normal DNA synthesis, protein synthesis, cell replication, or will otherwise inhibit rapidly proliferating cells.

14. The method of claim 13, wherein the platinum-based therapeutic is selected from cisplatin, carboplatin, oxaliplatin, nedaplatin, picoplatin, or satraplatin.

15. The method of claim 13, wherein the taxane is selected from paclitaxel, docetaxel, albumin-bound paclitaxel, cabazitaxel, or SID530.

16. The method of claim 13, wherein the therapeutic agent that interferes with the replication of rapidly proliferating cells is selected from trabectedin, mechlorethamine, vincristine, temozolomide, cytarabine, lomustine, azacitidine, omacetaxine mepesuccinate, asparaginase Envinia chrysanthemi, eribulin mesylate, capacetrine, bendamustine, ixabepilone, nelarabine, clorafabine, trifluridine, or tipiracil.

17. The method of any of claims 13-16, further comprising administering to said patient an immune checkpoint inhibitor.

18. The method of claim 17, wherein the immune checkpoint inhibitor is selected from nivolumab, pembrolizumab, ipilimumab, avelumab, durvalumab, atezolizumab, or pidilizumab.

19. The method of any one of claims 1-18, further comprising the step of obtaining a biological sample from the patient and measuring the amount of a disease-related biomarker.

20. The method of claim 19, wherein the biological sample is a blood sample.

21. The method of claim 20, wherein the disease-related biomarker is selected from circulating CD8+ T cells or the ratio of CD8+ T cells:Treg cells.

22. The method of any of claims 1-21, wherein the cancer is selected from hepatocellular carcinoma, ovarian cancer, ovarian epithelial cancer, fallopian tube cancer, papillary serous cystadenocarcinoma or uterine papillary serous carcinoma (UPSC); prostate cancer, testicular cancer, gallbladder cancer, hepatocholangiocarcinoma, soft tissue and bone synovial sarcoma; rhabdomyosarcoma, osteosarcoma, chondrosarcoma, Ewing sarcoma, anaplastic thyroid cancer, adrenocortical adenoma, pancreatic cancer, pancreatic ductal carcinoma or pancreatic adenocarcinoma; gastrointestinal/stomach (GIST) cancer, lymphoma, squamous cell carcinoma of the head and neck (SCCHN), salivary gland cancer, glioma or brain cancer; neurofibromatosis-1 associated malignant peripheral nerve sheath tumors (MPNST), Waldenstrom's macroglobulinemia, or medulloblastoma.

23. The method of any of claims 1-21, wherein the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.
